(19) United States
(12) Patent Application Publication
(10) Pub. No.: US 2022/0306728 A1
(43) Pub. Date: Sep. 29, 2022

(54) COMPOSITIONS AND METHODS FOR TREATMENT OF INFLUENZA A INFECTION

(71) Applicant: VIR BIOTECHNOLOGY, INC., San Francisco, CA (US)

(72) Inventors: Phillip S. PANG, San Francisco, CA (US); Lynn E. CONNOLLY, San Francisco, CA (US); Erik MOGALIAN, San Francisco, CA (US)

(21) Appl. No.: 17/638,630

(22) PCT Filed: Aug. 28, 2020
(86) PCT No.: PCT/US2020/048635

§ 371 Date: Feb. 25, 2022

Related U.S. Application Data
(60) Provisional application No. 63/040,966, filed on Jun. 18, 2020.
(60) Provisional application No. 62/993,519, filed on Mar. 23, 2020.
(60) Provisional application No. 62/893,747, filed on Aug. 29, 2019.

(51) Int. Cl. C07K 16/10; A61K 47/26; A61K 47/22; A61P 31/16

(52) U.S. Cl. CPC C07K 16/1018; A61K 47/26; A61K 47/22; A61P 31/16; A61K 39/00

(57) ABSTRACT

The present disclosure provides antibodies, antibody compositions, and methods for use in prophylaxis and treatment of influenza A infection. In certain embodiments, a single administration of a presently disclosed antibody or antibody composition is useful to protect against and/or treat an influenza A infection for a full flu season.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930485_413WO_SEQUENCE_LISTING.txt. The text file is 12.2 KB, was created on Aug. 26, 2020, and is being submitted electronically via EFS-Web.

The present disclosure relates to antibody compositions and methods for prophylaxis and treatment of influenza A infection.

### BACKGROUND

Influenza is an infectious disease, which spreads around the world in yearly outbreaks resulting per year in about three to five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although symptoms usually begin about two days after exposure to the virus. Complications of influenza may include pneumonia, sinus infections, and worsening of existing health problems such as asthma or heart failure, sepsis or exacerbation of chronic underlying diseases.

Influenza is caused by influenza virus, an antigenically and genetically diverse group of viruses of the family Orthomyxoviridae that contains a negative-sense, single-stranded, segmented RNA genome. Of the four types of influenza virus (A, B, C and D), three types (A, B and C) affect humans. Influenza type A viruses are the most virulent human pathogens and cause the severest disease. Influenza A viruses can be categorized based on the different subtypes of major surface proteins present: Hemagglutinin (HA) and Neuraminidase (NA). There are at least 18 influenza A subtypes defined by their hemagglutinin (“HA”) proteins. HAs can be classified into two groups. Group 1 contains H1, H2, H5, H6, H8, H9, H11, H12, H13, H16 and H17 subtypes, and group 2 includes H3, H4, H7, H10, H14 and H15 subtypes. While all subtypes are present in birds, mostly H1, H2 and H3 subtypes cause disease in humans. H5, H7 and H9 subtypes are causing sporadic severe infections in humans and may generate a new pandemic. Influenza A viruses continuously evolve generating new variants, a phenomenon called antigenic drift. As a consequence, antibodies produced in response to past viruses are poorly- or non-protective against new drifted viruses. A consequence is that a new vaccine has to be produced every year against H1 and H3 viruses that are predicted to emerge, a process that is very costly as well as not always efficient. The same applies to the production of a H5 influenza vaccine.

HA is a major surface protein of influenza A virus, which is the main target of neutralizing antibodies that are induced by infection or vaccination. HA is responsible for binding the virus to cells with sialic acid on the membranes, such as cells in the upper respiratory tract or erythrocytes. In addition, HA mediates the fusion of the viral envelope with the endosome membrane, after the pH has been reduced. HA is a homotrimeric integral membrane glycoprotein. The HA trimer is composed of three identical monomers, each made of an intact HA0 single polypeptide chain with HA1 and HA2 regions linked by 2 disulfide bridges. Each HA2 region adopts alpha helical coiled coil structure and primarily forms the “stem” or “stalk” region of HA, while the HA1 region is a small globular domain containing a mix of α/β structures (“head” region of HA). The globular HA head region mediates binding to the sialic acid receptor, while the HA stem mediates the subsequent fusion between the viral and cellular membranes that is triggered in endosomes by the low pH. While the immunodominant HA globular head domain has high plasticity with distinct antigenic sites undergoing constant antigenic drift, the HA stem region is relatively conserved among subtypes. Current influenza vaccines mostly induce an immune response against the immunodominant and variable HA head region, which evolves faster than the stem region of HA (Kirkpatrick E, Qiu X, Wilson P C, Bahl J, Krammer F. The influenza virus hemagglutinin head evolves faster than the stalk domain. Sci Rep. 2018 Jul 11; 8(1):10432). Therefore, a particular influenza vaccine usually confers protection for no more than a few years and annual re-development of influenza vaccines is required.

To overcome these problems, recently a new class of influenza-neutralizing antibodies that target conserved sites in the HA stem were developed as influenza virus therapeutics. These antibodies targeting the stem region of HA are usually broader neutralizing compared to antibodies targeting the head region of HA. An overview over broadly neutralizing influenza A antibodies is provided in Corti D. and Lanzavecchia A., Broadly neutralizing antiviral antibodies. Annu. Rev. Immunol. 2013; 31:705-742. Okuno et al. immunized mice with influenza virus A/Okuda/57 (H2N2) and isolated a monoclonal antibody (C179) that binds to a conserved conformational epitope in HA2 and neutralizes the Group 1 H2, H1 and H5 subtype influenza A viruses in vitro and in vivo in animal models (Okuno et al.,1993; Smirnov et al., 1999; Smirnov et al., 2000). Further examples of HA-stem region targeting antibodies include CR6261 (Throsby M, van den Brink E, Jongeneelen M, Poon L L M, Alard P, Cornelissen L, et al. (2008) Heterosubtypic Neutralizing Monoclonal Antibodies Cross-Protective against H5N1 and H1N1 Recovered from Human IgM⁺ Memory B Cells. PLoS ONE 3(12): e3942. https://doi.org/10.1371/journal.pone.0003942; Friesen R H E, Koudstaal W, Koldijk M H, Weverling G J, Brakenhoff J P J, Lenting P J, et al. (2010) New Class of Monoclonal Antibodies against Severe Influenza: Prophylactic and Therapeutic Efficacy in Ferrets. PLoS ONE 5(2): e9106. https://doi.org/10.1371/journal.pone.0009106), F10 (Sui J, Hwang W C, Perez S, Wei G, Aird D, Chen L M, Santelli E, Stec B, Cadwell G, Ali M, Wan H, Murakami A, Yammanuru A, Han T, Cox N J, Bankston L A, Donis R O, Liddington R C, Marasco W A (March 2009). “Structural and functional bases for broad-spectrum neutralization of avian and human influenza A viruses”. Nature Structural & Molecular Biology. 16 (3): 265-73. doi:10.1038/nsmb.1566), CR8020 (Ekiert D C, Friesen R H E, Bhabha G, Kwaks T, Jongeneelen M, et al. 2011. A highly conserved neutralizing epitope on group 2 influenza A viruses. Science 333(6044):843-50), FI6 (Corti D, Voss J, Gamblin S J, Codoni G, Macagno A, et al. 2011. A neutralizing antibody selected from plasma cells that binds to group 1 and group 2 influenza A hemagglutinins. Science 333(6044):850-56), and CR9114 (Dreyfus C, Laursen N S, Kwaks T, Zuijdgeest D, Khayat R, et al. 2012. Highly conserved protective epitopes on influenza B viruses. Science 337(6100):1343-48).

However, antibodies capable of reacting with the HA stem region of both group 1 and 2 subtypes are extremely rare and usually do not show complete coverage of all subtypes. Recently, antibody MEDI8852 was described, which neutralizes group 1 and 2 influenza A viruses, being able to neutralize a panel of representative viruses spanning >80 years of antigenic evolution (Kallewaard N L, Corti D, Collins P J, et al. Structure and Function Analysis of an Antibody Recognizing All Influenza A Subtypes. Cell. 2016; 166(3):596-608; Paules, C. I. et al. The Hemagglutinin A Stem Antibody MEDI8852 Prevents and Controls Disease and Limits Transmission of Pandemic Influenza Viruses. J Infect Dis 216, 356-365, https://doi.org/10.1093/infdis/jix292 (2017)). MEDI8852 was shown to bind to a highly conserved epitope that is markedly different from other structurally characterized stem-reactive neutralizing antibodies (Kallewaard N L, Corti D, Collins P J, et al. Structure and Function Analysis of an Antibody Recognizing All Influenza A Subtypes. Cell. 2016; 166(3):596-608).

Despite continued efforts, a need remains for antibody compositions and methods that may be used for the prophylaxis and/or treatment of influenza A.

### DETAILED DESCRIPTION

The present disclosure provides pharmaceutical compositions including antibodies that neutralize influenza A and methods of using those compositions. In certain embodiments, the pharmaceutical compositions include an antibody that neutralizes influenza A and maintains systemic exposure in a subject for a period selected from at least 10 weeks, at least 15 weeks, and at least 20 weeks after a single administration. In specific embodiments, the antibody and the pharmaceutical composition are well-tolerated by the subject when administered in amounts that are prophylactically effective. In other embodiments, the antibody and the pharmaceutical composition are well-tolerated by the subject when administered in amounts that are therapeutically effective. In some embodiments, the methods described herein include administering an antibody composition according to the present description to a subject at risk of infection by influenza A. In still other embodiments, the methods described herein include administering an antibody composition according to the present description to a subject infected by influenza A.

Though antibodies that neutralize influenza A, pharmaceutical compositions including those antibodies, and methods for using such compositions are described in detail below, it is to be understood that this disclosure is not limited to the particular methodologies, protocols and reagents described herein as these may vary. It is also to be understood that the terminology used herein is not intended to limit the scope of the present disclosure. Unless defined otherwise, all technical and scientific terms used herein have the same meanings as commonly understood by one of ordinary skill in the art.

In the following, aspects of the present disclosure are described. Certain embodiments are provided, however, it should be understood that embodiments of the disclosure may be combined in any manner and in any number to create additional embodiments. The variously described examples and embodiments should not be construed to limit the present disclosure to only explicitly described embodiments. This description should be understood to support and encompass embodiments which combine explicitly described embodiments with any disclosed subject-matter. Furthermore, any permutations and combinations of all described subject-matter in this application should be considered disclosed by the description of the present application unless the context indicates otherwise.

Throughout this disclosure, unless the context requires otherwise, the term “comprise”, and variations such as “comprises” and “comprising” (or “having,” “has,” “including,” “includes,” or the like, will be understood to imply the inclusion of a stated member, ratio, integer (including, where appropriate, a fraction thereof; e.g., one tenth and one hundredth of an integer), concentration, or step but not the exclusion of any other non-stated member, integer, concentration, or step.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “consist of” is a particular embodiment of the term “comprise”, wherein any other non-stated member, integer or step is excluded. In the context of the present disclosure, the term “comprise” encompasses the term “consist of”. The term “comprising” thus encompasses “including” as well as “consisting” e.g., a composition “comprising” X may consist exclusively of X or may include something additional e.g., X+Y.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The terms “a” and “an” and “the” and similar reference used in the context of describing the disclosure (including in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the subject-matter disclosed herein.

The word “substantially” does not exclude “completely” e.g., a composition which is “substantially free” from Y may be completely free from Y. In certain embodiments, “substantially” refers to a given amount, effect, or activity of a composition, method, or use of the present disclosure as compared to that of a reference composition, method, or use, and describes a reduction in the amount, effect, or activity of no more than 50%, such as no more than 40%, 30%, 25%, 20%, 15%, 10%, 5%, or 1%, or less, of the amount, effect, or activity of the reference composition, method, or use.

The term “about” in relation to a numerical value x means x±10%, for example, x±5%, or x±7%, or x±10%, or x±12%, or x±15%, or x±20%. For example, in certain embodiments, “about” means±20% of the indicated range, value, or structure.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “therapeutically effective” refers to the nature or amount of a pharmaceutical composition or antibody as described herein that is sufficient to provide a benefit to the subject. In the context of the present disclosure, the benefit provided to the subject is treatment of influenza A infection. As used herein, reference to “treatment” includes prevention, prophylaxis, attenuation, amelioration and therapy. Benefits of treatment include improved clinical outcome; lessening or alleviation of symptoms associated with infection; decreased occurrence of symptoms; improved quality of life; longer disease-free status; prevention of infection; diminishment of extent and/or duration of infection; stabilization of disease state; delay of disease progression; remission; survival; prolonged survival; or any combination thereof. Therefore, in certain embodiments, “therapeutically effective” encompasses both treatment of subjects infected with influenza A, as well as prevention or prophylaxis of infection with influenza A in a subject. Where the intended treatment is prophylaxis, the terms “therapeutically effective” and “prophylactically effective” may be used interchagably. The terms “subject” or “patient” are used interchangeably herein to mean human subjects that are susceptible to infection by influenza A or have already been infected by influenza A.

In certain embodiments, an influenza A virus comprises a H1N1 virus, an H3N2 virus, or both.

Doses are often expressed in relation to bodyweight (i.e., of a subject). Thus, a dose which is expressed as [g, mg, or other unit]/kg (or g, mg etc.) can refer to [g, mg, or other unit] “per kg (or g, mg etc.) bodyweight”, even if the term “bodyweight” is not explicitly mentioned.

The term “specifically binding” and similar reference does not encompass non-specific sticking.

As used herein, the term “antibody” encompasses various forms of antibodies including, without being limited to, whole antibodies (comprising at least two heavy (H) chains and two light (L) chains inter-connected by disulfide bonds (though it will be understood that heavy chain antibodies, which lack light chains, are still encompassed by the term “antibody”)), antibody fragments that have or retain the ability to bind to the antigen target molecule recognized by the intact antibody, such as, for example, a scFv, Fab, or F(ab′)2 fragment, human antibodies, chimeric antibodies, humanized antibodies, recombinant antibodies and genetically and otherwise engineered or modified antibodies (e.g., variant or mutant antibodies, intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv, and other antibody formats known in the art) as long as the characteristic properties according to the disclosure are retained. Thus, the term “antibody” herein is used in the broadest sense and includes polyclonal and monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. In some embodiments, the antibody is a human antibody. In some embodiments, the antibody is a monoclonal antibody. For example, the antibody is a human monoclonal antibody. Unless otherwise stated, the term “antibody” should be understood to encompass functional antibody fragments thereof (i.e., comprising or consisting of an antigen-binding fragment of the antibody that retains the ability to bind antigen). The term also encompasses intact or full-length antibodies, including antibodies of any class or sub-class thereof, including IgG and sub-classes thereof (IgG1, IgG2, IgG2, IgG4), IgM, IgE, IgA, and IgD.

Accordingly, antibodies of the disclosure can be of any isotype (e.g., IgA, IgG, IgM, also referred to as α, γ and μ heavy chain, respectively). For example, in certain embodiments, antibody is of the IgG type. Within the IgG isotype, antibodies may be IgG1, IgG2, IgG3 or IgG4 subclass, for example IgG1. In some embodiments, an antibody comprises an amino acid sequence from two different isotypes (e.g., exchange of constant domain amino acid sequence), such as, for example, an antibody comprising a constant region that comprises amino acid sequence from an IgA antibody and amino acid sequence from an IgG antibody. Antibodies of the disclosure may comprise a κ or a λ light chain. In some embodiments, the antibody is of IgG1 type and comprises a κ light chain.

Human antibodies are known (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5.

As used herein, the term “variable region” (e.g., variable region of a light chain (V_{L}), variable region of a heavy chain (V_{H})) refers to the variable region of an antibody light chain or an antibody heavy chain, which is involved directly in binding the antibody to the antigen. In other words, the terms “V_{L}” or “VL” and “V_{H}” or “VH” refer to the variable binding region from an antibody light chain and an antibody heavy chain, respectively.

Antibodies included in the pharmaceutical compositions and methods described herein typically comprise (at least) three complementarity determining regions (CDRs) on a heavy chain (or heavy chain variable region) and (at least) three CDRs on a light chain (or light chain variable region). Complementarity determining regions (CDRs) are the hypervariable regions (“HVRs”); CDRs are synonymous with HVRs) present in heavy chain variable domains and light chain variable domains. Typically, the CDRs of a heavy chain and a cognate light chain of an antibody together form the antigen-binding site (and in general, together confer the antigen specificity and/or binding affinity of the antibody). Usually, the three CDRs (CDR1, CDR2, and CDR3) are separated by framework sequence in the variable domain. In general, there are six CDRs for each antigen-binding site (heavy chain: CDRH1, CDRH2, and CDRH3; light chain: CDRL1, CDRL2, and CDRL3). For example, a single antibody molecule comprising two antigen-binding site contains twelve CDRs. The CDRs on the heavy and/or light chain may be separated in primary amino acid sequence by framework regions, whereby a framework region (FR) is a region in the variable domain which is less variable (i.e., from one antibody to another (e.g., from one antibody to another encoded by a same allele or alleles)) than the CDR. For example, a chain (or each chain, respectively) may be composed of four framework regions, separated by three CDRs. In certain embodiments, an antibody VH comprises four FRs and three CDRs arranged as follows: FR1-CDRH1-FR2-CDRH2-FR3-CDRH3-FR4; and an antibody VL comprises four FRs and three CDRs as follows: FR1-CDRL1-FR2-CDRL2-FR3-CDRL3-FR4. In general, the VH and the VL together form the antigen-binding site through their respective CDRs, though it will be understood that in some cases, a binding site can be formed by or comprise one, two, three, four, or five of the CDRs.

The sequences of the heavy chains and light chains of exemplary antibodies of the disclosure, comprising three different CDRs on the heavy chain and three different CDRs on the light chain were determined. The position of the CDR amino acids are defined according to the IMGT numbering system (IMGT: http://www.imgt.org/; cf. Lefranc, M.-P. et al. (2009) Nucleic Acids Res. 37, D1006-D1012).

In some embodiments, the antibody will be present in a pharmaceutical composition that is substantially free of other polypeptides e.g., where less than 90% (by weight), or less than 60%, or less than 50% of the pharmaceutical composition is made up of other polypeptides.

Antibodies according to the present disclosure may be immunogenic in human and/or in non-human (or heterologous) hosts, e.g., in mice. For example, the antibodies may have an idiotope that is immunogenic in non-human hosts, but not in a human host. Antibodies of the disclosure for human use include those that cannot be easily isolated from hosts such as mice, goats, rabbits, rats, non-primate mammals, etc. and cannot generally be obtained by humanization or from xeno-mice. In certain embodiments, an antibody according to the present disclosure is non-immunogenic or is substantially non-immunogenic in a human.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are referenced in this disclosure. Each of the documents referenced herein (including all patents, patent applications, scientific publications, manufacturer's specifications, instructions, etc.), whether supra or infra, are hereby incorporated by reference in their entirety. Nothing herein is to be construed as an admission that the disclosure is not entitled to antedate such disclosure by virtue of prior disclosure.

It is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Antibodies

Antibodies for use in the pharmaceutical compositions and methods described herein neutralize influenza A virus. In addition, the antibodies of the disclosure show an in vivo half-life that maintains systemic exposure over an extended period of time when administered to a subject at well-tolerated doses. In certain embodiments, the antibodies described herein neutralize influenza A and maintain systemic exposure in a subject for a period selected from at least 10 weeks, at least 15 weeks, and at least 20 weeks after a single administration. In specific embodiments, when compared to a comparator or reference antibody, antibodies included in the pharmaceutical compositions and methods of the present disclosure show increased potency despite similar plasma concentrations of the antibody as compared to the comparator or reference antibody.

Antibodies for use in the pharmaceutical compositions and methods described herein bind to hemagglutinin of an influenza A virus and can thereby neutralize infection of influenza A virus. In particular embodiments, the antibody according to the present disclosure binds to the same epitope of the influenza A virus hemagglutinin (IAV HA) stem region as MEDI8852 (Kallewaard N L, Corti D, Collins P J, et al. Structure and Function Analysis of an Antibody Recognizing All Influenza A Subtypes. Cell. 2016; 166(3):596-608), thereby providing broad protection against various influenza A serotypes of all influenza A subtypes.

In addition, antibodies suitable for use in the pharmaceutical compositions and methods of the present disclosure include two mutations in the constant region of the heavy chain (in the CH3 region): M428L and N434S. In this context, the amino acid positions have been numbered according to the art-recognized EU numbering system. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E. A., National Institutes of Health (U.S.) Office of the Director, “Sequences of Proteins of Immunological Interest”, 5^{th} edition, Bethesda, Md. U.S. Dept. of Health and Human Services, Public Health Service, National Institutes of Health, 1991, hereby entirely incorporated by reference).

To study and quantitate virus infectivity (or “neutralization”) in the laboratory the person skilled in the art knows various standard “neutralization assays”. For a neutralization assay animal viruses are typically propagated in cells and/or cell lines. For example, in a neutralization assay cultured cells may be incubated with a fixed amount of influenza A virus (IAV) in the presence (or absence) of the antibody to be tested. As a readout for example flow cytometry may be used. Alternatively, also other readouts are conceivable.

In some embodiments, the antibody of the disclosure is a human antibody. In some embodiments, the antibody of the disclosure is a monoclonal antibody. For example, the antibody of the disclosure is a human monoclonal antibody.

Antibodies of the disclosure can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the disclosure may have a κ or a λ light chain. In some embodiments, the antibody has a kappa (κ) light chain. In some embodiments, the antibody is of IgG1 type and has a κ light chain.

In some embodiments, the pharmaceutical compositions and methods described herein include an antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S (according to EU numbering) in the constant region of the heavy chain.

In some embodiments, the pharmaceutical compositions and methods described herein include an (isolated) antibody comprising a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 70% identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are maintained.

Sequence identity is usually calculated with regard to the full length of a reference sequence (i.e. a sequence as recited or referenced herein). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the reference sequence. By way of illustration, a variant sequences that is least 70% identical to the reference sequence has no more than 30 alterations, i.e. any combination of deletions, insertions or substitutions, per 100 amino acids of the reference sequence.

In general, while it is possible to have non-conservative amino acid substitutions, substitutions can comprise conservative amino acid substitutions, in which the substituted amino acid has similar structural (e.g., side chain) or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

As a further example, conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency. In particular embodiments a functional variant performs at least one function of the parent polypeptide with an efficiency selected from at least 55%, 60%, 70%, 75%, 80%, 85%, 90%, 95%, 96%, 97%, 98%, 99%, 99.9%, or 100% efficiency. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant).

As used herein, a “functional portion” or “functional fragment” refers to a polypeptide or polynucleotide that comprises only a domain, portion or fragment of a parent or reference compound, and the polypeptide or encoded polypeptide of the functional portion or functional fragment retains at least 50% activity associated with the domain, portion or fragment of the parent or reference compound. In particular embodiments a functional portion or functional fragment retains at least 55%, 60%, 70%, 75%, 80%, 85%, 90%, 95%, 96%, 97%, 98%, 99%, 99.9%, or 100% activity associated with the domain, portion or fragment of the parent polypeptide. In some such embodiments, a functional portion or functional fragment additionally provides a biological benefit (e.g., effector function). A functional portion or functional fragment of a polypeptide or encoded polypeptide of this disclosure has “similar binding” or “similar activity” when the functional portion or fragment displays no more than a 50% reduction in performance in a selected assay as compared to the parent or reference polypeptide. In specific embodiments, similar binding and similar activity refer percent reduction selected from no more than 20%, no more than 10%, and no more than a log difference as compared to the parent or reference with regard to affinity.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

In certain embodiments, an antibody of a pharmaceutical composition can be “isolated”, e.g., in that it is removed from, separated from, or not otherwise associated with the in vivo environment of a subject.

The term “gene” means the segment of DNA or RNA involved in producing a polypeptide chain;

In certain contexts, it includes regions preceding and following the coding region (e.g., 5′ untranslated region (UTR) and 3′ UTR) as well as intervening sequences (introns) between individual coding segments (exons).

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

The term “recombinant”, as used herein (e.g. a recombinant antibody, a recombinant protein, a recombinant nucleic acid, or the like), refers to any molecule (antibody, protein, nucleic acid, or the like) which is prepared, expressed, created or isolated by recombinant means, and which is not naturally occurring. “Recombinant” can be used synonymously with “engineered” or “non-natural” and can refer to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof.

A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

In some embodiments, an antibody suitable for use in the pharmaceutical compositions and methods provided herein comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to the amino acid sequence as set forth in SEQ ID NO: 7 and a light chain variable region comprising the amino acid sequence having 75% or more identity to the amino acid sequence as set forth in SEQ ID NO: 8, wherein the CDR sequences as defined above are maintained. In some embodiments, an antibody suitable for use in the pharmaceutical compositions and methods provided herein comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to the amino acid sequence as set forth in SEQ ID NO: 7 and a light chain variable region comprising the amino acid sequence having 80% or more identity to the amino acid sequence as set forth in SEQ ID NO: 8, wherein the CDR sequences as defined above are maintained. In some embodiments, an antibody suitable for use in the pharmaceutical compositions and methods provided herein comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to the amino acid sequence as set forth in SEQ ID NO: 7 and a light chain variable region comprising the amino acid sequence having 85% or more identity to the amino acid sequence as set forth in SEQ ID NO: 8, wherein the CDR sequences as defined above are maintained. In some embodiments, the antibody of the disclosure comprises a heavy chain variable region comprising an amino acid sequence having 90% or more (i.e. 91%, 92%, 93%, 94%, 95%, 96%, 97%, 98%, 99% or more) identity to the amino acid sequence as set forth in SEQ ID NO: 7 and a light chain variable region comprising the amino acid sequence having 90% or more identity to the amino acid sequence as set forth in SEQ ID NO: 8, wherein the CDR sequences as defined above are maintained. In some embodiments, an antibody suitable for use in the pharmaceutical compositions and methods provided herein comprises a heavy chain variable region comprising an amino acid sequence having 95% or more (i.e. 95% 96%, 97%, 98%, 99% or more) identity to the amino acid sequence as set forth in SEQ ID NO: 7 and a light chain variable region comprising the amino acid sequence having at least 95% identity to the amino acid sequence as set forth in SEQ ID NO: 8, wherein the CDR sequences as defined above are maintained.

In some embodiments, an antibody suitable for use in the pharmaceutical compositions and methods provided herein comprises a heavy chain variable region comprising or consisting of the amino acid sequence as set forth in SEQ ID NO: 7 and a light chain variable region comprising or consisting of the amino acid sequence as set forth in SEQ ID NO: 8, wherein the CDR sequences as defined above are maintained.

In general, it is possible that the antibodies described herein may comprise one or more further mutations (in addition to M428L and N434S) in the Fc region (e.g., in the CH2 or CH3 region). However, in some embodiments, an antibody suitable for use in the pharmaceutical compositions and methods provided herein does not comprise any further mutation in addition to M428L and N434S in its CH3 region (in comparison to the respective wild-type CH3 region). In some embodiments, an antibody suitable for use in the pharmaceutical compositions and methods provided herein does not comprise any further mutation in addition to M428L and N434S in its Fc region (in comparison to the respective wild-type Fc region). As used herein, the term “wild-type” refers to the reference sequence, for example as occurring in nature. As a specific example, the term “wild-type” may refer to the sequence with the highest prevalence occurring in nature.

In some embodiments, an antibody suitable for use in the pharmaceutical compositions and methods provided herein comprises a heavy chain comprising or consisting of the amino acid sequence as set forth in SEQ ID NO: 9 and a light chain comprising or consisting of the amino acid sequence as set forth in SEQ ID NO: 10. For example, the antibody of the disclosure may include a heavy chain consisting of the amino acid sequence as set forth in SEQ ID NO: 9 and a light chain consisting of the amino acid sequence as set forth in SEQ ID NO: 10.

Variant antibodies are also included within the scope of the disclosure. Thus, variants of the sequences recited in the application are also included within the scope of the disclosure. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Nucleic Acids

In another aspect, the disclosure also provides a nucleic acid molecule comprising a polynucleotide encoding an antibody as described herein, or a portion thereof. Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of the antibody of the disclosure. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

Due to the redundancy of the genetic code, the present disclosure also comprises sequence variants of nucleic acid sequences, which encode the same amino acid sequences. Accordingly, a nucleic acid molecule encoding an amino acid sequence includes all nucleotide sequences that encode the same amino acid sequence. A polynucleotide encoding the antibody (or the complete nucleic acid molecule) may be optimized for expression of the antibody in a host cell. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light) chain of an antibody. For example, a nucleic acid molecule may comprise a heterologous promotor, a heterologous enhancer, a heterologous UTR (e.g., for optimal translation/expression), a heterologous Poly-A-tail, and the like.

Some versions of the nucleotide sequences may also include intron(s) to the extent that the intron(s) may be removed through co- or post-transcriptional mechanisms. Different nucleotide sequences may encode the same amino acid sequence as the result of the redundancy or degeneracy of the genetic code, or by splicing, or both.

A nucleic acid molecule is a molecule comprising nucleic acid components. The term nucleic acid molecule usually refers to DNA (including cDNA, genomic DNA, and synthetic DNA) or RNA molecules, either of which may be single or double-stranded. If single-stranded, the nucleic acid molecule may be the coding strand or non-coding (anti-sense strand). Polynucleotides (including oligonucleotides), and fragments thereof may be generated, for example, by polymerase chain reaction (PCR) or by in vitro translation, or generated by any of ligation, scission, endonuclease action, or exonuclease action. It may be used synonymously with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

For example, a nucleic acid molecule can comprise nucleotides comprising natural subunits (e.g., purine or pyrimidine bases) and/or non-natural subunits (e.g., morpholine ring). Purine bases include adenine, guanine, hypoxanthine, and xanthine, and pyrimidine bases include uracil, thymine, and cytosine. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, or the like.

A nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody of the disclosure can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, J Cat: a novel tool to adapt codon usage of a target gene to its potential expression host. Nucleic Acids Res. 2005 Jul. 1; 33(Web Server issue):W526-31; or, for example, Genscript's OptimumGene™ algorithm (as described in US 2011/0081708 A1); or the GeneArt Gene Synthesis Tool (Thermo Fisher Scientific). Codon-optimized sequences include sequences that are partially codon-optimized (i.e., at least one codon is optimized for expression in the host cell) and those that are fully codon-optimized.

The present disclosure also provides a combination of a first and a second nucleic acid molecule, wherein the first nucleic acid molecule comprises a polynucleotide encoding the heavy chain of the antibody of the present disclosure; and the second nucleic acid molecule comprises a polynucleotide encoding the corresponding light chain of the same antibody. The above description regarding the (general) features of the nucleic acid molecule of the disclosure applies accordingly to the first and second nucleic acid molecule of the combination. For example, one or both of the polynucleotides encoding the heavy and/or light chain(s) of the antibody may be codon-optimized.

As used herein, a nucleic acid sequence or an amino acid sequence “derived from” a designated nucleic acid, peptide, polypeptide or protein refers to the origin of the nucleic acid, peptide, polypeptide or protein. A nucleic acid sequence or amino acid sequence which is derived from a particular sequence may have an amino acid sequence that is essentially identical to that sequence or a portion thereof, from which it is derived, whereby “essentially identical” includes sequence variants as defined above. A nucleic acid sequence or amino acid sequence which is derived from a particular peptide or protein, may be derived from the corresponding domain in the particular peptide or protein. In this context, “corresponding” refers to possession of a same functionality or characteristic of interest. “Corresponding” parts of peptides, proteins and nucleic acids are thus easily identifiable to one of ordinary skill in the art. Likewise, a sequence “derived from” another (e.g., “source”) sequence can be identified by one of ordinary skill in the art as having its origin in the source sequence.

Vector

Further included within the scope of the disclosure are vectors, for example, expression vectors, comprising a nucleic acid molecule according to the present disclosure. Usually, a vector comprises a nucleic acid molecule as described above.

The present disclosure also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present disclosure is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present disclosure. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present disclosure may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

Cells

In a further aspect, the present disclosure also provides cells expressing an antibody according to the present disclosure; and/or comprising a vector according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980), CHO-KSV, ExpiCHO), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibodies in bacteria, see, e.g., U.S. Pat. Nos. 5,648,237; 5,789,199; and 5,840,523.

Insect cells useful for expressing an antibody of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

Any protein expression system compatible with the disclosure may be used to produce the disclosed antibodies. Suitable expression systems include transgenic animals described in Gene Expression Systems, Academic Press, eds. Fernandez et al., 1999.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine etc. In certain embodiments, the introduction is non-viral.

Moreover, cells of the present disclosure may be transfected stably or transiently with the vector according to the present description, e.g. for expressing an antibody thereof, according to the present description. In such embodiments, the cells are stably transfected with the vector as described herein encoding a binding protein. Alternatively, cells may be transiently transfected with a vector according to the present disclosure encoding an antibody according to the present description. In any of the presently disclosed embodiments, a polynucleotide may be heterologous to the host cell.

In a related aspect, the present disclosure provides methods for producing an antibody, wherein the methods comprise culturing a host cell of the present disclosure under conditions and for a time sufficient to produce the antibody.

Accordingly, the present disclosure also provides recombinant host cells that heterologously express an antibody of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody in nature. Moreover, the host cell may impart a post-translational modification (PTM; e.g., glycosylation or fucosylation) on the binding protein that is not present in a native state of the binding protein (or in a native state of a parent binding protein from which the subject binding protein was engineered or derived). Such a PTM may result in a functional difference (e.g., reduced immunogenicity). Accordingly, a binding protein of the present disclosure that is produced by a host cell as disclosed herein may include one or more post-translational modification that is distinct from the binding protein or parent binding protein in its native state (e.g., a human antibody produced by a CHO cell can comprise a post-translational modification that is distinct from the antibody when isolated from the human and/or produced by the native human B cell or plasma cell).

Production of Antibodies

Antibodies suitable for use in the pharmaceutical compositions and methods described herein can be made by any method known in the art. For example, the general methodology for making monoclonal antibodies using hybridoma technology is well known (Kohler, G. and Milstein, C. 1975; Kozbar et al. 1983). In some embodiments, the alternative EBV immortalization method described in WO2004/076677 is used.

In some embodiments, the method as described in WO 2004/076677, which is incorporated herein by reference, is used. In this method B cells producing the antibody of the disclosure are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present disclosure. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present disclosure. Eukaryotic, e.g., mammalian, host cell expression systems may be used for production of antibody molecules, such as complete antibody molecules. Suitable mammalian host cells include those disclosed herein, such as, but not limited to, CHO, HEK293T, PER.C6, NS0, myeloma or hybridoma cells.

The present disclosure also provides a process for the production of an antibody molecule that may be included in the pharmaceutical compositions and methods described herein. In one embodiment, the process comprises culturing a (heterologous) host cell comprising a vector encoding a nucleic acid of the present disclosure under conditions suitable for expression of protein from DNA encoding the antibody molecule of the present disclosure, and isolating the antibody molecule.

For production of the antibody comprising heavy and light chains, a cell line may be transfected with two vectors, a first vector encoding a light chain polypeptide and a second vector encoding a heavy chain polypeptide. Alternatively, a single vector may be used, the vector including sequences encoding a light chain polypeptide and a heavy chain polypeptide.

Antibodies as described herein may be produced by (i) expressing a nucleic acid sequence according to the disclosure in a host cell, e.g. by use of a vector according to the present disclosure, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells described herein can be used in various ways, e.g., as a source of monoclonal antibodies, as a source of nucleic acid (DNA or mRNA) encoding a monoclonal antibody of interest, for research, etc.

The disclosure also provides a composition comprising immortalized B memory cells or transfected host cells that produce antibodies according to the present disclosure.

The immortalized B cell clone or the cultured plasma cells of the disclosure may also be used as a source of nucleic acid for the cloning of antibody genes for subsequent recombinant expression. Expression from recombinant sources may be more common for pharmaceutical purposes than expression from B cells or hybridomas, e.g., for reasons of stability, reproducibility, culture ease, etc.

Thus the disclosure also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the disclosure also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the disclosure also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the disclosure. Thus the procedures for first preparing the nucleic acid(s) and then using it to transfect a host cell can be performed at different times by different people in different places (e.g., in different countries).

These recombinant cells of the disclosure can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell can include any host cell disclosed herein, including, for example, a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In some embodiments, the transfected host cell is a mammalian cell, such as a human cell. In some embodiments, expression hosts can glycosylate the antibody of the disclosure, particularly with carbohydrate structures that are not themselves immunogenic in humans. In some embodiments the transfected host cell may be able to grow in serum-free media.

In further embodiments the transfected host cell may be able to grow in culture without the presence of animal-derived products. The transfected host cell may also be cultured to give a cell line.

The disclosure also provides a method for preparing one or more nucleic acid molecules (e.g., heavy and light chain genes) that encode an antibody of interest, comprising the steps of: (i) preparing an immortalized B cell clone or culturing plasma cells according to the disclosure; (ii) obtaining from the B cell clone or the cultured plasma cells nucleic acid that encodes the antibody of interest. Further, the disclosure provides a method for obtaining a nucleic acid sequence that encodes an antibody of interest, comprising the steps of: (i) preparing an immortalized B cell clone or culturing plasma cells according to the disclosure; (ii) sequencing nucleic acid from the B cell clone or the cultured plasma cells that encodes the antibody of interest.

The disclosure further provides a method of preparing nucleic acid molecule(s) that encode an antibody of interest, comprising the step of obtaining the nucleic acid that was obtained from a transformed B cell clone or cultured plasma cells of the disclosure. Thus the procedures for first obtaining the B cell clone or the cultured plasma cell, and then obtaining nucleic acid(s) from the B cell clone or the cultured plasma cells can be performed at different times by different people in different places (e.g., in different countries).

The disclosure also comprises a method for preparing an antibody (e.g., for pharmaceutical use) according to the present disclosure, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The disclosure also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

Pharmaceutical Compositions

The present disclosure provides pharmaceutical compositions (the terms “pharmaceutical composition” and “antibody composition” are used herein interchangeably) comprising an antibody that neutralizes influenza A and a pharmaceutically acceptable, aqueous vehicle. A vehicle is typically understood to be a material that is suitable for storing, transporting, formulating and/or administering a compound, such as a pharmaceutically active compound, in particular the antibodies according to the present disclosure. For example, the vehicle may be a physiologically acceptable liquid, which is suitable for storing, transporting, and/or administering a pharmaceutically active compound, in particular the antibodies according to the present disclosure.

The pharmaceutical compositions described herein are prepared for injection or infusion into a patient (also referred-to herein as a subject, including in the context of prophylactic administration). In some embodiments, the pharmaceutical composition may be prepared for intravenous, intra-arterial, or intraventricular infusion. In other embodiments, the pharmaceutical composition may be prepared for intravenous, intra-arterial, intraventricular, intramedullary, intraperitoneal, intrathecal, intraventricular, or subcutaneous injection. In particular embodiments, the pharmaceutical composition is prepared for intramuscular (“IM”) injection. In specific embodiments, the pharmaceutical compositions described herein are pharmaceutically acceptable, sterile aqueous solutions exhibiting suitable pH, isotonicity and stability for administration to a human subject. Aqueous vehicles suitable for formulation of the pharmaceutical compositions described herein include water (e.g., sterile water, USP water for injection), as well as isotonic vehicles such as, for example, Sodium Chloride Injection, Ringer's Injection, Lactated Ringer's Injection.

Pharmaceutical compositions according to the present description include an antibody selected from influenza A neutralizing antibodies according to the present description. For example, in some embodiments, pharmaceutical compositions according to the present description include an antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S (according to EU numbering) in the constant region of the heavy chain. In other embodiments, pharmaceutical compositions according to the present description include an antibody comprising a heavy chain variable region comprising an amino acid sequence having 70% or more (i.e. 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having 70% or more identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are maintained. In other embodiments, pharmaceutical compositions according to the present description include an antibody comprising a heavy chain variable region comprising an amino acid sequence having 75% or more (i.e., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising an amino acid sequence having 75% or more identity to SEQ ID NO: 8, wherein the CDR sequences as defined above are maintained. In still other embodiments, pharmaceutical compositions according to the present description include an antibody comprising a heavy chain variable region comprising an amino acid sequence having 80% or more (i.e. 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising an amino acid sequence having 80% or more identity to SEQ ID NO: 8, wherein the CDR sequences as defined above are maintained. In still other embodiments, pharmaceutical compositions according to the present description include an antibody comprising a heavy chain variable region comprising a heavy chain variable region comprising an amino acid sequence having 85% or more (i.e. 85%, 86%, 87%, 88%, 89%, 90%, 91%, 92%, 93%, 94%, 95%, 96%, 97%, 98%, 99% or more) identity to SEQ ID NO: 7 and a light chain variable region comprising an amino acid sequence having 85% or more identity to SEQ ID NO: 8, wherein the CDR sequences as defined above are maintained. In still other embodiments, pharmaceutical compositions according to the present description include an (isolated) antibody comprising a heavy chain variable region comprising a heavy chain variable region comprising an amino acid sequence having 90% or more (i.e. 90%, 91%, 92%, 93%, 94%, 95%, 96%, 97%, 98%, 99% or more) identity to SEQ ID NO: 7 and a light chain variable region comprising an amino acid sequence having 90% or more identity to SEQ ID NO: 8, wherein the CDR sequences as defined above are maintained. In some embodiments, the antibody of the disclosure comprises a heavy chain variable region comprising an amino acid sequence having 95% or more (i.e. 95%. 96%, 97%, 98%, 99% or more) identity to SEQ ID NO: 7 and a light chain variable region comprising an amino acid sequence having 95% or more identity to SEQ ID NO: 8, wherein the CDR sequences as defined above are maintained. In further embodiments, pharmaceutical compositions according to the present description include an antibody comprising a heavy chain variable region comprising an amino acid sequence as set forth in SEQ ID NO: 7 and a light chain variable region comprising the amino acid sequence as set forth in SEQ ID NO: 8, wherein the CDR sequences as defined above are maintained.

In particular embodiments, pharmaceutical compositions according to the present description include an antibody comprising a light chain amino acid sequence according to SEQ ID NO:10 and a heavy chain amino acid sequence according to SEQ ID NO:9.

In some embodiments, the antibody, or a pharmaceutical composition comprising the antibody, has an in vitro influenza inhibition of infection IC90 of about 2.17 μg/mL.

The pharmaceutical compositions include sufficient antibody material to facilitate administration of a therapeutically effective amount of antibody to a patient. In some embodiments, the antibody is included at a concentration selected from 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, and 200 mg/mL. In other embodiments, the antibody is included in the pharmaceutical composition at a concentration selected from above 50 mg/mL, above 75 mg/mL, above 100 mg/mL, above 125 mg/mL, above 150 mg/mL, above 175 mg/mL, above 200 mg/mL, above 225 mg/mL, and above 250 mg/mL. In other embodiments, the pharmaceutical composition comprises the antibody at a concentration in a range from 50 mg/mL to 200 mg/mL, in a range from 75 mg/mL to 225 mg/mL, or in a range from 100 mg/mL to 200 mg/mL. In some embodiments, pharmaceutical composition comprises the antibody at a concentration in a range from 125 mg/ml to 150 mg/ml. In still other embodiments, the pharmaceutical composition comprises the antibody at a concentration of 150 mg/mL.

Pharmaceutical compositions according to the present description may include one or more of a buffer, a surfactant or a coblock polymer, a salt, and a stabilizer (such as a sugar alcohol, disaccharide, or polysaccharide stabilizer, and/or a stabilizing amino acid. In addition, where needed or desired, the pharmaceutical compositions described herein may be formulated to additionally include one or more antioxidant (e.g., ascorbic acid, methionine, ethylenediaminetetraacetic acid (EDTA)).

Pharmaceutical compositions of the disclosure exhibit and maintain a pH that maintains the viability of the antibody, while also being suitable for injection or infusion. The pharmaceutical compositions described herein generally have a pH in a range from about 5.5 to about 6.5, such as in a range from 5.5 to 6.5. In some embodiments, the pharmaceutical composition has a pH in a range from 5.8 to 6.2, for example, about 6.0. In certain embodiments, the pH may be 5.5, 5.6, 5.7, 5.8, 5.9, 6.0, 6.1, 6.2, 6.3, 6.4, or 6.5.

The pharmaceutical composition may include a buffering agent to achieve and maintain a desired pH. Buffers suitable for use in the pharmaceutical compositions described herein include, e.g., acetate, citrate, histidine, succinate, phosphate, and hydroxymethylaminomethane (Tris) buffers. In particular embodiments, the pharmaceutical composition includes a buffer selected from a histidine buffer and a phosphate buffer. In certain embodiments, histidine may be included in the composition at a concentration in a range from 10 mM to 40 mM, or in a range from 20 mM to 40 mM. For example, in specific embodiments, the pharmaceutical composition according to the present description includes histidine at a concentration selected from 10 mM, 15 mM, 20 mM, 25 mM, 30 mM, 35 mM, or 40 mM. In further embodiments, the pharmaceutical composition comprises the antibody at a concentration in a range from 120 mg/mL to 160 mg/mL (e.g., 120 mg/mL, 125 mg/mL, 130 mg/mL, 135 mg/mL, 140 mg/mL, 145 mg/mL, 150 mg/mL, 155 mg/mL, or 160 mg/mL) and the pharmaceutical composition and comprises histidine at a concentration of 20 mM, 25 mM, 30 mM, 35 mM, or 40 mM. In other embodiments, the pharmaceutical composition comprises the antibody at a concentration of about 75 mg/mL and comprises histidine at a concentration of about 10 mM. In certain embodiments, a presently disclosed pharmaceutical composition comprising a histidine buffer has a pH from 5.5 to 6.5, preferably from 5.8 to 6.2, such as 6. In specific embodiments, the pharmaceutical composition has a pH of 6 and includes a histidine buffer.

The pharmaceutical compositions described herein may also include a surfactant or a triblock copolymer. Surfactants, sometimes referred to as “detergents, ” can serve one or more functions. For instance, in aqueous antibody solutions, surfactants and serve to preserve antibody functionality, aid in dissolution of the antibody or other excipients, and/or work to control microbial growth. Surfactants that may be used in the pharmaceutical compositions described herein include, e.g., polysorbate 80 (Tween 80), polysorbate 20 (Tween 20), and poloxamer 188. In some embodiments, the pharmaceutical composition includes a surfactant in a range from 0.01% to 0.05% (w/v) (e.g., 0.01%, 0.02%, 0.03%, 0.04%, or 0.05%). In such embodiments, the surfactant may be selected from polysorbate 80 (Tween 80), polysorbate 20 (Tween 20), and poloxamer 188. In specific embodiments, the pharmaceutical composition of the present description includes polysorbate 80 (Tween 80) or poloxamer 188 in a range from 0.01% to 0.05% (w/v). In other embodiments, the pharmaceutical composition of the present description includes polysorbate 80 (Tween 80) or poloxamer 188 at 0.02% (w/v).

Where the pharmaceutical compositions according to the present disclosure include a sugar alcohol, disaccharide, or polysaccharide stabilizer, the stabilizer may be selected from, e.g., mannitol, sorbitol, sucrose, trehalose, and dextran 40. In particular embodiments, the stabilizer is a disaccharide. In certain embodiments, the pharmaceutical composition includes a disaccharide at in a range from 3.0% to 9.0% (w/v), preferably in a range from 3.6% to 8.6%, more preferably in a range from 4% to 6%, such as in a range from 4.3% to 6.3%, such as 5.3%. In certain such embodiments, the disaccharide is sucrose. In some embodiments, the pharmaceutical composition includes sucrose at 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, or 9.0% (w/v), or is within a range bounded by and including any two of these values. In certain embodiments, the pharmaceutical composition includes sucrose at 5.3% (w/v).

In some embodiments, the pharmaceutical compositions are adapted for administration to mammalian, e.g., human subjects. In such embodiments, the pharmaceutical composition is sterile, and may be specifically prepared to be pyrogen free. In addition, the pharmaceutical composition may be isotonic with respect to humans.

In some embodiments, the pharmaceutical compositions do not have a pH of less than 5.5. In certain further embodiments, the pharmaceutical compositions do not comprise acetate or citrate. In certain embodiments, the pharmaceutical compositions do not comprise a salt, such as, in particular embodiments, NaCl. In certain embodiments, the pharmaceutical compositions do not have a high ionic strength. In certain embodiments, the pharmaceutical compositions do not (i) comprise a phosphate buffer and (ii) have a pH of 7 or higher.

The pharmaceutical compositions described herein may be prepared for direct administration to a subject (i.e., without a reconstitution or mixing step), or they may be prepared as a lyophilized material to be reconstituted in an aqueous vehicle prior to injection or infusion to a subject. For direct administration to a subject, the pharmaceutical composition according to the present disclosure may be provided, e.g., in a pre-filled syringe, or in a vial, such as a glass vial. In some embodiments pharmaceutical compositions of the disclosure are supplied in hermetically-sealed containers. In some embodiments, the pharmaceutical composition may be in kit form, designed such that a combined composition is reconstituted just prior to administration to a subject. For example, a lyophilized antibody may be provided in kit form with sterile water or a sterile buffer.

Medical Treatments and Uses

In a further aspect, the present disclosure provides the use of a pharmaceutical composition according to the present disclosure in prophylaxis and/or treatment of infection with influenza A virus. In particular embodiments, the present disclosure provides methods for prophylaxis and/or treatment of infection with influenza A virus, with the methods comprising: administering to a subject in need thereof, a therapeutically effective amount of a pharmaceutical composition according to the present disclosure.

In some embodiments, various pharmacokinetic (“PK”) parameters are used to describe or characterize the methods and pharmaceutical compositions provided herein. In the context of the present disclosure, the PK parameters referenced in connection with the methods provided herein are derived using standard noncompartmental method in WinNonlin® 8.2 (Certara L.P., Princeton, N.J.). Additional details regarding collection of antibody serum concentrations for purpose of evaluating PK parameters are described in association with the human clinical study referenced in Example 7. The term “t_{1/2}” refers to the elimination half-life of the antibody included in the pharmaceutical composition administered to a subject. The term “Cₗₐₛₜ” generally refers to the last measurable plasma concentration (i.e., subsequent thereto, the substance is not present at a measurable concentration in plasma). In the context of the present description, “Cₗₐₛₜ” refers to the plasma concentration measured on day 140 post administration of the pharmaceutical composition.

Prophylaxis of infection with influenza A virus refers in particular to prophylactic settings, wherein the subject was not diagnosed with infection with influenza A virus (either no diagnosis was performed or diagnosis results were negative) and/or the subject does not show or experience symptoms of infection with influenza A virus. Prophylaxis of infection with influenza A virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza A virus is also particularly useful in subjects at greater risk acquiring influenza A virus infection, e.g., due to increased exposure, for example subjects working or staying in public areas, in particular health care workers.

In therapeutic settings, in contrast, the subject is typically infected with influenza A virus, diagnosed with influenza A virus infection, and/or showing symptoms of influenza A virus infection. Of note, the terms “treatment” and “therapy”/“therapeutic” of influenza A virus infection include (complete) cure as well as attenuation/reduction of influenza A virus infection and/or related symptoms (e.g., attenuation/reduction of severity of infection and/or symptoms, number of symptoms, duration of infection and/or symptoms, or any combination thereof).

The methods described herein include methods for treating influenza A virus infection in subjects diagnosed with influenza A virus infection or in subjects showing symptoms of influenza A virus infection, wherein the methods comprise administering to the subject a therapeutically effective amount of a pharmaceutical composition according to the present disclosure. In certain embodiments of methods for treating subjects diagnosed with or showing symptoms of an influenza A, after administration of the pharmaceutical composition, subject experiences (i) a reduced number and/or severity of one or more respiratory symptoms selected from cough, sore throat, rhinorrhea, and congestion, and/or (ii) a reduced number and/or severity of one or more systemic symptoms selected from fever, chills, myalgia, headache, malaise, and fatigue. For purposes of this disclosure and description of the methods provided herein, the term “fever” refers to an oral temperature >38° C. (100.4° F.).

The methods described herein include methods for prophylaxis of infection with influenza A virus in a subject, wherein the methods comprise administering to the subject a therapeutically effective amount of a pharmaceutical composition according to the present disclosure to the subject. In certain embodiments of a method for prophylaxis of infection with influenza A virus, after administration of the pharmaceutical composition, the subject experiences (i) a reduced number and/or severity of one or more respiratory symptoms selected from cough, sore through, rhinorrhea, and congestion, and/or (ii) a reduced number and/or severity of one or more systemic symptoms selected from fever, chills, myalgia, headache, malaise, and fatigue for at least 4 weeks following administration of the pharmaceutical composition. In other embodiments of a method for prophylaxis of infection with influenza A virus, after administration of the pharmaceutical composition the subject experiences (i) reduced number and/or severity of one or more respiratory symptoms selected from cough, sore through, rhinorrhea, and congestion, and/or (ii) reduced number and/or severity of one or more systemic symptoms selected from fever, chills, myalgia, headache, malaise, and fatigue for at least 12 weeks following administration of the pharmaceutical composition. In still other embodiments of a method for prophylaxis of infection with influenza A virus, after administration of the pharmaceutical composition, the subject experiences (i) a reduced number and/or severity of one or more respiratory symptoms selected from cough, sore through, rhinorrhea, and congestion, and/or (ii) a reduced number and/or severity of one or more systemic symptoms selected from fever, chills, myalgia, headache, malaise, and fatigue for at least 20 weeks following administration of the pharmaceutical composition. In some embodiments of the methods for prophylaxis of infection with influenza A virus described herein, administering the pharmaceutical composition comprises only a single seasonal administration of a therapeutically effective amount of the pharmaceutical to the subject. In some embodiments, administration of the pharmaceutical composition provides the subject with systemic exposure of an influenza A neutralizing antibody according to the present disclosure for a period selected from at least 10 weeks, at least 15 weeks, and at least 20 weeks after a single administration.

It will be understood that reference herein to a reduced number and/or severity of symptoms, which reduction results from administration of a presently disclosed pharmaceutical composition, describes a comparison with a reference subject who did not receive a disclosed pharmaceutical composition. A reference subject can be, for example, (i) the same subject during an earlier period of time (e.g., a prior influenza A virus season), (ii) a subject of a same or a similar: age or age group; gender; pregnancy status; chronic medical condition (such as chronic cardiac, pulmonary, renal, metabolic, neurodevelopmental, liver or hematologic diseases) or lack thereof; and/or immunosuppressive condition or lack thereof; or (iii) a typical subject within a population (e.g., local, regional, or national, including of a same or similar age or age range and/or general state of health) during an influenza A virus season. Prophylaxis can be determined by, for example, the failure to develop a diagnosed influenza A infection and/or the lack of symptoms associated with influenza A infection during a part of a full influenza A season, or over a full influenza A season.

In certain embodiments, the methods provided herein include administering a therapeutically effective amount of a pharmaceutical composition according to the present disclosure to a subject at immediate risk of influenza A infection. An immediate risk of influenza A infection typically occurs during an influenza A epidemic. Influenza A viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza A epidemic is high during November, December, January, February and March, while in the southern hemisphere the risk of an influenza A epidemic is high during May, June, July, August and September.

In some embodiments, a pharmaceutical composition according to the present disclosure is used for prophylaxis and/or treatment of infection with influenza A virus, wherein the pharmaceutical composition is administered up to three months before (a possible) influenza A virus infection, up to one month before (a possible) influenza A virus infection, up to two weeks before (a possible) influenza A virus infection, or up to one week before (a possible) influenza A virus infection. In such embodiments, a method for prophylaxis and/or treatment of infection with influenza A virus comprises administering a therapeutically effective amount of the pharmaceutical composition to a subject, wherein the pharmaceutical composition is administered up to six months before (a possible) influenza A virus infection (e.g., including up to six months before an expected or probable exposure to influenza A virus), up to three months before (a possible) influenza A virus infection, up to one month before (a possible) influenza A virus infection, up to two weeks before (a possible) influenza A virus infection, or up to one week before (a possible) influenza A virus infection. In certain other embodiments, a method for prophylaxis and/or treatment of infection with influenza A virus comprises administering a therapeutically effective amount of the pharmaceutical composition to a subject, wherein the pharmaceutical composition is administered up to six days, up to five days, up to four days, up to three days, or up to two days before the first symptoms of influenza A infection occur. Such treatment schedules may be particularly suited to use of the pharmaceutical compositions for prophylaxis of influenza A infection.

In some of the embodiments of the methods described herein, where a pharmaceutical composition according to the present disclosure is used for prophylaxis, a method according to the present disclosure includes administering to the subject a therapeutically effective amount of a pharmaceutical composition according to the present disclosure to the subject within 1-2 months prior to the beginning of an influenza season, or within the first 1-2 months of the influenza season.

In some embodiments of the methods disclosed herein, after the first administration of a pharmaceutical composition according to the present disclosure, one or more subsequent administrations may follow. Such embodiments include, for example, a method that comprises administering a therapeutically effective amount of a pharmaceutical composition according to the present description to a subject at an interval selected from once every two months, once every three months, once every four months, once every five months, and once every six months. Other such embodiments include, for example, a method according to the present disclosure comprises administering a therapeutically effective amount of a pharmaceutical composition according to the present description at an interval selected from once per year and twice per year. In yet other such embodiments, a method according to the present disclosure comprises administering a therapeutically effective amount of a pharmaceutical composition according to the present description at an interval selected from once per year and twice per year or twice per year over a total treatment period selected from for one, two, three, four, five, six, seven, eight, nine, and ten years.

In each of the methods described herein, administering a therapeutically effective amount (whether for purposes of prophylaxis, treatment, or both), comprises administering an amount of the pharmaceutical composition that delivers a therapeutically effective dose of the influenza A neutralizing antibody. In some embodiments, administering a therapeutically effective amount of the pharmaceutical composition involves delivering an antibody dose ranging from about 50 mg to about 3,000 mg. Depending on the concentration of antibody in the pharmaceutical composition, achieving a desired antibody dose may require multiple injections or infusions as part of a single administration. For example, if a subject is to receive an antibody dose of 600 mg, and the pharmaceutical composition is provided in prepared syringe vials containing 2 ml of an aqueous solution containing the influenza A neutralizing antibody at a concentration of 150 mg/ml, administration of a 600 mg dose will require two injections (each syringe vial contains 300 mg of the antibody). Even where multiple injections or infusions are needed to administer a defined dose, the dose is referred to as a “single dose” and the administration is regarded to be a “single administration.” In general, if multiple injections or infusions are needed to administer a single defined dose, the multiple injections or infusions are administered over a period of about 5 minutes or less, about 15 minutes or less, about 30 minutes or less, about 1 hour or less, about 2 hours or less, about 4 hours or less, about 6 hours or less, about 1 day or less, about 1 week or less, or about 1 month or less.

In certain embodiments of the methods described herein, delivering a therapeutically effective dose of an antibody according to the present disclosure includes delivering a single dose to a subject that ranges from about 60 mg to about 2,500 mg of the influenza A neutralizing antibody. In some embodiments, the methods described herein include administering an amount of the pharmaceutical composition to the subject that is sufficient to deliver a single dose selected from a dose of up to 60 mg, a dose of up to 300 mg, a dose of up to 1,200 mg, a dose of up to 1,800 mg, a dose of up to 2,000 mg, and a dose of up to 2,500 mg of the influenza A neutralizing antibody. In other embodiments, the methods described herein include delivering an amount of the pharmaceutical composition to the subject that is sufficient to provide a single dose selected from 60 mg, 300 mg, 1,200 mg, and 1,800 mg of the influenza A neutralizing antibody. In still further embodiments, the methods described herein include delivering an amount of the pharmaceutical composition to the subject sufficient to provide a single dose selected from 300 mg, 400 mg, 500 mg, 600 mg, 700 mg, 800 mg, 900 mg, 1,000 mg, 1,100 mg, and 1,200 mg of the influenza A neutralizing antibody.

In particular embodiments of the methods described herein, administration of the pharmaceutical composition provides the subject with systemic exposure of an influenza A neutralizing antibody according to the present disclosure for a period selected from at least 10 weeks, at least 15 weeks, and at least 20 weeks after a single administration. In certain such embodiments, the influenza A neutralizing antibody may exhibit a t_{1/2} in a human subject of greater than 40 days. For example, the methods, pharmaceutical compositions, and influenza A neutralizing antibodies described herein may provide a t_{1/2} of the influenza A neutralizing antibody selected from greater than 45 days, greater than 50 days, greater than 55 days, greater than 60 days, and greater than 65 days.

In still other embodiments, the methods, pharmaceutical compositions, and influenza A neutralizing antibodies described herein may provide a t_{1/2} of the influenza A neutralizing antibody selected from between 40 and 70 days, between 45 and 65 days, and between 50 and 60 days. In still other embodiments, the methods, pharmaceutical compositions, and influenza A neutralizing antibodies described herein may provide a t_{1/2} of the influenza A neutralizing antibody selected from any one of 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, and 68 days.

The methods of prophylaxis and/or treatment of infection with influenza A virus described herein may include administering an amount of the pharmaceutical composition sufficient to deliver a single dose of 60 mg of the influenza A neutralizing antibody to the subject. In certain such embodiments, delivery of the single 60 mg dose of the influenza A neutralizing antibody provides systemic exposure of the antibody within the subject over an extended period of time such that the antibody is present in within the subject at a serum concentration from about 1 μg/mL to about 4.5 μg/mL for up to 140 days following administration.

The methods of prophylaxis and/or treatment of infection with influenza A virus described herein may include administering an amount of the pharmaceutical composition sufficient to deliver a single dose of 300 mg of the influenza A neutralizing antibody to the subject. In certain such embodiments, delivery of the single 300 mg dose of the influenza A neutralizing antibody provides systemic exposure of the antibody within the subject over an extended period of time such that the antibody is present in within the subject at a serum concentration from about 5 μg/mL to about 26.5 μg/mL for up to 140 days following administration.

The methods of prophylaxis and/or treatment of infection with influenza A virus described herein may include administering an amount of the pharmaceutical composition sufficient to deliver a single dose of 1,200 mg of the influenza A neutralizing antibody to the subject. In certain such embodiments, delivery of the single 1,200 mg dose of the influenza A neutralizing antibody provides systemic exposure of the antibody within the subject over an extended period of time such that the antibody is present in within the subject at a serum concentration from about 27 μg/mL to about 110 μg/mL for up to 140 days following administration.

The methods of prophylaxis and/or treatment of infection with influenza A virus described herein may include administering an amount of the pharmaceutical composition sufficient to deliver a single dose of 1,800 mg of the influenza A neutralizing antibody to the subject. In certain such embodiments, delivery of the single 1,800 mg dose of the influenza A neutralizing antibody provides systemic exposure of the antibody within the subject over an extended period of time such that the antibody is present in within the subject at a serum concentration from about 33.5 μg/mL to about 150 μg/mL for up to 140 days following administration

In any of the methods for prophylaxis and/or treatment of infection with influenza A virus described herein, the pharmaceutical composition can be administered via injection or infusion. When administered by infusion, the pharmaceutical compositions may be administered by, e.g., intravenous, intra-arterial, or intraventricular infusion. When administered by injection, the pharmaceutical compositions may be administered by, e.g., intravenous, intra-arterial, intraventricular, intramedullary, intraperitoneal, intrathecal, intraventricular, or subcutaneous injection. In specific embodiments of the methods described herein, the pharmaceutical composition is administered via intramuscular (“IM”) injection.

In particular embodiments of the methods for prophylaxis and/or treatment of infection with influenza A virus described herein, the pharmaceutical composition and influenza A neutralizing antibody are well-tolerated by the subject when administered in the amounts and doses described herein. For example, in certain embodiments, the methods for prophylaxis and/or treatment of infection with influenza A virus described herein do not result in the subject experiencing an adverse event (AE), according to the Common Terminology Criteria for Adverse Events (CTCAE). In other embodiments, the methods for prophylaxis and/or treatment of infection with influenza A virus described herein do not result in the subject experiencing a moderate adverse event (AE), according to the Common Terminology Criteria for Adverse Events (CTCAE). In still other embodiments, the methods for prophylaxis and/or treatment of infection with influenza A virus described herein do not result in the subject experiencing a serious adverse event (AE), according to the Common Terminology Criteria for Adverse Events (CTCAE).

In particular embodiments of the methods for prophylaxis and/or treatment of infection with influenza, the subject is from 18 years to 65 years of age. In certain such embodiments, the subject has a body mass index selected from a range of 18 kg/m² to 32 kg/m² and range of 18 kg/m² to 35 kg/m².

Combination Therapy

The administration a pharmaceutical composition according to the present disclosure in the methods and uses according to the disclosure can be carried out alone or in combination with a co-agent (also referred to as “additional active component” herein), which may be useful for preventing and/or treating influenza infection.

The disclosure encompasses the administration of a pharmaceutical composition according to the present disclosure, wherein it is administered to a subject prior to, simultaneously with or after a co-agent or another therapeutic regimen useful for treating and/or preventing influenza. Said pharmaceutical composition administered in combination with said co-agent can be administered in the same or different composition(s) and by the same or different route(s) of administration. As used herein, expressions like “combination therapy”, “combined administration”, “administered in combination” and the like refer to a combined action of the drugs (which are to be administered “in combination”). To this end, the combined drugs are usually present at a site of action at the same time and/or within an overlapping time window. It may also be possible that the effects resulting from one of the drugs are still ongoing (even if the drug itself may no longer be present at a detectable) while the other drug is administered, such that effects of both drugs can interact. However, a drug which was administered long before another drug (e.g., more than one, two, three or more months or a year), such that it is no longer present at a detectable level (or its effects are not ongoing) when the other drug is administered, is typically not considered to be administered “in combination”. For example, influenza medications administered in distinct (e.g., successive) influenza seasons are usually not administered “in combination”.

Said other therapeutic regimens or co-agents may be, for example, an antiviral. An antiviral (or “antiviral agent” or “antiviral drug”) refers to a class of medication used specifically for treating viral infections. Like antibiotics for bacteria, antivirals may be broad spectrum antivirals useful against various viruses or specific antivirals that are used for specific viruses. Unlike most antibiotics, antiviral drugs do usually not destroy their target pathogen; instead they typically inhibit their development.

Thus, in another aspect of the present disclosure a pharmaceutical composition according to the present disclosure is administered in combination with (prior to, simultaneously or after) an antiviral for the (medical) uses as described herein.

In general, an antiviral may be a broad spectrum antiviral (which is useful against influenza viruses and other viruses) or an influenza virus-specific antiviral. In some embodiments, the antiviral is not an antibody. For example, the antiviral may be a small molecule drug. Examples of small molecule antivirals useful in prophylaxis and/or treatment of influenza are described in Wu X, Wu X, Sun Q, et al. Progress of small molecular inhibitors in the development of anti-influenza virus agents. Theranostics. 2017; 7(4):826-845. As described in Wu et al., 2017, the skilled artisan is familiar with antivirals useful in prophylaxis and/or treatment of influenza. Further antivirals useful in influenza are described in Davidson S. Treating Influenza Infection, From Now and Into the Future. Front Immunol. 2018; 9:1946; and in: Koszalka P, Tilmanis D, Hurt A C. Influenza antivirals currently in late-phase clinical trial. Influenza Other Respir Viruses. 2017; 11(3):240-246.

Antivirals useful in prophylaxis and/or treatment of influenza include (i) agents targeting functional proteins of the influenza virus itself and (ii) agents targeting host cells, e.g. the epithelium.

Host cell targeting agents include the thiazolide class of broad-spectrum antivirals, sialidase fusion proteins, type III interferons, Bcl-2 (B cell lymphoma 2) inhibitors, protease inhibitors, V-ATPase inhibitors and antioxidants. Examples of the thiazolide class of broad-spectrum antivirals include nitazoxanide (NTZ), which is rapidly deacetylated in the blood to the active metabolic form tizoxanide (TIZ), and second generation thiazolide compounds, which are structurally related to NTZ, such as RM5061. Fludase (DAS181) is an example for sialidase fusion proteins. Type III IFNs include, for example, IFNλ. Non-limiting examples of Bcl-2 inhibitors include ABT-737, ABT-263, ABT-199, WEHI-539 and A-1331852 (Davidson S. Treating Influenza Infection, From Now and Into the Future. Front Immunol. 2018; 9:1946). Examples of protease inhibitors include nafamostat, Leupeptin, epsilon-aminocapronic acid, Camostat and Aprotinin. V-ATPase inhibitors include NorakinR, ParkopanR, AntiparkinR and AkinetonR. An example of an antioxidant is alpha-tocopherol.

In some embodiments, the antiviral is an agent targeting a functional protein of the influenza virus itself. For example, the antiviral may target a functional protein of the influenza virus, which is not hemagglutinin. In general, antivirals targeting a functional protein of the influenza virus include entry inhibitors, hemagglutinin inhibitors, neuraminidase inhibitors, influenza polymerase inhibitors (RNA-dependent RNA polymerase (RdRp) inhibitors), nucleocapsid protein inhibitors, M2 ion channel inhibitors and arbidol hydrochloride. Non-limiting examples of entry inhibitors include triterpenoids derivatives, such as glycyrrhizic acid (glycyrrhizin) and glycyrrhetinic acid; saponins; uralsaponins M-Y (such as uralsaponins M); dextran sulphate (DS); silymarin; curcumin; and lysosomotropic agents, such as Concanamycin A, Bafilomycin A1, and Chloroquine. Non-limiting examples of hemagglutinin inhibitors include BMY-27709; stachyflin; natural products, such as Gossypol, Rutin, Quercetin, Xylopine, and Theaflavins; trivalent glycopeptide mimetics, such as compound 1 described in Wu X, Wu X, Sun Q, et al. Progress of small molecular inhibitors in the development of anti-influenza virus agents. Theranostics. 2017; 7(4):826-845; podocarpic acid derivatives, such as compound 2 described in Wu X, Wu X, Sun Q, et al. Progress of small molecular inhibitors in the development of anti-influenza virus agents. Theranostics. 2017; 7(4):826-845; natural product pentacyclic triterpenoids, such as compound 3 described in Wu X, Wu X, Sun Q, et al. Progress of small molecular inhibitors in the development of anti-influenza virus agents. Theranostics. 2017; 7(4):826-845; and prenylated indole diketopiperazine alkaloids, such as Neoechinulin B. Non-limiting examples of nucleocapsid protein inhibitors include nucleozin, Cycloheximide, Naproxen and Ingavirin. Non-limiting examples of M2 ion channel inhibitors include the approved M2 inhibitors Amantadine and Rimantadine and derivatives thereof; as well as non-adamantane derivatives, such as Spermine, Spermidine, Spiropiperidine and pinanamine derivatives.

In some embodiments, the antiviral is selected from neuraminidase (NA) inhibitors and influenza polymerase inhibitors (RNA-dependent RNA polymerase (RdRp) inhibitors). Non-limiting examples of neuraminidase (NA) inhibitors include zanamivir; oseltamivir; peramivir; laninamivir; derivatives thereof such as compounds 4-10 described in Wu X, Wu X, Sun Q, et al. Progress of small molecular inhibitors in the development of anti-influenza virus agents. Theranostics. 2017; 7(4):826-845, and dimeric zanamivir conjugates (e.g., as described in Wu X, Wu X, Sun Q, et al. Progress of small molecular inhibitors in the development of anti-influenza virus agents. Theranostics. 2017; 7(4):826-845); benzoic acid derivatives (e.g., as described in Wu X, Wu X, Sun Q, et al. Progress of small molecular inhibitors in the development of anti-influenza virus agents. Theranostics. 2017; 7(4):826-845; such as compounds 11-14); pyrrolidine derivatives (e.g., as described in Wu X, Wu X, Sun Q, et al. Progress of small molecular inhibitors in the development of anti-influenza virus agents. Theranostics. 2017; 7(4):826-845; such as compounds 15-18); ginkgetin-sialic acid conjugates; flavanones and flavonoids isoscutellarein and its derivatives (e.g., as described in Wu X, Wu X, Sun Q, et al. Progress of small molecular inhibitors in the development of anti-influenza virus agents. Theranostics. 2017; 7(4) : 826-845); AV5080; and N-substituted oseltamivir analogues (e.g., as described in Wu X, Wu X, Sun Q, et al. Progress of small molecular inhibitors in the development of anti-influenza virus agents. Theranostics. 2017; 7(4):826-845). Non-limiting examples of influenza polymerase inhibitors (RNA-dependent RNA polymerase (RdRp)) inhibitors include RdRp disrupting compounds, such as those described in Wu X, Wu X, Sun Q, et al. Progress of small molecular inhibitors in the development of anti-influenza virus agents. Theranostics. 2017; 7(4):826-845; PB2 cap-binding inhibitors, such as JNJ63623872 (VX-787); cap-dependent endonuclease inhibitors, such as baloxavir marboxil (S-033188); PA endonuclease inhibitors, such as AL-794, EGCG and its aliphatic analogues, N-hydroxamic acids and N-hydroxyimides, flutimide and its aromatic analogues, tetramic acid derivatives, L-742,001, ANA-0, polyphenolic catechins, phenethyl-phenylphthalimide analogues, macrocyclic bisbibenzyls, pyrimidinoles, fullerenes, hydroxyquinolinones, hydroxypyridinones, hydroxypyridazinones, trihydroxy-phenyl-bearing compounds, 2-hydroxy-benzamides, hydroxy-pyrimidinones, β-diketo acid and its bioisosteric compounds, thiosemicarbazones, bisdihydroxyindole-carboxamides, and pyrido-piperazinediones (Endo-1); and nucleoside and nucleobase analogue inhibitors, such as ribavirin, favipiravir (T-705), 2′-Deoxy-2′-fluoroguanosine (2′-FdG), 2′-substituted carba-nucleoside analogues, 6-methyl-7-substituted-7-deaza purine nucleoside analogues, and 2′-deoxy-2′-fluorocytidine (2′-FdC). For example, the antiviral may be zanamivir, oseltamivir or baloxavir.

Thus, a pharmaceutical composition according to the present disclosure may comprise one or more of the additional active components. An influenza A neutralizing antibody according to the present disclosure can be present in the same pharmaceutical composition as the additional active component (co-agent). Alternatively, an influenza A neutralizing antibody according to the present disclosure and the additional active component (co-agent) are comprised in distinct pharmaceutical compositions (e.g., not in the same composition). Accordingly, if more than one additional active component (co-agent) is envisaged, each additional active component (co-agent) and an antibody, or an antigen binding fragment, according to the present disclosure may be comprised by a different pharmaceutical composition. Such different pharmaceutical compositions may be administered either combined/simultaneously or at separate times and/or by separate routes of administration.

An influenza A neutralizing antibody according to the present disclosure and the additional active component (co-agent) may provide an additive or a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Accordingly, the present disclosure also provides a combination of (i) an influenza A neutralizing antibody as described herein, and (ii) an antiviral agent as described above.

The present disclosure includes the following exemplary embodiments.

Embodiment 1. An antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S in the constant region of the heavy chain.

Embodiment 2. The antibody of Embodiment 1, wherein the antibody binds to hemagglutinin of an influenza A virus.

Embodiment 3. The antibody of Embodiment 1 or 2, wherein the antibody neutralizes infection with an influenza A virus.

Embodiment 4. The antibody of Embodiment 3, wherein the antibody neutralizes influenza A infection at a dose, which does not exceed half of the dose required for neutralization of influenza A with a comparative antibody, which differs from said antibody only in that it does not contain the mutations M428L and N434S in the constant region of the heavy chain.

Embodiment 5. The antibody of Embodiment 4, wherein the dose does not exceed one third of the dose required for neutralization of influenza A with said comparative antibody.

Embodiment 6. The antibody of Embodiment 4 or 5, wherein the dose does not exceed one fifth of the dose required for neutralization of influenza A with said comparative antibody.

Embodiment 7. The antibody of any one of the previous Embodiments, wherein the antibody is a human antibody.

Embodiment 8. The antibody of any one of the previous Embodiments, wherein the antibody is a monoclonal antibody.

Embodiment 9. The antibody of any one of the previous Embodiments, wherein the antibody is of the IgG type.

Embodiment 10. The antibody of Embodiment 6, wherein the antibody is of the IgG1 type.

Embodiment 11. The antibody of any one of the previous Embodiments, wherein the light chain of the antibody is a kappa light chain.

Embodiment 12. The antibody of any one of the previous Embodiments, wherein the antibody comprises a heavy chain variable region comprising an amino acid sequence having at least 70% identity to SEQ ID NO: 7 and a light chain variable region comprising the amino acid sequence having at least 70% identity to SEQ ID NO: 8, wherein the CDR sequences as defined in Embodiment 1 are maintained.

Embodiment 13. The antibody of any one of the previous Embodiments, wherein the antibody comprises a heavy chain variable region comprising an amino acid sequence having at least 75% identity to SEQ ID NO: 7 and a light chain variable region comprising the amino acid sequence having at least 75% identity to SEQ ID NO: 8, wherein the CDR sequences as defined in Embodiment 1 are maintained.

Embodiment 14. The antibody of any one of the previous Embodiments, wherein the antibody comprises a heavy chain variable region comprising an amino acid sequence having at least 80% identity to SEQ ID NO: 7 and a light chain variable region comprising the amino acid sequence having at least 80% identity to SEQ ID NO: 8, wherein the CDR sequences as defined in Embodiment 1 are maintained.

Embodiment 15. The antibody of any one of the previous Embodiments, wherein the antibody comprises a heavy chain variable region comprising an amino acid sequence having at least 85% identity to SEQ ID NO: 7 and a light chain variable region comprising the amino acid sequence having at least 85% identity to SEQ ID NO: 8, wherein the CDR sequences as defined in Embodiment 1 are maintained.

Embodiment 16. The antibody of any one of the previous Embodiments, wherein the antibody comprises a heavy chain variable region comprising an amino acid sequence having at least 90% identity to SEQ ID NO: 7 and a light chain variable region comprising the amino acid sequence having at least 90% identity to SEQ ID NO: 8, wherein the CDR sequences as defined in Embodiment 1 are maintained.

Embodiment 17. The antibody of any one of the previous Embodiments, wherein the antibody comprises a heavy chain variable region comprising an amino acid sequence having at least 95% identity to SEQ ID NO: 7 and a light chain variable region comprising the amino acid sequence having at least 95% identity to SEQ ID NO: 8, wherein the CDR sequences as defined in Embodiment 1 are maintained.

Embodiment 18. The antibody of any one of the previous Embodiments, wherein the antibody comprises a heavy chain variable region comprising an amino acid sequence as set forth in SEQ ID NO: 7 and a light chain variable region comprising the amino acid sequence as set forth in SEQ ID NO: 8, wherein the CDR sequences as defined in Embodiment 1 are maintained.

Embodiment 19. The antibody of any one of the previous Embodiments, wherein the CH3 region of the antibody does not comprise any further mutation in addition to M428L and N434S.

Embodiment 20. The antibody of any one of the previous Embodiments, wherein the Fc region of the antibody does not comprise any further mutation in addition to M428L and N434S.

Embodiment 21. The antibody of any one of the previous Embodiments, wherein the antibody comprises a heavy chain comprising an amino acid sequence as set forth in SEQ ID NO: 9 and a light chain comprising an amino acid sequence as set forth in SEQ ID NO: 10.

Embodiment 22. The antibody of any one of the previous Embodiments, wherein the antibody has a heavy chain consisting of an amino acid sequence as set forth in SEQ ID NO: 9 and a light chain consisting of an amino acid sequence as set forth in SEQ ID NO: 10.

Embodiment 23. The antibody of any one of the previous Embodiments for use in prophylaxis or treatment of infection with influenza A virus, wherein, optionally, the antibody, or a pharmaceutical composition comprising the antibody, has an in vitro influenza inhibition of infection IC90 of about 2.17 μg/mL.

Embodiment 24. The antibody for use according to Embodiment 23, wherein the antibody is administered prophylactically.

Embodiment 25. The antibody for use according to any one of Embodiments 23 or 24, wherein the subject to be treated is at immediate risk of influenza A infection.

Embodiment 26. A nucleic acid molecule comprising a polynucleotide encoding the antibody of any one of Embodiments 1-22.

Embodiment 27. A vector comprising the nucleic acid molecule of Embodiment 26.

Embodiment 28. A cell expressing the antibody of any one of Embodiments 1-22, or comprising the vector of Embodiment 27.

Embodiment 29. A pharmaceutical composition comprising the antibody of any one of Embodiments 1-22, the nucleic acid of Embodiment 26, the vector of Embodiment 27, or the cell of Embodiment 28, and, optionally, a pharmaceutically acceptable diluent or carrier.

Embodiment 30. The pharmaceutical composition of Embodiment 29, comprising the antibody at 150 mg/mL.

Embodiment 31. The pharmaceutical composition of Embodiment 30 or 31, further comprising water (e.g., USP water for injection, or US sterile water for injection).

Embodiment 32. The pharmaceutical composition of any one of Embodiments 38-40, further comprising histidine, optionally at a concentration of 10 mM to 40 mM, preferably 20 mM, in the pharmaceutical composition.

Embodiment 33. The pharmaceutical composition of any one of Embodiments 29-32, further comprising a sugar, such as a disaccharide, such as sucrose, optionally in a range from 3.0% to 9.0% (w/v), preferably in a range from 3.6% to 8.6%, more preferably in a range from 4% to 6%.

Embodiment 34. The pharmaceutical composition of any one of Embodiments 29-33, further comprising a surfacant or a triblock copolymer, optionally a polysorbate or poloxamer 188, preferably polysorbate 80 (PS80), optionally in a range from 0.01% to 0.05% (w/v), preferably 0.02% (w/v).

Embodiment 35. The pharmaceutical composition of any one of Embodiments 29-34, wherein the pharmaceutical composition has a pH in a range from 5.5 to 6.5, or in a range from 5.8 to 6.2, or a pH of 5.5, 5.6, 5.7, 5.8, 5.9, 6.0, 6.1, 6.2, 6.3, 6.4, or 6.5, preferably of 6.0.

Embodiment 36. Use of the antibody of any one of Embodiments 1-22, the nucleic acid of Embodiment 26, the vector of Embodiment 27, the cell of Embodiment 28 or the pharmaceutical composition of any one of Embodiments 29-35 in the manufacture of a medicament for prophylaxis, treatment or attenuation of influenza A virus infection.

Embodiment 37. The antibody of any one of Embodiments 1-22, the nucleic acid of Embodiment 26, the vector of Embodiment 27, the cell of Embodiment 28 or the pharmaceutical composition of any one of Embodiments 29-35 for use in prophylaxis or treatment of infection with influenza A virus.

Embodiment 38. The antibody, the nucleic acid, the vector, the cell or the pharmaceutical composition for use according to Embodiment 37, wherein the antibody, the nucleic acid, the vector, the cell or the pharmaceutical composition is administered prophylactically.

Embodiment 39. The antibody, the nucleic acid, the vector, the cell or the pharmaceutical composition for use according to Embodiment 37 or Embodiment 38, wherein the antibody, the nucleic acid, the vector, the cell or the composition is administered in combination with an antiviral.

Embodiment 40. The antibody, the nucleic acid, the vector, the cell or the pharmaceutical composition for use according to Embodiment 39, wherein the antiviral is selected from neuraminidase inhibitors and influenza polymerase inhibitors.

Embodiment 41. The antibody, the nucleic acid, the vector, the cell or the pharmaceutical composition for use according to Embodiment 39 or 40, wherein the antiviral is selected from oseltamivir, zanamivir and baloxavir.

Embodiment 42. A combination of
(i) the antibody of any one of Embodiments 1-22, and
(ii) an antiviral agent.

Embodiment 43. The combination of Embodiment 42, wherein the antiviral is selected from neuraminidase inhibitors and influenza polymerase inhibitors.

Embodiment 44. The combination of Embodiment 42 or 43, wherein the antiviral is selected from oseltamivir, zanamivir and baloxavir.

Embodiment 45. The combination of any one of Embodiments 42-44 for use in prophylaxis or treatment of infection with influenza A virus.

Embodiment 46. A method of reducing influenza A virus infection, or lowering the risk of influenza A virus infection, comprising: administering to a subject in need thereof, a therapeutically effective amount of the antibody of any one of Embodiments 1-22.

Embodiment 47. The method of Embodiment 46, wherein the antibody is administered prophylactically.

Embodiment 48. The method of any Embodiment 46 or 47, wherein said subject is at immediate risk of influenza A infection.

Embodiment 49. The method of any one of Embodiments 46-48, wherein the antibody is administered in combination with an antiviral.

Embodiment 50. A method of treating or preventing an Influenza A infection in a subject, the method comprising administering to the subject a single dose of a pharmaceutical composition comprising the antibody of any one of Embodiments 1-22, wherein, optionally, the antibody comprises a light chain amino acid sequence according to SEQ ID NO:10 and a heavy chain amino acid sequence according to SEQ ID NO:9.

Embodiment 51. The method of any one of Embodiment 50, wherein the pharmaceutical composition comprises the antibody at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

Embodiment 52. The method of Embodiment 50 or 51, wherein the single dose comprises 3, 4, 5, 6, or 7, preferably 5, mg of the antibody per kg of the subject's body weight.

Embodiment 53. The method of any one of Embodiments 50-52, wherein the single dose comprises up to 60 mg, up to 300 mg, up to 1200 mg, up to 1800 mg, or up to 3000 mg of the antibody.

Embodiment 54. The method of any one of Embodiments 50-53, wherein the antibody is administered at a dose of 60 mg, 300, 1200, or 1800 mg.

Embodiment 55. The method of any one of Embodiments 50-54, wherein the antibody is administered at a dose of 300, 400, 500, 600, 700, 800, 900, 1100, or 1200 mg.

Embodiment 56. The method of any one of Embodiments 50-55, wherein the subject is human.

Embodiment 57. The method of any one of Embodiments 50-56, wherein the method comprises intramuscular (IM) injection.

Embodiment 58. The method of any one of Embodiments 50-57, wherein the pharmaceutical composition further comprises water (e.g., USP water for injection, or US sterile water for injection).

Embodiment 59. The method of any one of Embodiments 50-58, wherein the pharmaceutical composition further comprises histidine, optionally at a concentration of 10 mM to 40 mM, preferably 20 mM, in the composition.

Embodiment 60. The method of any one of Embodiments 50-59, wherein the pharmaceutical composition further comprises a sugar, such as a disaccharide, such as sucrose, optionally in a range from 3.0% to 9.0% (w/v), preferably in a range from 3.6% to 8.6%, more preferably in a range from 4% to 6%.

Embodiment 61. The method of any one of Embodiments 50-60, wherein the pharmaceutical composition further comprises a surfactant or a triblock copolymer, optionally a polysorbate or poloxamer 188, preferably polysorbate 80 (PS80), optionally in a range from 0.01% to 0.05% (w/v), preferably 0.02% (w/v).

Embodiment 62. The method of any one of Embodiments 50-61, wherein the pharmaceutical composition has a pH in a range from 5.8 to 6.2, in a range from 5.9 to 6.1, or of 5.8, of 5.9, of 6.0, of 6.1, or of 6.2.

Embodiment 63. The method of any one of Embodiments 50-62, wherein the single dose comprises or consists of from 0.8 mL to 4 mL of the pharmaceutical composition per injection.

Embodiment 64. The method of Embodiment 63, wherein the single dose comprises or consists of 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.1 mL, 3.2 mL, 3.3 mL, 3.4 mL, 3.5 mL, 3.6 mL, 3.7 mL, 3.8 mL, 3.9 mL, or 4.0 mL of the pharmaceutical composition, per injection.

Embodiment 65. The method of any one of Embodiments 50-64, wherein at about 4 weeks, at about 12 weeks, and/or about 20 weeks following administering the pharmaceutical composition to the subject, the subject: (i) has a reduced number and/or severity of a respiratory symptom selected from: cough, sore throat;, rhinorrhea; congestion; or any combination thereof, and/or (ii) has a reduced number and/or severity of a systemic symptom selected from: fever [oral temperature >38° C. (100.4° F.)]; chills; myalgia; headache; malaise; fatigue; or any combination thereof, as compared to a reference subject (e.g., a subject of a same gender, age, body weight, and/or general health) over a same time period who received a placebo or did not receive a therapy or vaccine for influenza A.

Embodiment 66. The method of any one of Embodiments 50-67, wherein the subject is from 18 years to 65 years of age and/or has a body mass index in a range from 18 kg/m² to 32 kg/m² or in a range from 18 kg/m² to 35 kg/m².

Embodiment 67. The method of any one of Embodiments 50-66, wherein: (i) the single dose comprises 300 mg of the antibody, wherein the pharmaceutical composition comprises the antibody at 150 mg/mL, and the dose is administered by a single injection comprising 2 mL of the pharmaceutical composition; (ii) the single dose comprises 1200 mg of the antibody, wherein the pharmaceutical composition comprises the antibody at 150 mg/mL, and the dose is administered by two injections each comprising 4 mL of the composition; (iii) the single dose comprises 1800 mg of the antibody, wherein the pharmaceutical composition comprises the antibody at 150 mg/mL, and the dose is administered by three injections each comprising 4 mL of the pharmaceutical composition; or (iv) the single dose comprises 60 mg of the antibody, wherein the pharmaceutical composition comprises antibody at 150 mg/mL, and the dose is administered by one injection comprising 0.4 mL of the pharmaceutical composition.

Embodiment 68. The method of any one of Embodiments 50-67, comprising administering the pharmaceutical composition once to the subject during a six-month period.

Embodiment 69. The method of any one of Embodiments 50-68, comprising administering a single dose comprising the pharmaceutical composition once to the subject during a twelve-month period.

Embodiment 70. The method of any one of Embodiments 50-68, comprising administering the pharmaceutical composition twice to the subject during a six-month period, such as once every three months.

Embodiment 71. The method of any one of Embodiments 50-70, comprising administering the pharmaceutical composition within 1-2 months prior to the beginning of an influenza season (e.g., in the United States, a flu season can begin in October, in November, or in December), or within the first 1-2 months of the influenza season.

Embodiment 72. The method of any one of Embodiments 50-71, wherein the antibody, or the pharmaceutical composition comprising the antibody, has an in vitro influenza inhibition of infection IC90 of about 2.17 μg/mL.

Embodiment 73. The method of any one of Embodiments 50-72, comprising administering the subject a single dose of the pharmaceutical composition, wherein the composition comprises the antibody at 150 mg/mL, and the dose is administered by a single injection comprising 2 mL of the pharmaceutical composition, and wherein: (i) the administered pharmaceutical composition comprises about 60 mg of the antibody, and the antibody is present in serum from the subject at a concentration from about 1 μg/mL to about 7 μg/mL for up to 120 days following administration; (ii) the administered pharmaceutical composition comprises about 300 mg of the antibody, and the antibody is present in serum from the subject at a concentration from about 8 μg/mL to about 20 μg/mL for up to 120 days following administration; (iii) the administered pharmaceutical composition comprises about 1200 mg of the antibody, and the antibody is present in serum from the subject at a concentration from about 50 to μg/mL to about 100 μg/mL for up to 120 days following administration; (iv) the administered pharmaceutical composition comprises about 1800 mg of the antibody, and the antibody is present in serum from the subject at a concentration from about 70 to μg/mL to about 110 μg/mL for up to 120 days following administration; and/or (v) the antibody has an in vivo t_{1/2} in the subject of from 49 to 68 days.

Embodiment 74. The method of any one of Embodiments 50-73, wherein the antibody of the pharmaceutical composition has an in vivo t_{1/2} in a human subject of from 49 to 68 days, such as 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, or 68 days.

Embodiment 75. The method of any one of Embodiments 50-74, wherein the subject does not experience an adverse event (AE), according to the Common Terminology Criteria for Adverse Events (CTCAE), optionally for up to 140 days, after the single dose of the pharmaceutical composition is administered.

Embodiment 76. The method of any one of Embodiments 50-75, wherein the subject does not experience a moderate adverse event (AE), according to the Common Terminology Criteria for Adverse Events (CTCAE), optionally for up to 140 days after the single dose of the pharmaceutical composition is administered.

Embodiment 77. The method of any one of Embodiments 50-76, wherein the subject does not experience a serious adverse event (AE), according to the Common Terminology Criteria for Adverse Events (CTCAE), optionally for up to 140 days after the single dose of the pharmaceutical composition is administered.

Embodiment 78. The method of any one of Embodiments 50-77, wherein: (i) the single dose comprises 300 mg of the antibody, wherein the pharmaceutical composition comprises the antibody at 150 mg/mL, and the single dose comprises a single injection comprising 2 mL of the pharmaceutical composition; (ii) the single dose comprises 1200 mg of the antibody, wherein the pharmaceutical composition comprises the antibody at 150 mg/mL, and the single dose comprises two injections each comprising 4 mL of the pharmaceutical composition; (iii) the single dose comprises 1800 mg of the antibody, wherein the pharmaceutical composition comprises the antibody at 150 mg/mL, and the single dose comprises three injections each comprising 4 mL of the pharmaceutical composition; or (iv) the single dose comprises 60 mg of the antibody, wherein the pharmaceutical composition comprises the antibody at 150 mg/mL, and the single dose comprises 0.4 mL of the composition.

Embodiment 79. A pharmaceutical composition comprising an antibody that comprises a light chain amino acid sequence according to SEQ ID NO:10 and a heavy chain amino acid sequence according to SEQ ID NO:9, wherein the antibody is present in the pharmaceutical composition at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

Embodiment 80. The pharmaceutical composition of Embodiment 79, wherein the pharmaceutical composition further comprises water (e.g., USP water for injection, or US sterile water for injection).

Embodiment 81. The pharmaceutical composition of Embodiment 79 or 80, further comprising histidine, optionally at a concentration of 10 mM to 40 mM, preferably 20 mM, in the composition.

Embodiment 82. The pharmaceutical composition of any one of Embodiments 79-81, further comprising a sugar, such as a disaccharide, such as sucrose, in a range from 3.0% to 9.0% (w/v), preferably from 3.6% to 8.6%, more preferably in a range from 4% to 6%.

Embodiment 83. The pharmaceutical composition of any one of Embodiments 79-82, further comprising a surfactant or a triblock copolymer, optionally a polysorbate or poloxamer 188, preferably polysorbate 80 (PS80), optionally in a range from 0.01% to 0.05% (w/v), preferably 0.02%.

Embodiment 84. The pharmaceutical composition of any one of Embodiments 79-83, wherein the composition has a pH in a range from 5.5 to 6.5, or in a range from 5.8 to 6.2, or a pH of 5.5, 5.6, 5.7, 5.8, 5.9, 6.0, 6.1, 6.2, 6.3, 6.4, or 6.5, preferably of 6.0.

Embodiment 85. A, preferably glass, vial comprising the pharmaceutical composition of any one of Embodiments 79-84.

Embodiment 86. A (e.g., pre-filled) syringe comprising the pharmaceutical composition of any one of Embodiments 79-84.

### BRIEF DESCRIPTION OF THE FIGURES

In the following a brief description of the appended figures will be given. The figures are intended to illustrate the present disclosure in more detail. However, they are not intended to limit the subject matter of the disclosure in any way.

FIG. 1 shows for Example 2 the plasma concentration of human antibodies FluAB_MLNS (open squares) and FluAB_wt (comparative antibody; filled circles) in macaque plasma samples assessed via ELISA until day 56.

FIGS. 2A-2B show for Example 3 plasma concentrations of FluAB_MLNS (animals C90142 (2A), C90190 (2B)) measured using an anti-CH2 antibody ELISA to quantify total human mAb or HA antigen-binding ELISA to determine functionality of the mAbs. Graphs show linear regression between total human mAb quantification and HA binding for individual animals at selected time points (days 1, 21, 56, 86, and 113).

FIGS. 3A-3B show for Example 4 (3A) the concentrations of human antibodies FluAB_MLNS and FluAB_wt in nasal swabs as measured using ELISA and normalized to urea content; and (3B) Biodistribution of human antibodies FluAB_MLNS and FluAB_wt, expressed as % urea-normalized concentration in nasal swabs over plasma concentrations. Individual animal IDs and inoculated human antibody variant (FluAB_MLNS or FluAB_wt) are indicated below.

FIGS. 4A-4E show for Example 5 the cumulative bodyweight change over time in Tg32 mice treated with either FluAB_wt (4B, 4D, circles), FluAB_MLNS (4C, 4E, squares) at 1 mg/kg (4B, 4C, grey symbols) and 0.3 mg/kg (4D, 4E, light gray symbols) or left untreated (4A, triangles); all mice infected intranasally with PR8 virus. Individual animals are shown; the thick black line represents the average trend of BW±SD. The number of individuals per group is indicated. * p<0.05, ** p<0.01, *** p<0.001 vs control alone (A), ^{∘} p<0.05, ^{∘∘} p<0.01, all vs the relative timepoints of MEDI8852, 2-way ANOVA with Bonferroni's multiple test correction.

FIGS. 5A and 5B show for Example 5 the % of survival comparison between 1 mg/kg dose (5A) and 0.3 mg/kg dose (right panel) in infected Tg32 male mice treated with nothing (5B), FluAB_wt, or FluAB_MLNS. ** p<0.01 vs untreated mice (CTR) and FluAB_MLNS 0.3 mg/kg; ^{∘∘∘} p<0.001 vs FluAB_wt, log-rank analysis, Mantel-Cox method.

FIG. 6 shows for Example 5 the circulating levels of the injected antibodies. The individual levels (μg/ml) of circulating FluAB_wt (circles) and FluAB_MLNS (squares) measured in the serum of mice, immediately before (Day 0) and 6 days after infection are shown. Bars represent the mean±SD.

FIG. 7 shows for Example 6 the plate scheme used in the in vitro neutralization assay.

FIGS. 8A-8D show for Example 6 the neutralization activity of FluAB_MLNS and Oseltamivir alone on H1N1 (8A, 8C) and H3N2 (8B, 8D) virus infection.

FIGS. 9A-9B show for Example 6 the combined neutralization activity of FluAB_MLNS and Oseltamivir on H1 (9A) and H3 (9B) virus infection. Data show the inhibited fraction by FluAB_MLNS alone and in combination with heteromolar concentrations of Oseltamivir both in H1N1 (9A) and H3N2 (9B) viral infection of MDCK cells. Data are represented as mean±SD of triplicate values, each replicate obtained in three independent culture plates.

FIGS. 10A-10B show for Example 6 the median effect plots of combined FluAB_MLNS and Oseltamivir. The two compounds were serially diluted at the indicated constant ratios and added to MDCK cells infected with either H1 (10A) and H3 (10B) viral strains. The values obtained from selected combinations at non-constant ratios (NCR) are also shown.

FIGS. 11A-11F show for Example 6 the combination indexes of FluAB_MLNS and Oseltamivir for H1N1 virus infection. Dots represent the actual experimental points at the indicated constant ratios with the cumulated drug-drug concentration denoted aside. The dotted curves show the predicted combination index across the complete effect range.

FIGS. 12A-12E show for Example 6 the combination indexes of FluAB_MLNS and Oseltamivir for H3N2 virus infection. Dots represent the actual experimental points at the indicated constant ratios with the cumulated drug-drug concentration denoted aside. The dotted curves show the predicted combination index across the complete effect range.

FIGS. 13A-13C show for Example 6 isobolograms of FluAB_MLNS-Oseltamivir combinations for H1N1 virus infection. Dots show the IC₅₀, IC₇₅ and IC₉₀ values on different constant ratio FluAB_MLNS-Oseltamivir combinations. For each experimental point, the cumulated concentration is shown.

FIGS. 14A-14C show for Example 6 isobolograms of FluAB_MLNS-Oseltamivir combinations for H3N2 virus infection. Dots show the IC₅₀, IC₇₅ and IC₉₀ values on different constant ratio FluAB_MLNS-Oseltamivir combinations. For each experimental point, the cumulated concentration is shown.

FIGS. 15A-15D show for Example 6 the neutralization activity of FluAB_MLNS and Zanamivir alone on H1N1 (15A, 15C) and H3N2 (15B, 15D) virus infection.

FIGS. 16A-16B show for Example 6 the combined neutralization activity of FluAB_MLNS and Zanamivir on H1 (A) and H3 (B) virus infection. Data show the inhibited fraction by FluAB_MLNS alone and in combination with heteromolar concentrations of Zanamivir both in H1N1 (A) and H3N2 (B) viral infection of MDCK cells. Data are represented as mean±SD of triplicate values, each replicate obtained in three independent culture plates.

FIGS. 17A-17B show for Example 6 the median effect plots of combined FluAB_MLNS and Zanamivir. The two compounds were serially diluted at the indicated constant ratios and added to MDCK cells infected with either H1 (17A) and H3 (17B) viral strains. The values obtained from selected combinations at non-constant ratios (NCR) are also shown.

FIGS. 18A-18D show for Example 6 the combination indexes of FluAB_MLNS and Zanamivir for H1N1 virus infection. Dots represent the actual experimental points at the indicated constant ratios with the cumulated drug-drug concentration denoted aside. The dotted curves show the predicted combination index across the complete effect range.

FIGS. 19A-19D show for Example 6 the combination indexes of FluAB_MLNS and Zanamivir for H3N2 virus infection. Dots represent the actual experimental points at the indicated constant ratios with the cumulated drug-drug concentration denoted aside. The dotted curves show the predicted combination index across the complete effect range.

FIGS. 20A-20C show for Example 6 isobolograms of FluAB_MLNS-Zanamivir combinations for H1N1 virus infection. Dots show the IC₅₀, IC₇₅ and IC₉₀ values on different constant ratio FluAB_MLNS-Zanamivir combinations. For each experimental point, the cumulated concentration is shown.

FIGS. 21A-21C show for Example 6 isobolograms of FluAB_MLNS-Zanamivir combinations for H3N2 virus infection. Dots show the IC₅₀, IC₇₅ and IC₉₀ values on different constant ratio FluAB_MLNS-Zanamivir combinations. For each experimental point, the cumulated concentration is shown.

FIGS. 22A-22D show for Example 6 the neutralization activity of FluAB_MLNS and Baloxavir alone on H1N1 (22A, 22C) and H3N2 (22B, 22D) virus infection.

FIGS. 23A-23B shows for Example 6 the combined neutralization activity of FluAB_MLNS and Baloxavir on H1 (23A) and H3 (23B) virus infection. Data show the inhibited fraction by FluAB_MLNS alone and in combination with heteromolar concentrations of Baloxavir both in H1N1 (23A) and H3N2 (23B) viral infection of MDCK cells. Data are represented as mean±SD of triplicate values, each replicate obtained in three independent culture plates.

FIGS. 24A-24B show for Example 6 the median effect plots of combined FluAB_MLNS and Baloxavir. The two compounds were serially diluted at the indicated constant ratios and added to MDCK cells infected with either H1 (24A) and H3 (24B) viral strains. The values obtained from selected combinations at non-constant ratios (NCR) are also plotted.

FIGS. 25A-25F show for Example 6 the combination indexes of FluAB_MLNS and Baloxavir. Dots represent the actual experimental points at the indicated constant ratios with the cumulated drug-drug concentration denoted aside. The dotted curves show the predicted combination index across the complete effect range.

FIG. 26A-26F show for Example 6 isobolograms of FluAB_MLNS-Baloxavir combinations. Dots show the IC₅₀, IC₇₅ and IC₉₀ values on different constant ratio FluAB_MLNS-Baloxavir combinations. For each experimental point, the cumulated concentration is shown.

FIGS. 27A and 27B show for Example 7 a patient schedule of assessments (“Part A” patients in Example 7) in a clinical study of FluAB_MLNS.

FIGS. 28A-28B show for Example 7 a patient schedule of assessments (“Part B” patients in Example 7) in a clinical study of FluAB_MLNS.

FIGS. 29A-29B show for Example 7 a patient schedule of assessments (“Part C” patients in Example 7) in a clinical study of FluAB_MLNS.

FIG. 30 shows for Example 7 an influenza-like monitoring schedule used in a clinical study of FluAB_MLNS.

FIG. 31 shows for Example 7 pharmacokinetic assessment timepoints used in a clinical study of FluAB_MLNS.

FIG. 32 shows for Example 7 a list of laboratory assessments used in a clinical study of FluAB_MLNS.

FIG. 33 shows populations at high risk of developing influenza-related complications.

FIGS. 34 and 35 show serum concentration of FluAB_MLNS in human subjects administered FluAB_MLNS in accordance with a clinical trial described in Example 7. FIG. 34 shows the antibody serum concentration in patients receiving 300 mg and 1,200 mg initial doses of FluAB_MLNS to 20 and 12 weeks, respectively. FIG. 35 shows projected antibody serum concentrations of FluAB_MLNS up to 6 months after administration of 300 mg and 1,200 mg doses to a subject.

FIG. 36 shows antibody serum concentration of FluAB_MLNS in human subjects receiving FluAB_MLNS in accordance with a clinical trial described in Example 7. Antibody serum concentrations out to 20 weeks post-administration are provided for subjects receiving of 60 mg, 300 mg, 1,200 mg, and 1,800 mg initial doses of FluAB_MLNS.

FIG. 37 provides a table showing various FluAB_MLNS PK parameters observed in the clinical study detailed in Example 7.

FIGS. 38A and 38B show binding of human FcRn in solution to immobilized FluAB_MLNS or FluAB_wt as measured by Octet at pH=6.0 (A) or pH=7.4 (B). The time point 0 seconds represents switch from base line buffer to buffer containing human FcRn. Time point 420 seconds (dotted vertical line) represents switch to blank buffer at the corresponding pH. Association and dissociation profiles were measured in real time using an Octet RED96 (ForteBio).

FIGS. 39A and 39B show levels of ADA response measured by ELISA to detect mouse anti-drug IgG (A; bars represent the mean±SD of treatment group); and correlation analysis (B) between the levels of circulating human IgG measured 14 days after i.v. injection (X axis) and the signal of the ADA present at the same time point (Y axis). The non-parametric Spearman's correlation coefficient is shown for the significant values.

FIG. 40 shows levels of ADA response after subcutaneous (s.c.) injection of either FluAB_MLNS or FluAB_wt. Data are represented as values of the ADA signal (OD 450 nm) detected in each individual serum obtained three weeks after the s.c. injection (n=5/group), pre-diluted in PBS 1:25 and then further serially diluted 5-fold.

### EXAMPLES

In the following, particular examples illustrating embodiments and aspects of the disclosure are presented. However, the present disclosure shall not to be limited in scope by the specific embodiments described herein. The following preparations and examples are given to enable those skilled in the art to more clearly understand and to practice the present disclosure. The present disclosure, however, is not limited in scope by the exemplified embodiments. Indeed, various modifications of the disclosure in addition to those described herein will become readily apparent to those skilled in the art from the foregoing description, accompanying figures and the examples below. All such modifications fall within the appended claims.

### Example 1

### Safety and Tolerability of an Antibody According to the Present Disclosure in Cynomolgus Macaques

An antibody according to the present disclosure, which comprises (i) the CDR sequences as set forth in SEQ ID NOs 1-6 and (ii) the two mutations M428L and N434S in the heavy chain constant regions, was designed and produced. More specifically, the antibody comprises (i) the heavy chain variable region (VH) sequence as set forth in SEQ ID NO: 7 and the light chain variable region (VL) sequence as set forth in SEQ ID NO: 8; and (ii) the two mutations M428L and N434S in the heavy chain constant regions. Even more specifically, the antibody comprises a heavy chain having an amino acid sequence as set forth in SEQ ID NO: 9 and a light chain having an amino acid sequence as set forth in SEQ ID NO: 10. This antibody is referred to herein as “FluAB_MLNS”.

For comparison, antibody “FluAB_wt” was used, which differs from antibody “FluAB_MLNS” only in that it does not contain the mutations M428L and N434S in the heavy chain constant regions. Accordingly, comparative antibody “FluAB_wt” comprises a heavy chain having an amino acid sequence as set forth in SEQ ID NO: 11 and a light chain having an amino acid sequence as set forth in SEQ ID NO: 10.

A single intravenous infusion of 5 mg/kg of either FluAB_MLNS or FluAB_wt in a 2.5 ml/kg volume was given in a 60-minutes intravenous infusion to three female cynomolgus macaques (Macaca fascicularis) per test group. Blood or urine for clinical chemistry and hematological analyses were collected pre-dose and on days 7 and 21 post-dose.

Following dosing of either FluAB_MLNS or FluAB_wt at 5 mg/kg in a 60-minutes intravenous infusion, the female cynomolgus macaques were closely monitored for health and weight and regularly sampled for blood and urine. No adverse events — other than bruising 24 h and erythroderma 3 days post-dose at the inoculation site in some of the animals — were observed following intravenous inoculation of the antibodies. All animals were generally healthy, showed normal food consumption, and had overall positive weight gain throughout the study. Clinical chemistry, hematology, and urinalysis parameters were normal at 7- or 21-days post dosing, compared to pre-dosing samples.

In summary, a single intravenous infusion of either FluAB_MLNS or FluAB_wt into cynomolgus macaques did not induce adverse events and was generally well tolerated.

### Example 2

### Determination of Plasma Concentration and Pharmacokinetics

These experiments aimed to determine the concentration, establish half-life, and compare the pharmacokinetics of the antibody according to the present disclosure FluAB_MLNS in comparison to comparative antibody FluAB_wt in the plasma following a single intravenous injection.

Before dosing, the animals were tested to be negative for influenza-specific antibodies using dot immunobinding assay. Seropositive animals were excluded from the study as pre-existing immunity may interfere with this test. In addition, animals developing anti-drug antibody (ADA) response were excluded.

A single intravenous infusion of 5 mg/kg of either FluAB_MLNS or FluAB_wt in a 2.5 ml/kg volume was given in a 60-minutes intravenous infusion to three female macaques per test group. Blood was collected in tubes containing K₂EDTA pre-dose and processed to plasma for pharmacokinetic testing after approximately 1, 6, 24, 96, 168, 504, 840, and 1344 hours (h) post-dose.

Plasma concentration of the antibodies was determined in vitro using an ELISA assay. Briefly, IAV-HA antigen (Influenza A virus H1N1 A/California/07/2009 Hemagglutinin Protein Antigen (with His Tag); Sino Biologicals) was diluted to 2 μg/ml in PBS and 25 μl were added to the wells of a 96-well flat bottom ½-area ELISA plate for coating over night at 4° C. After coating, the plates were washed twice with 0.5× PBS supplemented with 0.05% Tween20 (wash solution) using an automated ELISA washer. Then, plates were blocked with 100 μl/well of PBS supplemented with 1% BSA (blocking solution) for 1 h at room temperature (RT) and then washed twice. Plasma samples were centrifuged at 10,000 g for 10 min at 4° C. and then diluted (1:10 and then 1:30) for a final 1:300 dilution in blocking solution in 96-well cell culture plates.

The minimum dilution (1:300) of the macaque plasma used for quantification was tested and set to ensure that the matrix effect was negligible. Samples were then diluted 1:2 stepwise in triplicates for a total of 12 dilutions. Standards for each antibody to be tested were prepared similarly via diluting the antibodies 1:300 to 1 μg/ml in a pool of pre-inoculation plasma from all test animals, mimicking the matrix of the test samples. Standards were then diluted 1:3 stepwise in blocking solution in triplicates for a total of 12 dilutions. Twenty-five μl of the prepared samples or standards were added to hemagglutinin (HA)-coated wells and incubated for 1 h at RT. After four washes, 25 μl of goat anti human-IgG HRP conjugate (AffiniPure F(ab′)₂ Fragment, Fcγ Fragment-Specific; Jackson ImmunoResearch) diluted in blocking solution 1:5,000 (final concentration 0.16 μg/ml) were added per well for detection and incubated at RT for 1 h. After four washes, plates were developed by adding 40 μl per well of SureBlue TMB Substrate (Bioconcept). After ˜7-20 min incubation at RT, when the color reaction reached a plateau (max OD ˜3.8), 40 μl of 1% HCl were added per well to stop the reaction and absorbance was measured at 450 nm using a spectrophotometer.

To determine the concentration of the antibodies in cynomolgus plasma, OD values from ELISA data were plotted vs. concentration in the Gen5 software (BioTek). A non-linear curve fit was applied using a variable slope model, four parameters and the equation: Y=(A−D)/(1+(X/C)\hat{ }B)+D). The OD values of the sample dilutions that fell within the predictable assay range of the standard curve—as determined in setup experiment by quality control samples in the upper, medium or lower range of the curve—were interpolated to quantify the samples. Plasma concentration of the antibodies were then determined considering the final dilution of the sample. If more than one value of the sample dilutions fell within the linear range of the standard curve, an average of these values was used. Pharmacokinetics (PK) data were analyzed by using WINNONLIN NONCOMPARTMENTAL ANALYSIS PROGRAM (8.1.0.3530 Core Version, Phoenix software, Certara) with the following settings: Model: Plasma Data, Constant Infusion Administration; Number of non-missing observations: 8; Steady state interval Tau: 1.00; Dose time: 0.00; Dose amount: 5.00 mg/kg; Length of Infusion: 0.04 days; Calculation method: Linear Trapezoidal with Linear Interpolation; Weighting for lambda_z calculations: Uniform weighting; Lambda_z method: Find best fit for lambda_z, Log regression. Graphing and statistical analyses (linear regression or outlier analysis) were performed using Prism 7.0 software (GraphPad, La Jolla, Calif., USA). Outlier analysis was performed using the ROUT method (Q=1%), with the potential to find any number of outliers in either direction.

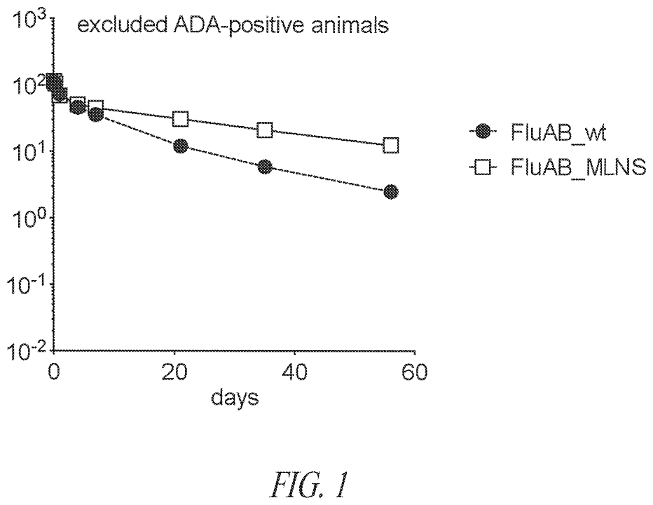

Results are shown in FIG. 1. Analysis of cynomolgus plasma samples drawn up to 56 days post-inoculation demonstrated that the antibody according to the present disclosure FluAB_MLNS had an extended in-vivo half-life compared to comparative antibody FluAB_wt (FIG. 1). Using noncompartmental analysis with WinNonLin, the T_{1/2} was estimated as 19.5 days for FluAB_MLNS, while T_{1/2} was estimated as 11.6 days for the comparative antibody FluAB_wt. The lower limit of quantification was 300 ng/ml.

In summary, FluAB_MLNS had an extended in-vivo half-live compared to comparative antibody FluAB_wt at least up to day 56 post-inoculation.

### Example 3

### Long-Term Stability In Vivo

To test in-vivo stability and functionality of the antigen binding of FluAB_MLNS over time, the pharmacokinetics measurement (as described in Example 2) of the group receiving FluAB_MLNS was extended to days 86 and 113 post-inoculation. On days 1, 21, 56, 86, 113 post-inoculation, functional FluAB_MLNS was quantified using the hemagglutinin (HA) binding ELISA as described in Example 2.

Further, total human antibodies in macaque plasma was quantified using a specific anti-CH2 ELISA, using a capture mAb that specifically binds the CH2 region of human but not of monkey Abs. To measure total human IgG and thus quantify total inoculated human antibodies in cynomolgus plasma, an ELISA capturing with mouse anti-CH2 domain-specific to human IgG (clone R10Z8E9; Thermo Scientific) was used. It was verified that this mAb does not cross-react with monkey IgG. For coating of 96-well flat bottom ½-area ELISA plates, mouse anti-human IgG CH2 was added in PBS at 0.5 μg/ml and incubated over night at 4° C. Then, plates were washed and 100 μl/well blocking solution with 5% BSA was added for 1 h at RT. Standards of FluAB_MLNS were prepared via diluting FluAB_MLNS to 1 ng/ml in blocking solution. Standards were then diluted 1:1.5 stepwise in blocking solution in duplicates for a total of 12 dilutions. Cynomolgus plasma samples were centrifuged at10,000 g for 10 min at 4° C. and step-wise diluted to a final 1:1,000, 1:5,000 or 1:15,000 in blocking solution. After washing the plate, 25 μl of samples or standard were added to the ELISA plate and incubated for 1 h at RT. After three washes, 25 μl of goat anti human-IgG HRP (AffiniPure F(ab′)₂ Fragment, Fcγ Fragment-Specific; Jackson ImmunoResearch) at 0.04 μg/ml were added in blocking solution with 1% BSA for detection and incubated at RT for 45 min. After three washes, plates were developed by adding 40 μl per well of SureBlue TMB Substrate (Bioconcept). After 20 min incubation at RT, 40 μl of 1% HCl were added to stop the reaction, and absorbance was measured at 450 nm.

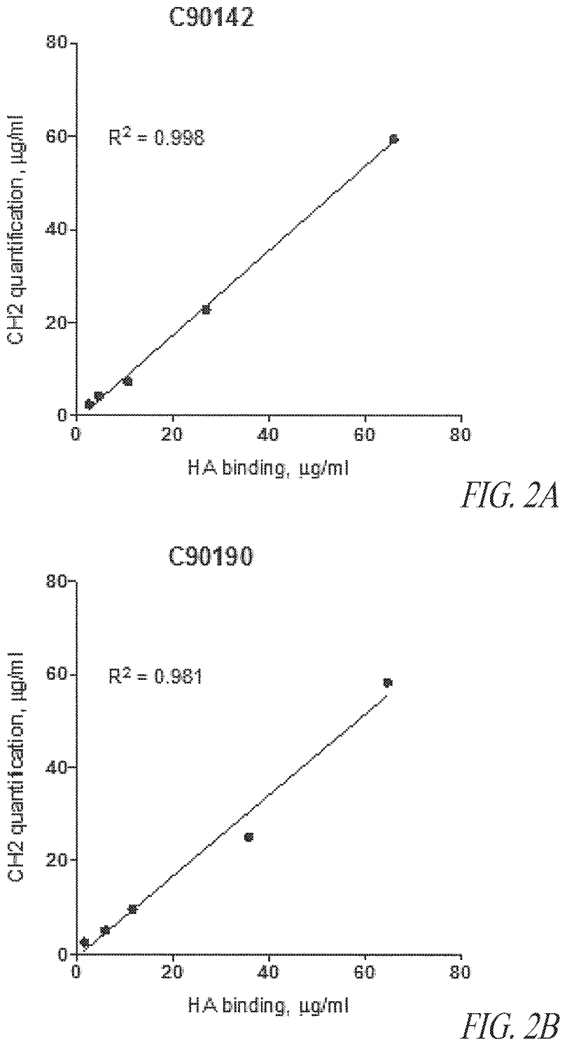

Results are shown in FIGS. 2A-2B. Both quantifications resulted in similar human antibody concentrations in cynomolgus plasma (FIGS. 2A-2B). Additional analysis via linear regression demonstrated that the relation between quantification via HA binding and total anti-CH2 quantification followed a linear patter for all selected time points. Consequently, the total amount of FluAB_MLNS present in plasma was functional in binding to the hemagglutinin (HA) stem region of influenza A virus (IAV), also after 86 and 113 days in vivo.

In summary, FluAB_MLNS demonstrated functional antigen binding and thus good long-term stability in vivo up to day 113 post-inoculation during study extension.

### Example 4

### Antibody Concentration in Nasal Swabs and Biodistribution

To determine biodistribution of FluAB_MLNS and of the comparative antibody FluAB_wt between the nasal mucus relative to plasma, the concentration of the antibody was determined in nasal swabs. To this end, Nasal swabs of the macaques described in Example 2 were collected 24, 504, and 1344 hours after administration of FluAB_MLNS or of the comparative antibody FluAB_wt. Concentrations of antibodies FluAB_MLNS and FluAB_wt in nasal swabs were determined essentially as described in Example 2 for determination in plasma with the following minor adaptations: (a) ELISA plates were blocked 2 h at RT; (b) Nasal swab samples were diluted starting at 1:2 with 1% BSA in PBS and then serially diluted step-wise 1:2 for a total of 8 dilution points; (c) nasal swab medium (RT MINI Viral Transport Medium; Copan) was used as assay matrix control.

To eliminate differences during the swabbing procedure or in the amount of nasal secretions present in each animal and at different time points (days 1, 21, and 56), results from nasal swabs were normalized to urea content. Urea freely diffuses between blood, being present in similar amounts across these plasma or swab samples (Lim et al., 2017, Antimicrob Agents Chemother 61(8):e00279-17). To this end, Urea Nitrogen (BUN) was measured quantitatively using the “Urea Nitrogen (BUN) Colorimetric Detection Kit” (Invitrogen), following the manufacture's procedure. In brief, samples were diluted 1:3 in PBS and mixed with the kit reagents A and B and incubated at room temperature for 30 minutes. The colored product of the redox reaction was read at 450 nm using a 96-well microplate reader. Quantification was performed via comparing samples to BUN standards, which were provided with the kit and treated equivalently.

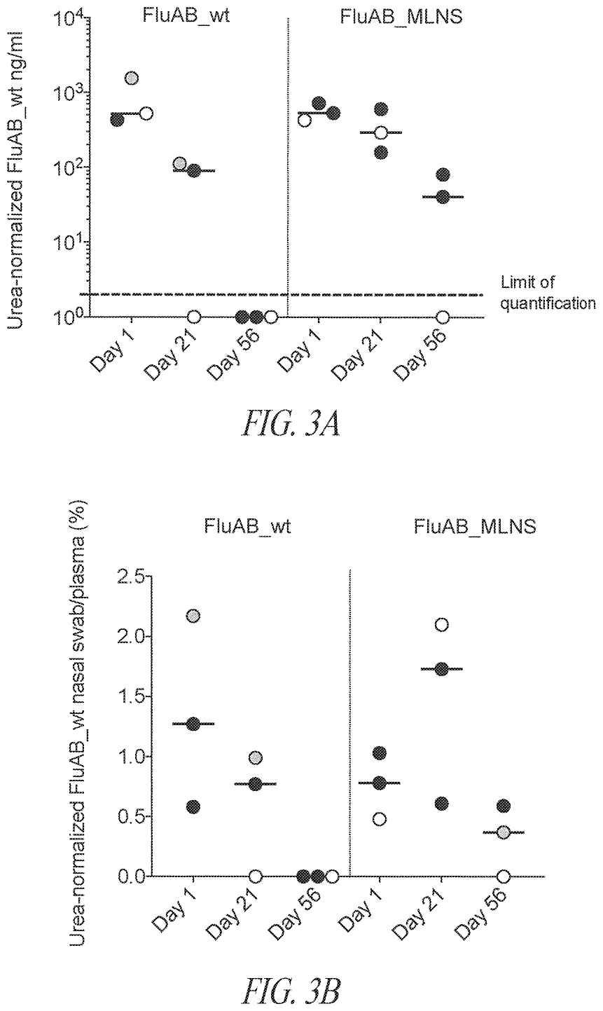

Results are shown in FIGS. 3A-3B. Amounts of normalized antibodies in nasal swabs decreased over time (FIG. 3A). Determining biodistribution via comparing nasal to plasma concentrations revealed no differences between FluAB_MLNS and the comparative antibody FluAB_wt (FIG. 3B), suggesting that the MLNS-Fc mutation, while prolonging the half-life of FluAB_MLNS in plasma, did not enhance bio-distribution of the antibody into the nasal mucus.

In summary, nasal swab samples did not reveal any significant differences in biodistribution between the nasal mucus and plasma amongst the mAb variants.

### Example 5

### Prophylactic Activity of Antibody FluAB_MLNS in PR8-Infected Tg32 Mice

Next, the prophylactic activity of FluAB_MLNS compared to antibody FluAB_wt was determined in a H1N1 murine model of lethal influenza A infection.

To evaluate the prophylactic efficacy, 9- to 14-week-old FcRn−/−hFcRn line 32 Tg mice (C57B6 background) were intravenously (i.v.)-injected (via the tail vein) with 5 ml/kg of a solution containing FluAB_MLNS or the comparator antibody FluAB_wt at doses ranging from 0.3 to 1 mg/kg. Twenty-four hours after the i.v. injection, mice were bled from the tail vein to determine the serum antibody levels before infection. Bleedings were also repeated on day 6 and 13 post infection (p.i.). Both antibody-injected and untreated mice were anesthetized (isoflurane, 4% in O₂, 0.3 L/min) and challenged intranasally (i.n.) by slow instillation in both nostrils of 50 μl (25 μl/each) of PBS containing 5 mouse lethal dose fifty percent (5 MLD₅₀, equivalent to 1200 TCID₅₀/mouse) of influenza virus A (H1N1, A/Puerto Rico/8/34, as described in Cottey, R., Rowe, C. A., and Bender, B. S. (2001). Influenza virus. Curr Protoc Immunol Chapter 19, Unit19.11-19.11.32). Each mouse was held upright with its head tilted slightly back for about 1 minute to reduce the likelihood of inoculum dripping from the nares. After the procedure and upon righting reflex occurrence, animals were returned to the cage. The mice were monitored daily for weight loss and disease symptoms until day 14 p.i. and euthanized if they lost more than 20% of their initial body weight (whereby 0% is set on the day of infection) or reached morbidity score of 4. Table 1 details the applied morbidity score:

TABLE 1
Morbidity Score of PR8-infected mice

| Morbidity Score | Clinical signs |
|---|---|
| 1 | Healthy |
| 2 | Consistently ruffled fur on the neck |
| 3 | Piloerection, possible deeper breathing, less alert |
| 4 | Labored breathing, tremors and lethargy |
| 5 | Abnormal gait, reduced mobility, emaciation, |
| | tail-ears cyanosis |
| 6 | Death |

All the animals were eventually sacrificed to collect serum and lungs.

Serum Preparation:

Approximately 0.05 ml of blood were collected into gel-containing tubes and let stay for 30 min at RT. Tubes were spun for 5 min at 5500 rpm (3200×g), serum was transferred to new tubes and stored at −20° C. until use.

Two independent experiments were carried out, according to the following designs:

TABLE 2
Study Design Experiment 1:

| Group | N of animals | IV Treatment | mAb Dose | |
|---|---|---|---|---|
| 1 | 4 | — | — | |
| 2 | 8 | FluAB_wt | 1 | mg/kg |
| 3 | 4 | FluAB_wt | 0.3 | mg/kg |
| 4 | 8 | FluAB_MLNS | 1 | mg/kg |
| 5 | 4 | FluAB_MLNS | 0.3 | mg/kg |

TABLE 3
Study Design Experiment 2:

| Group | N of animals | IV Treatment | mAb Dose |
|---|---|---|---|
| 1 | 9 | — | — |
| 2 | 10 | FluAB_wt | 0.3 mg/kg |
| 3 | 6 | FluAB_MLNS | 0.3 mg/kg |

ELISA Quantification of Circulating mAb:

Sera were assessed for the levels of circulating antibodies on day 0 and 6. Briefly, half-area ELISA plates were coated over night at 4° C. with recombinant hemagglutinin (HA) from H1N1 strain A/California/07/09 (2 μg/ml, in PBS, 25 μl/well). Following blocking (PBS/1% BSA, 100 μl/well, 1 hr RT) and 2 washes (220 μl/well) with ELISA washing solution (PBST), both dilutions of the sera (initial dilution 1:150 for 1 mg/kg, 1:50 for 0.3 mg/kg) and the antibody standards (FluAB_MLNS and FluAB_wt, 0.1 μg/ml) were added (25 μl/well) in duplicate and serially diluted (1:2 by 10 points for serum dilutions, 1:3 by 8 points for antibody standards). After 1.5 hr RT incubation, plates were washed 4 times with PBST and further incubated 1.5 hr at RT with the HRP-labeled anti-human secondary antibody (0.16 μg/ml, 25 μl/well). After 4 washes with PBST, plates were dispensed with substrate solution (25 μl/well), developed for 14 min and blocked with 1% HCl (v/v, 25 μl/well). Plates were finally read at 450 nm with a spectrophotometer for signal quantification. Concentration values were calculated by using a non-linear regression model (variable slope model, four parameters, GraphPad Prism) of log (agonist) versus response.

Data Analysis:

Data were plotted and analyzed using GraphPad Prism software version 8.0 for Macintosh, GraphPad Software, La Jolla Calif. USA, www.graphpad.com. Continuous variables were assessed for statistically significant difference (p<0.05, 95% confidence interval) by using ordinary 2-way ANOVA corrected with Bonferroni multiple comparison test. Survival data were compared by using log-rank analysis with Mantel-Cox method (p<0.05 considered statistically significant). The data from the two independent experiments described above were pooled.

Results:

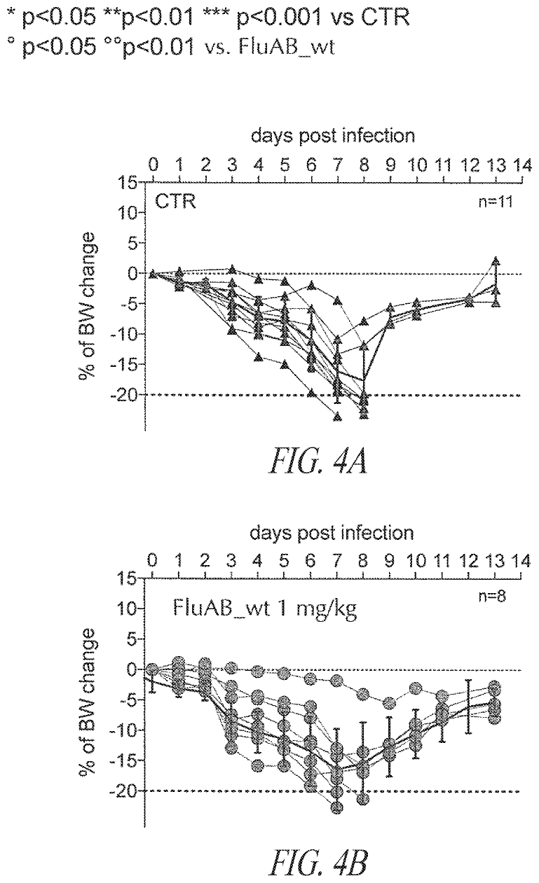

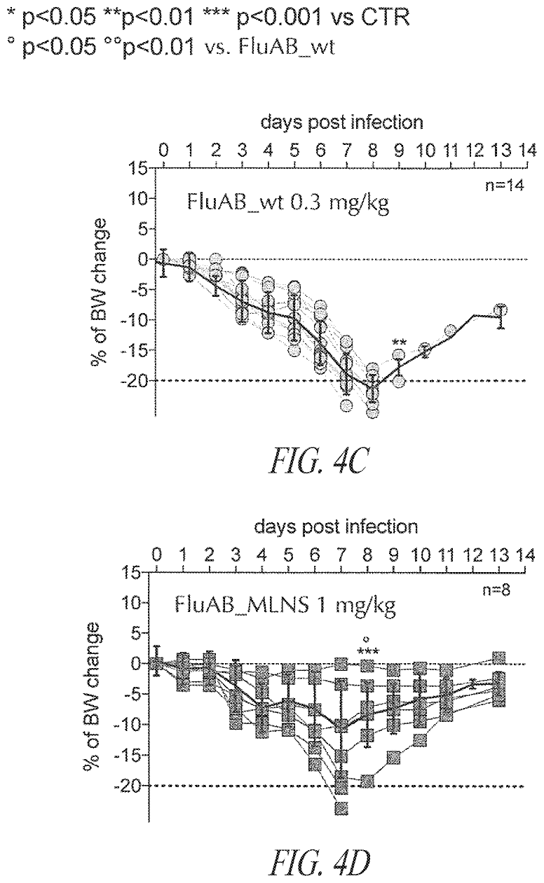

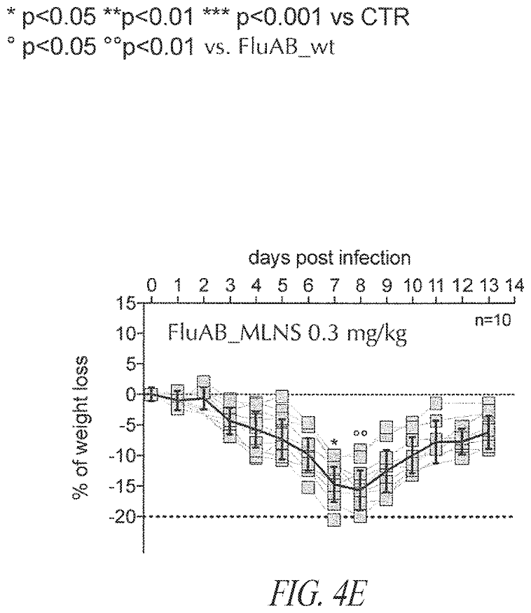

Prophylactic activity was tested upon i.v. administration of FluAB_MLNS and FluAB_MLNS (1 and 0.3 mg/kg) in Tg32 mice one day prior to H1N1 PR8 virus challenge via intranasal infection. Results are shown in FIGS. 4A-6.

As depicted in FIGS. 4A-4E, mice treated with either 1 mg/kg (4D) or 0.3 mg/kg (4E) of FluAB_MLNS showed lower body weight loss, in comparison with both untreated (4A) and FluAB_wt-injected (4B and 4C) mice.

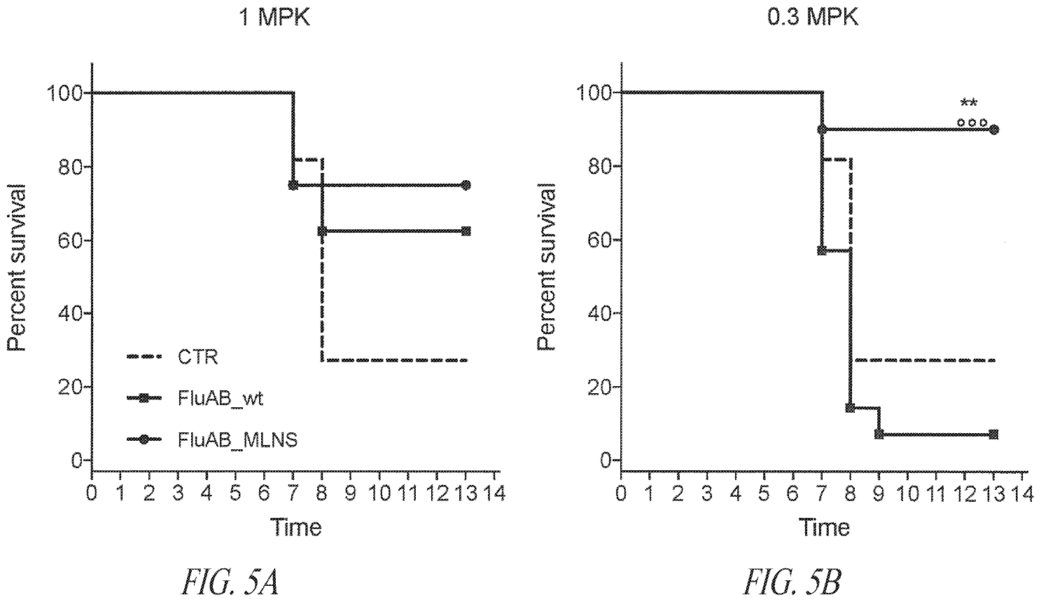

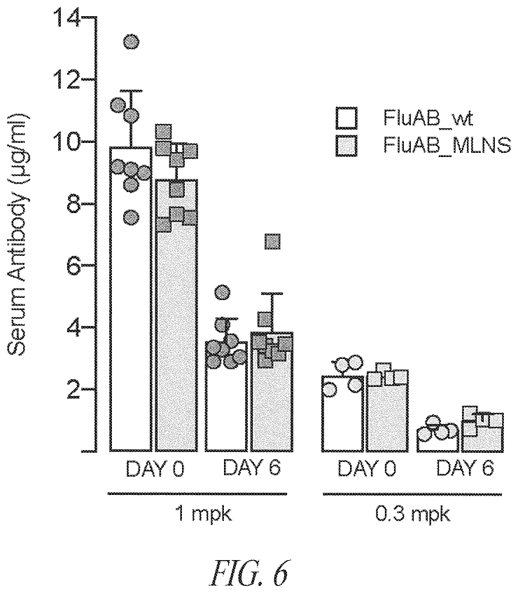

The better protective activity of FluAB_MLNS as compared to FluAB_wt was confirmed in the survival analysis shown in FIGS. 5A and 5B.

The differences in the efficacy between FluAB_MLNS and FluAB_wt did not correlate with different levels of circulating antibodies in the serum, as measured 1 and 7 days after i.v. administration of the antibodies (FIG. 6). Of note, no detectable levels of circulating antibodies were measured 14 days after injection (not shown).

In summary, FluAB_MLNS demonstrated, in Tg32 mice, a better protective capacity against H1N1 PR8 intranasal virus challenge over the comparative antibody FluAB_wt. The efficacy was independent of the circulating antibody levels. These data suggest that the enhanced interaction of FluAB_MLNS with hFcRn expressed by Tg32 mice also mediates in vivo effects unrelated to the extended antibody half-life, such as increased efficacy regarding the protective activity.

### Example 6

### Combination of Antibody FluAB_MLNS with Various Antivirals

Drug combinations offer the clear opportunity to enhance the potency while reducing the probability to select resistances. Moreover, a putative additive or synergic effect may end up to a dose-sparing approach. Influenza medications currently approved by FDA include the neuraminidase inhibitors oseltamivir and zanamivir as well as the recently approved baloxavir marboxil, which belongs to the endonuclease inhibitors class.

To evaluate the combined activity of FluAB_MLNS with the antivirals oseltamivir, zanamivir or baloxavir marboxil on both H1N1 and H3N2 representative viral strains, in vitro neutralization was performed to evaluate the resulting inhibitory effect. The analysis of the combined effects was carried out by using the median-effect plot and the calculation of the combination index (CI).

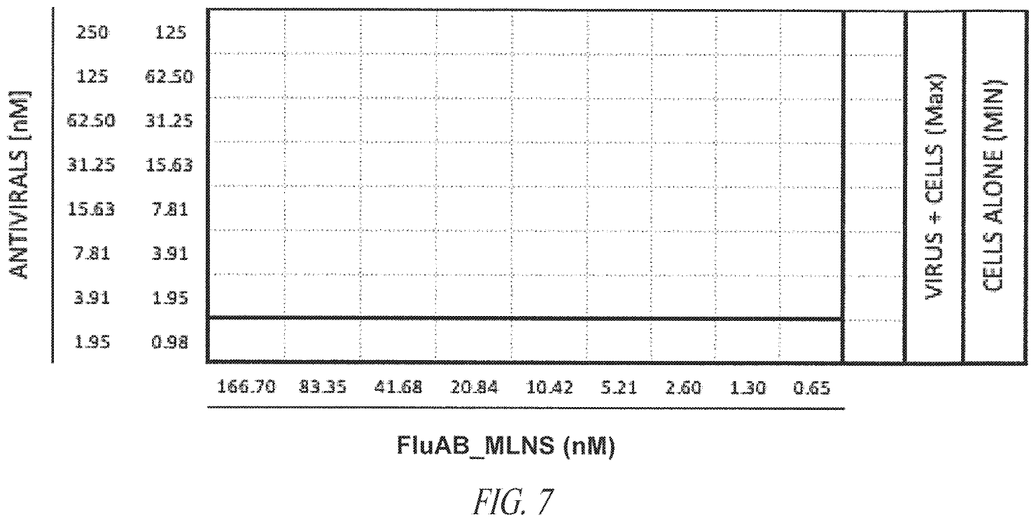

Briefly, MDCK (Madin-Darby canine kidney) cells were seeded at 30,000 cells/well into 96-well plates (flat bottom, black). Cells were cultured at 37° C. 5% CO₂ overnight. Twenty-four hours later, 4× antibody and antiviral (oseltamivir, zanamivir or baloxavir marboxil) dilutions in 60 μl infection medium (MEM (Sigma Aldrich, cat. n. M0644)+Glutamax (Invitrogen, 41090-028)+1 μg/ml TPCK-treated Trypsin (Worthington Biochemical #LS003750)+10 μg/ml Kanamycin) were prepared by using crisscross 1:2 serial dilutions of FluAB_MLNS (starting from 166.7 nM final, 9 horizontal points) and different antivirals (oseltamivir, zanamivir or baloxavir marboxil), starting from 125 (250 for zanamivir) nM by 7 vertical points), according to the plate scheme shown in FIG. 7.

For each combination, three independent plates were prepared, in order to have triplicates of each drug-drug combination ratio. The single compound titration (namely, FluAB_MLNS, 9 points and each antiviral, 8 points) was included in each plate. Virus solution was prepared at concentrations of 120× the TCID50 in 60 μl, further diluted either 1:1 in MEM or mixed 1:1 with FluAB_MLNS dilutions and incubated 1 h at 33° C. Cells were washed 2 times using 200 μl/well MEM without supplements, followed by the addition of either 100 μl of virus alone or 100 μl of FluAB_MLNS/virus mix (100× TCID50/well) and incubated 4 hours at 33° C. 5% CO2. After the addition of 100 μl/well of infection medium, cells were further incubated for 72 hours at 33° C. 5% CO2. On day 3 after infection, 20 μM MuNANA (4-MUNANA (2_-(4-Methylumbelliferyl)-□-D-N-acetylneuraminic acid sodium salt hydrate (Sigma-Aldrich) #69587) solution was prepared in MuNANA buffer (MES 32.5 mM/CaCl₂ 4 mM, pH 6.5) and 50 μl/well was dispensed into black 96-well plates. Fifty μk of either neutralization or virus-alone titration supernatant were transferred to the plates and incubated 60 min at 37° C. The reaction was then stopped with 100 μl/well 0.2 M glycine/50% EtOH, pH 10.7. Fluorescence was quantified at 460 nm with a fluorimeter (Bio-Tek).

The fraction of virus neutralization was calculated according to the formula:

$$1 - ( \frac{f x - f min}{f max} ) ,$$

wherein fx=sample fluorescence signal (cells+virus+FluAB_MLNS+antiviral); fmin=minimal fluorescence signal (cells alone, no virus); fmax=maximal fluorescence signal (cells+virus only).

The neutralized fraction data were used to compute the quantitative analysis of dose-effect relationships for drug-drug combinations according to the Chou and Talalay method (Chou T C, Talalay P: Quantitative analysis of dose-effect relationships: the combined effects of multiple drugs or enzyme inhibitors. Adv. Enzyme Regul. 1984, 22:27-55). The combination Index, the fraction affected (Fa), and isobolograms were obtained by using the CompuSyn software (ComboSyn Inc., Paramus, N.J., USA) (Chou T-C: Theoretical basis, experimental design, and computerized simulation of synergism and antagonism in drug combination studies. Pharmacological Reviews 2006, 58:621-681).

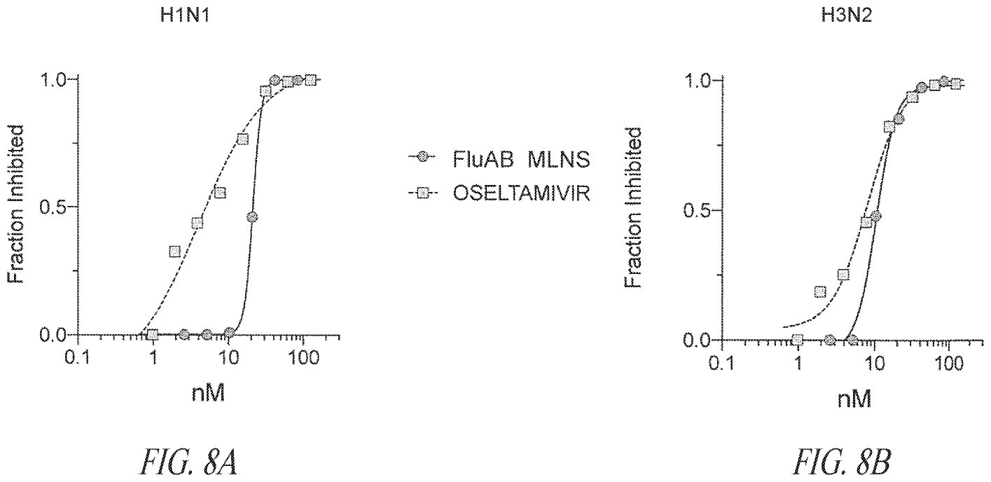

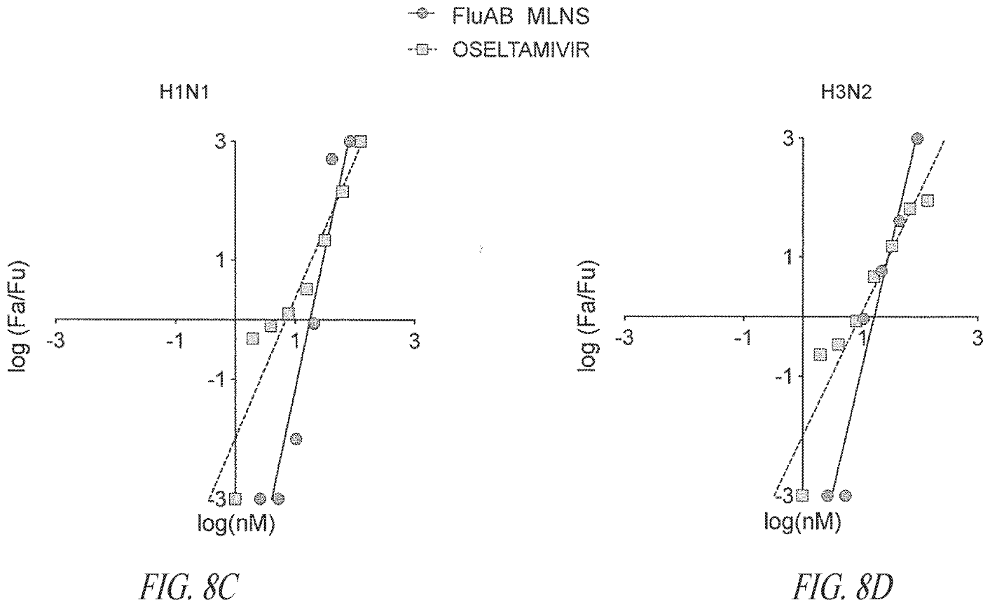

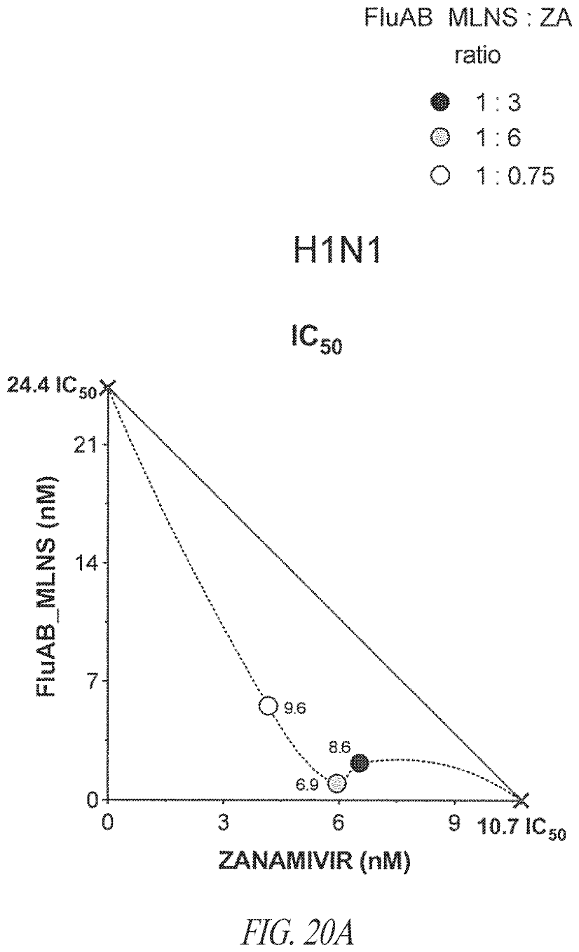

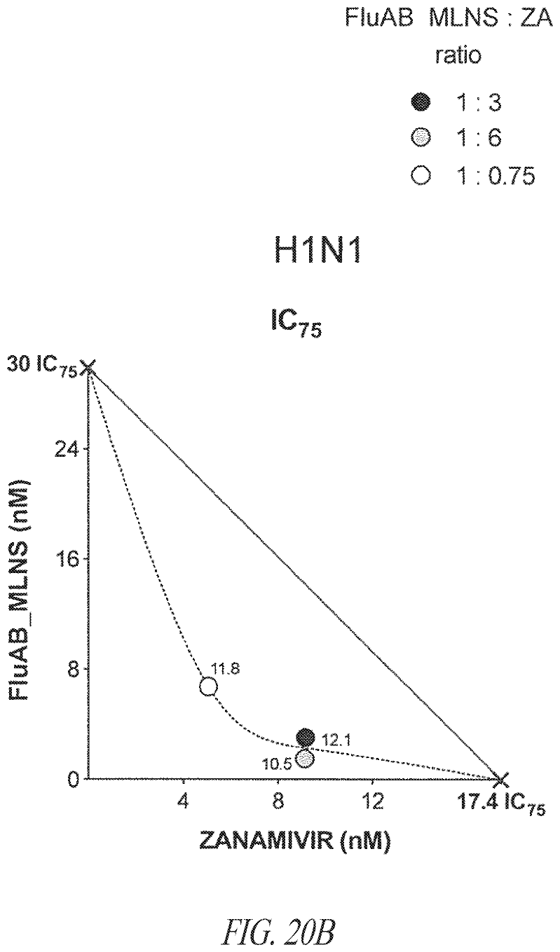

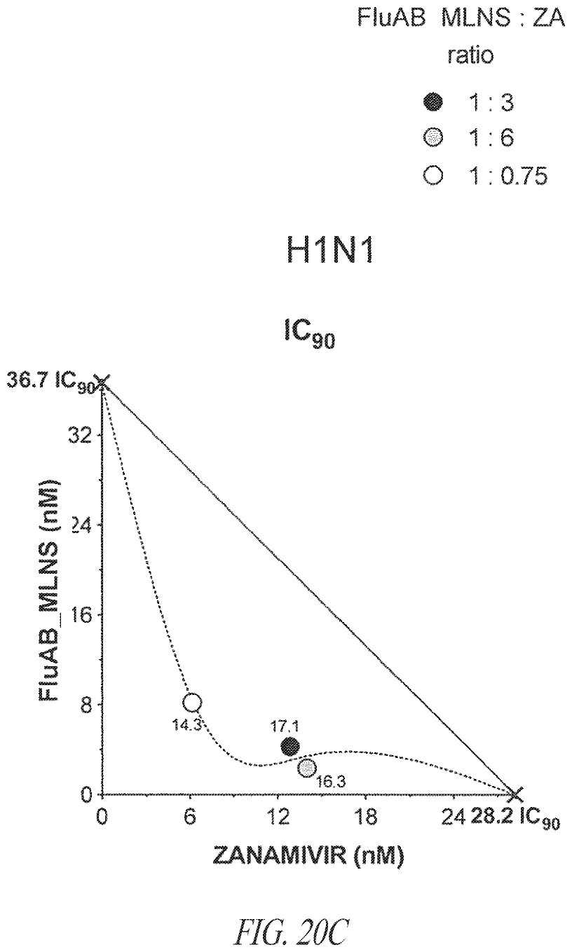

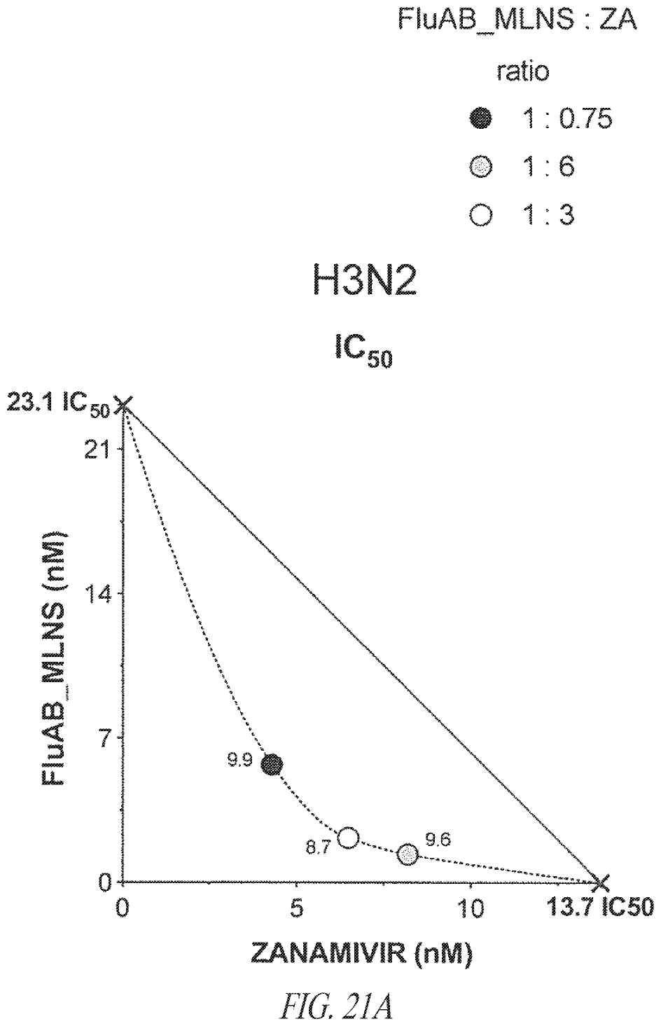

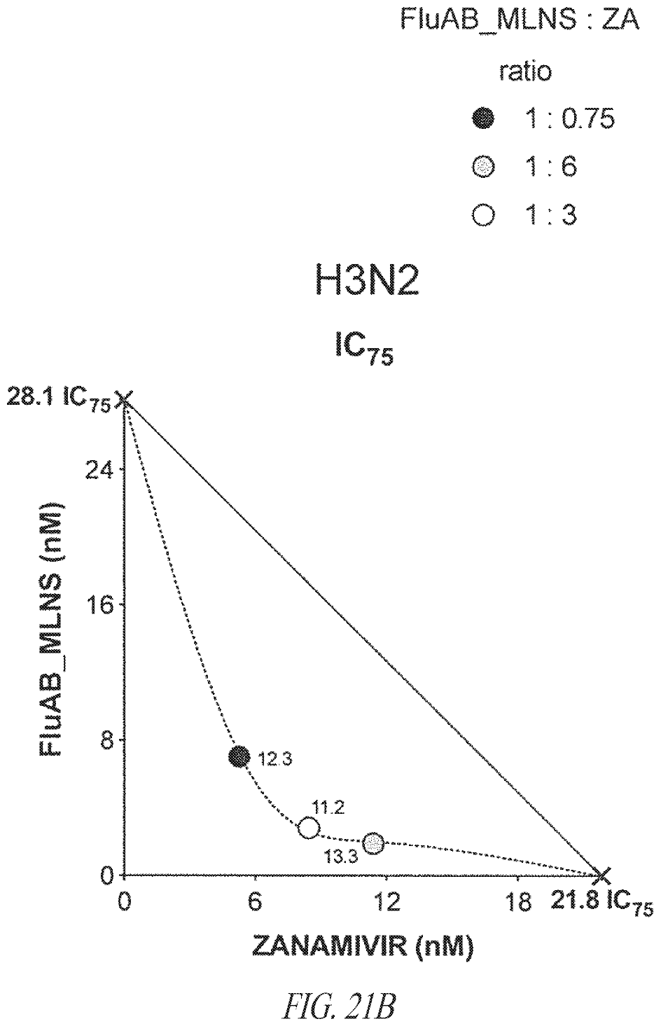

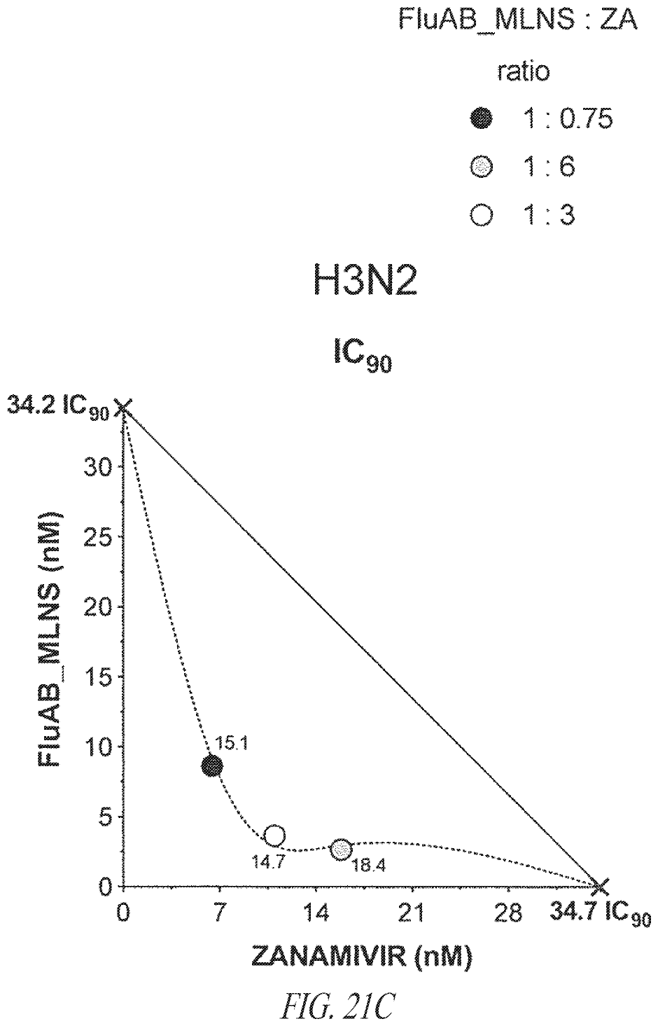

Results are shown in FIGS. 8A-26F and described below.

Combination of FluAB_MLNS and Oseltamivir

The relative efficacy of FluAB_MLNS and oseltamivir to neutralize influenza A viruses was compared in vitro on two viral serotype representatives for both H3N2 and H1N1 strains. As shown in FIGS. 8A-8D, both compounds, tested separately, were dose-dependently capable to fully inhibit cell infection, when independently exposed together with H3N3 and H1N1 virus (FIGS. 8A, 8B). The IC₅₀ values, as calculated from the median-effect plot (FIGS. 8C, 8D) after data log linearization (as described in (Chou TC, Talalay P: Quantitative analysis of dose-effect relationships: the combined effects of multiple drugs or enzyme inhibitors. Adv. Enzyme Regul. 1984, 22:27-55) are indeed in the nanomolar range for both FluAB_MLNS (17.9 and 15.6 nM for H3 and H1 strains respectively) and oseltamivir (7 and 9.1 nM for H3 and H1 strains respectively). Overall, no substantial differences were measured in terms of inhibitory response by FluAB_MLNS between H3 and H1 virus infection, while H1N1 virus resulted marginally more sensitive to the inhibitory effect of oseltamivir.

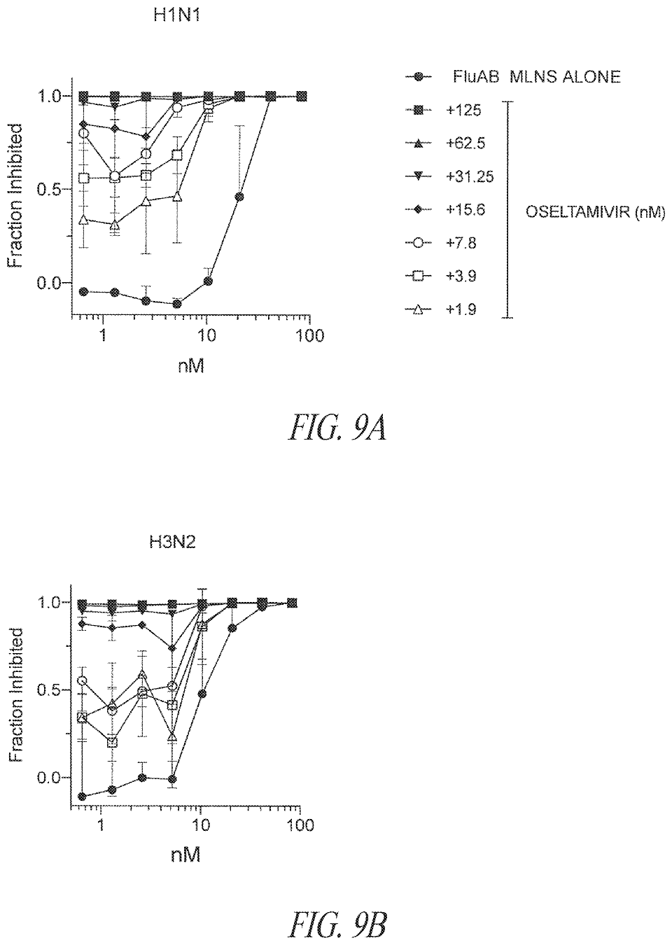

To test the effect of a combination of FluAB_MLNS and oseltamivir in neutralizing the infection of MDCK cells with H3 and H1 virus, both compounds were serially diluted at different ratios as described above, and assessed for the enzymatic activity of neuraminidase (NA; as a read out of the viral content in the culture) in the presence of the different drug concentrations and compared to the single drug effects. The neutralization effect measured with FluAB_MLNS is greatly enhanced by the concomitant presence of heteromolar concentrations of the second compound, thus suggesting a synergistic effect rather than an addictive one, both on H3 and H1 virus infection (FIGS. 9A-9B). A slightly different susceptibility of H1 and H3 viruses to the inhibitory action of oseltamivir was detected.

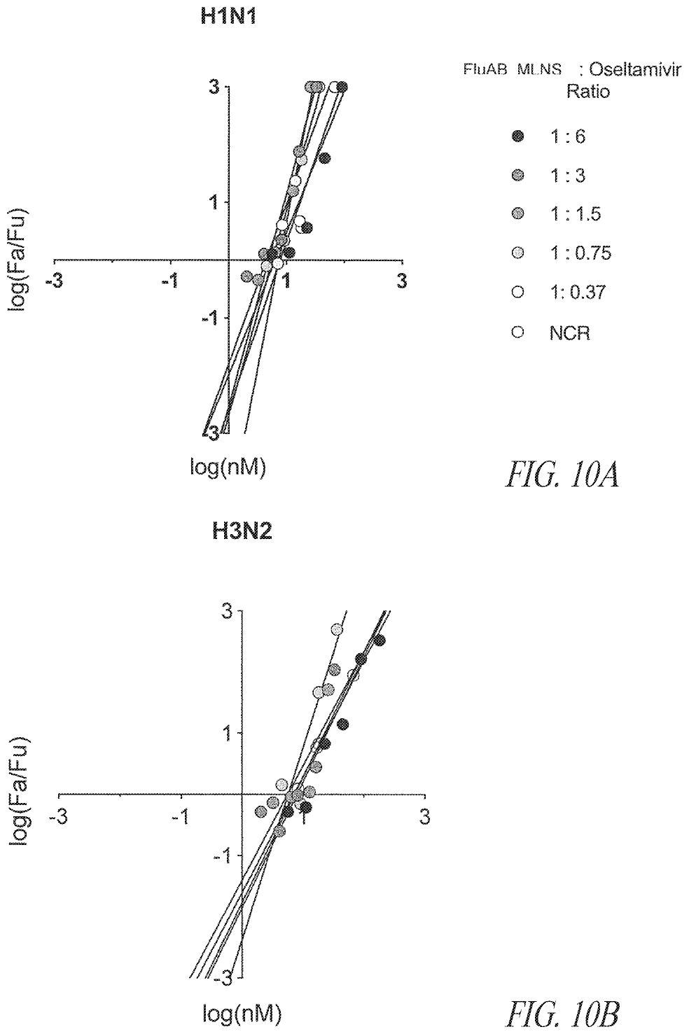

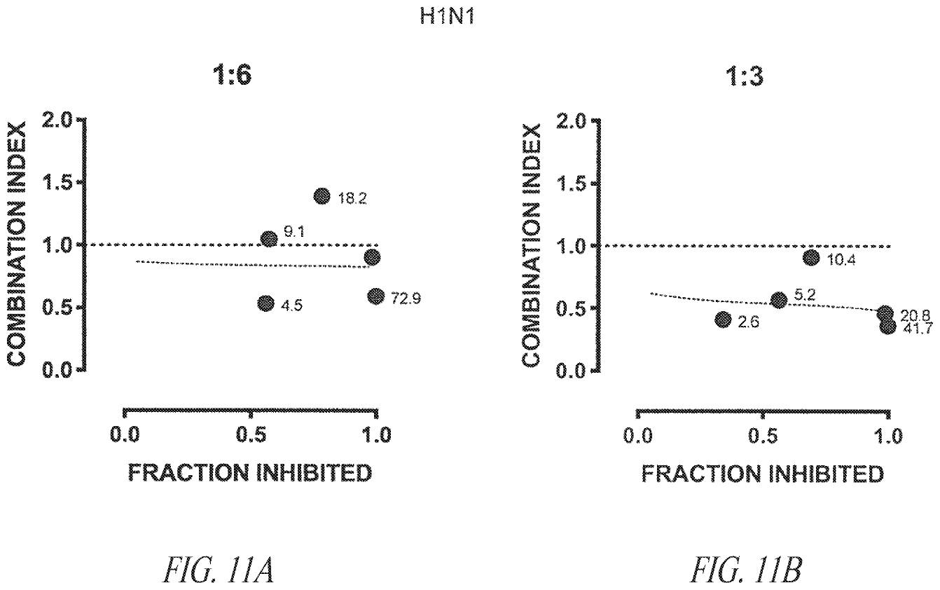

To precisely quantify the putative synergistic effects of the various drug combination ratios, the neutralization data were further transformed according to the median-effect principle and analyzed with the CompuSyn software as described above. The effects of several different FluAB_MLNS-oseltamivir combination constant ratios were plotted in the median-effect plot as shown in FIGS. 10A-10B.

The CompuSyn software applies the logarithmic transformation of the median-effect equation to the experimental data and calculates both the potency (IC₅₀) and the so-called combination index (CI) of the various drug combinations. The CI is a Chou-Talalay (median-effect) equation-derived parameter that considers the physico-chemical properties of the mass-action law and results from the sum of the two ratios between the portion of the dose of drug 1 combined with drug 2 to achieve a certain effect divided by dose of the single drug 1 and 2 to obtain the same effect. According to this mathematical algorithm, a CI=1 indicates an addictive effect, CI<1 indicates synergism and CI>1 indicates antagonism.

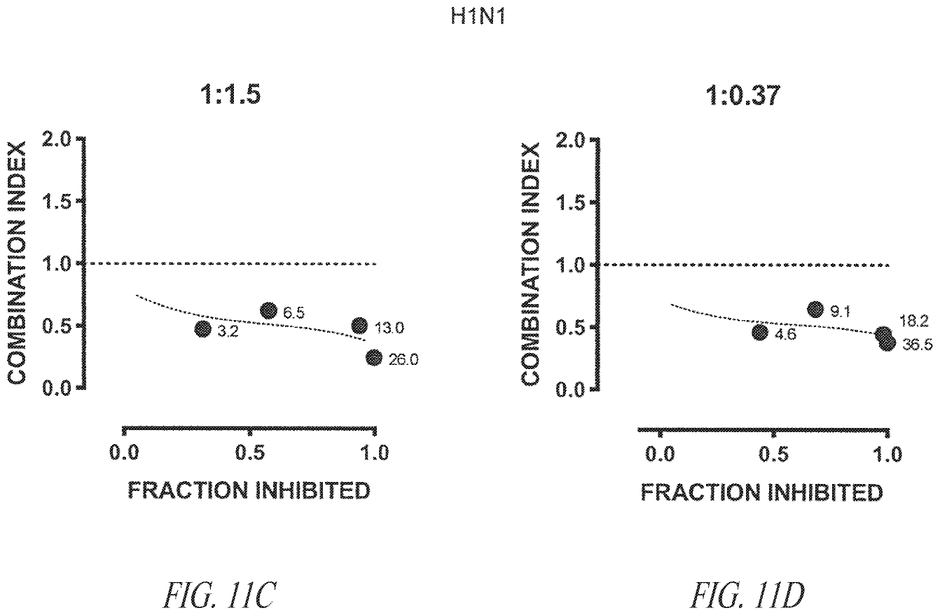

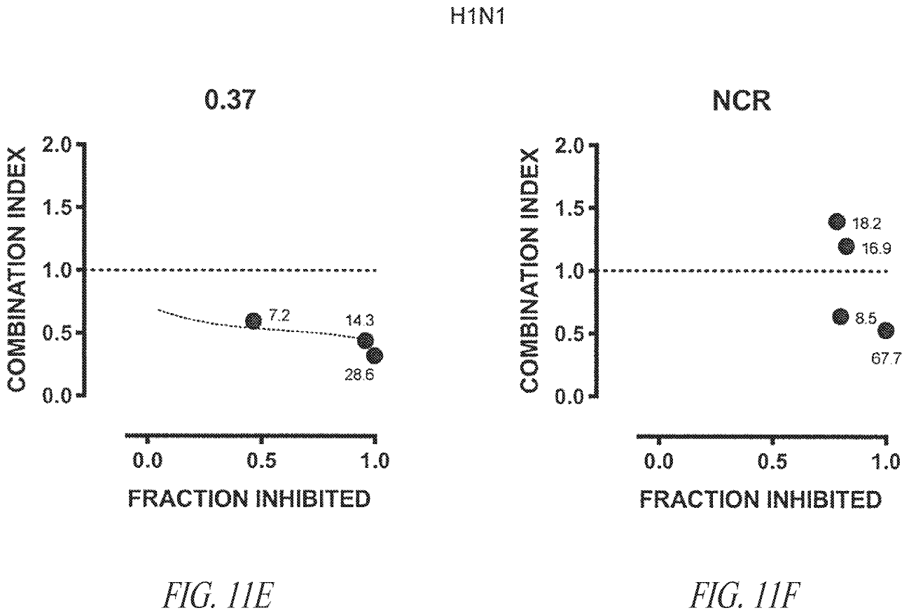

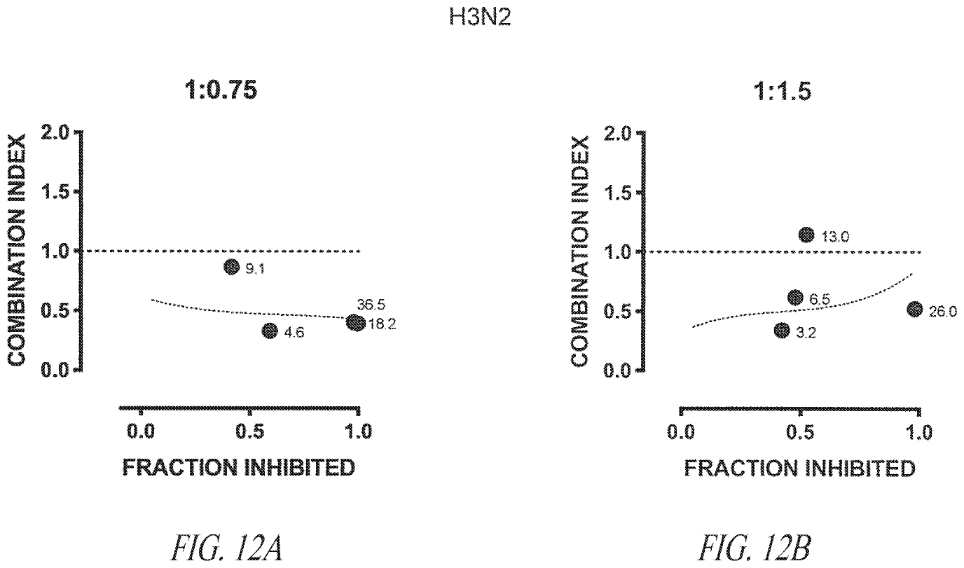

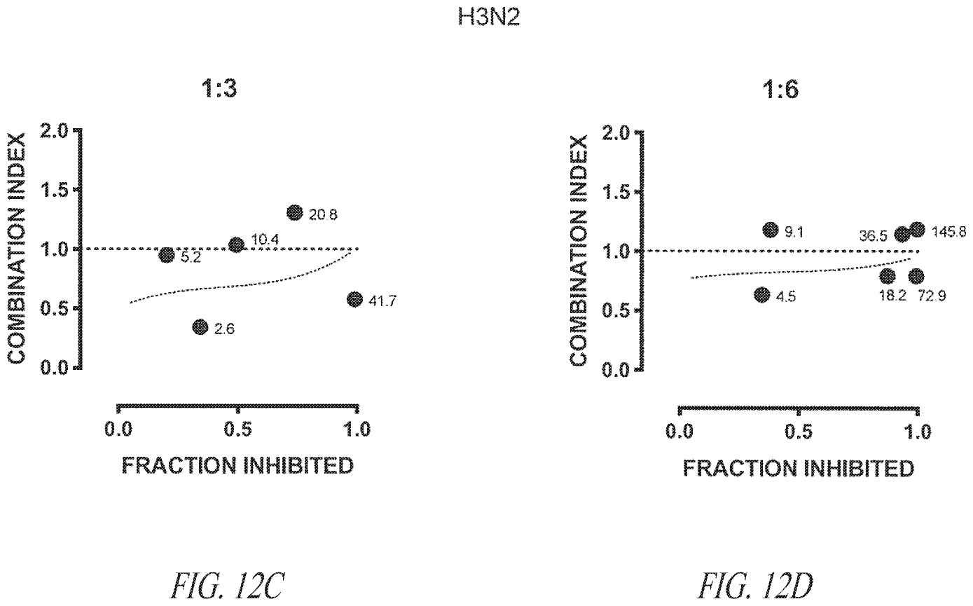

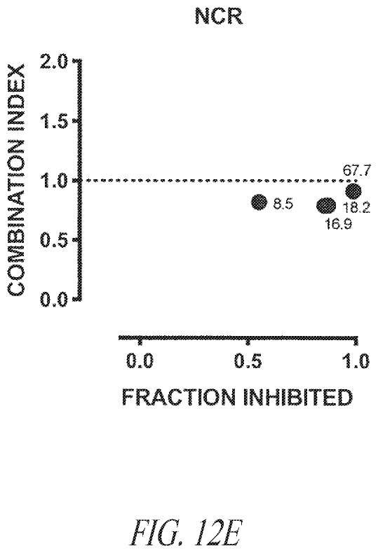

As shown in FIGS. 11A-11F and 12A-12E, for all the combination ratios tested and both for H1 (FIGS. 11A-11F) and H3 virus (FIGS. 12A-12E), the predicted CI values across the inhibited fraction range described a curve well below 1 for all drug combination ratios and the actual experimental points of the different combined concentrations also ranged below 1 for nearly all combinations. Altogether, the data indicate a synergistic effect of FluAB_MLNS and oseltamivir when combined.

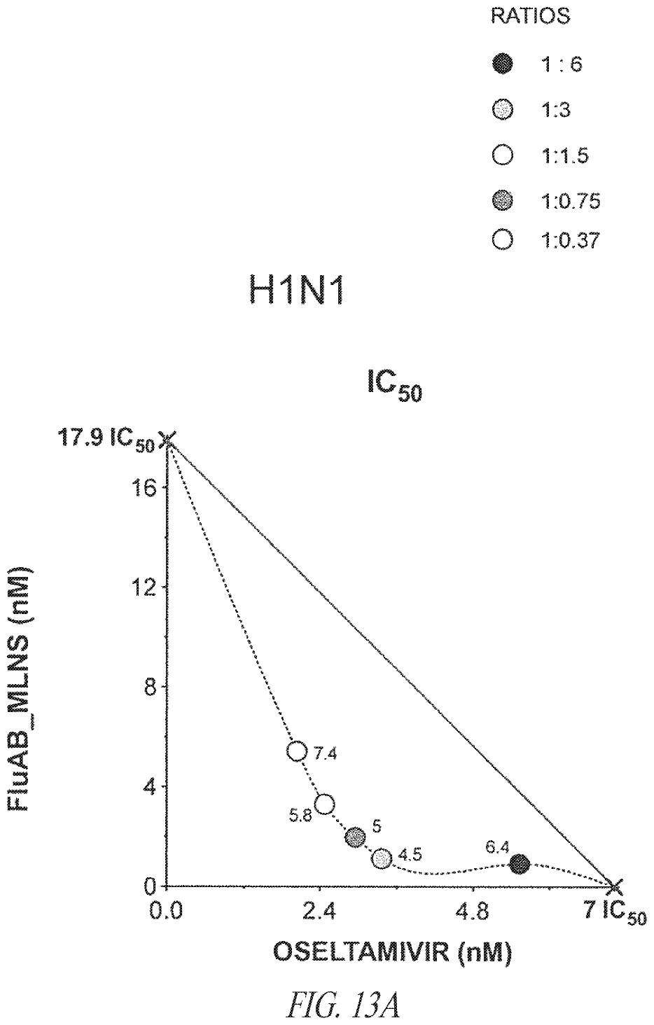

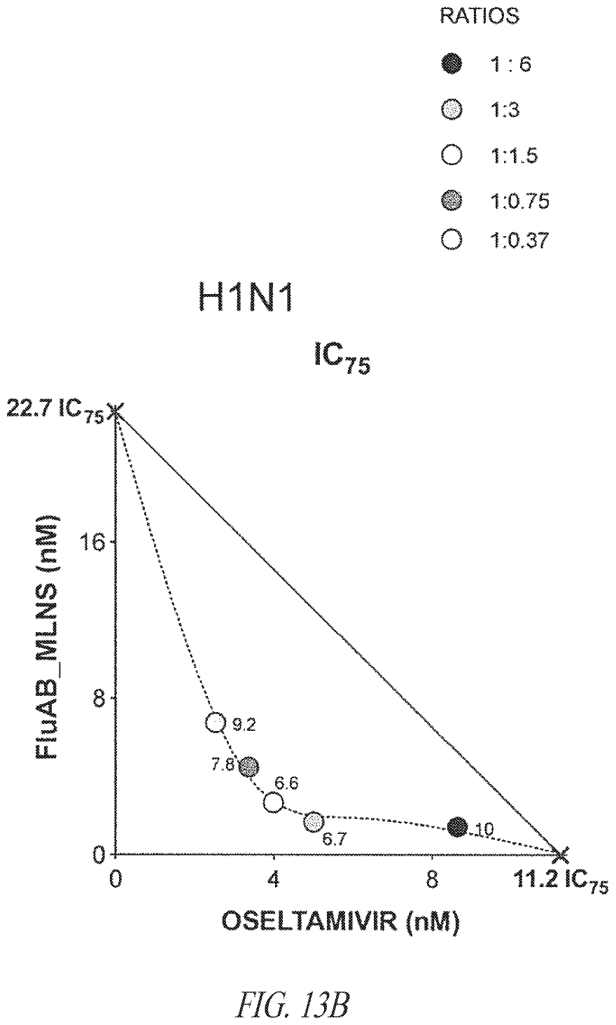

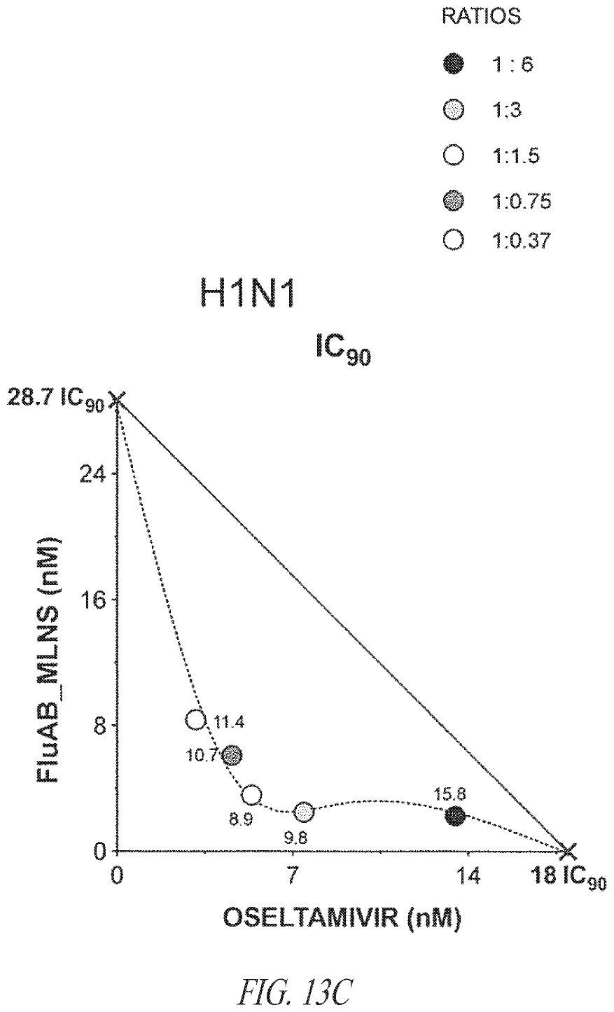

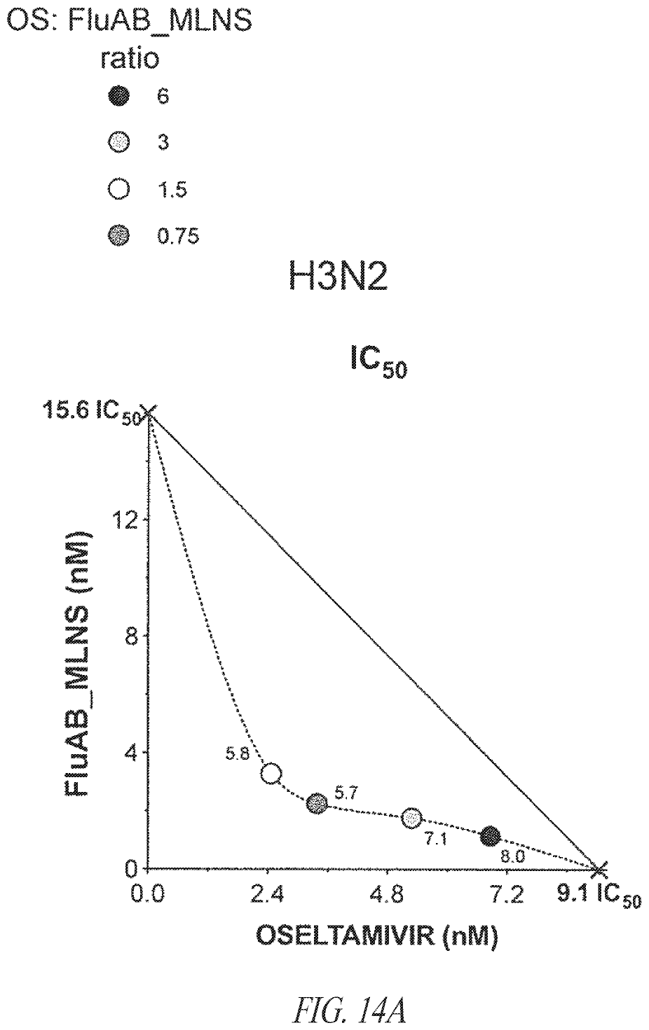

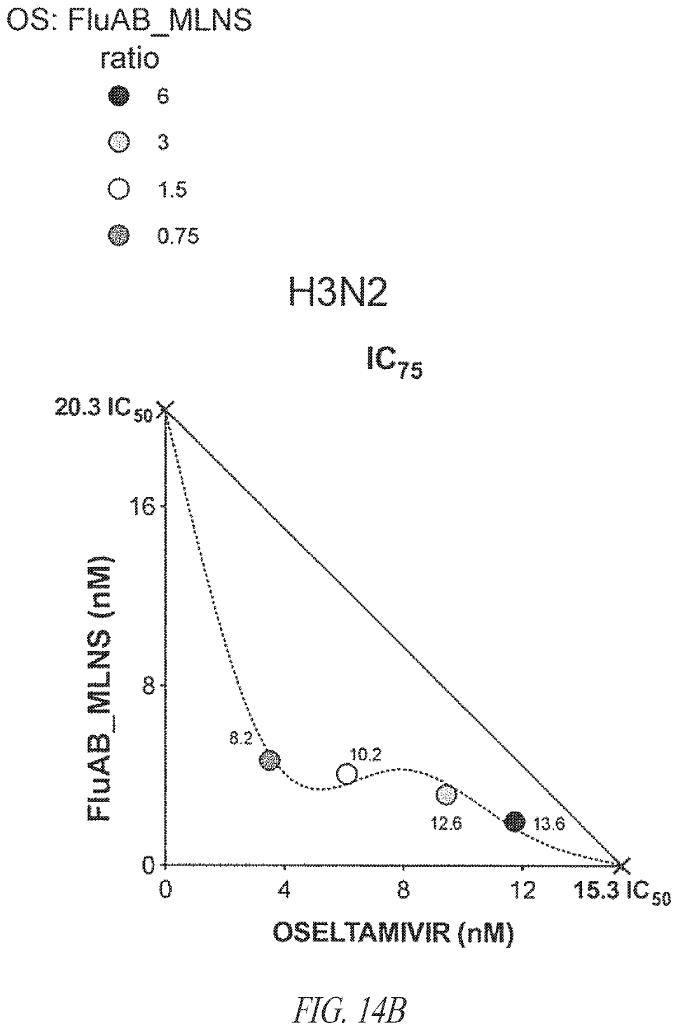

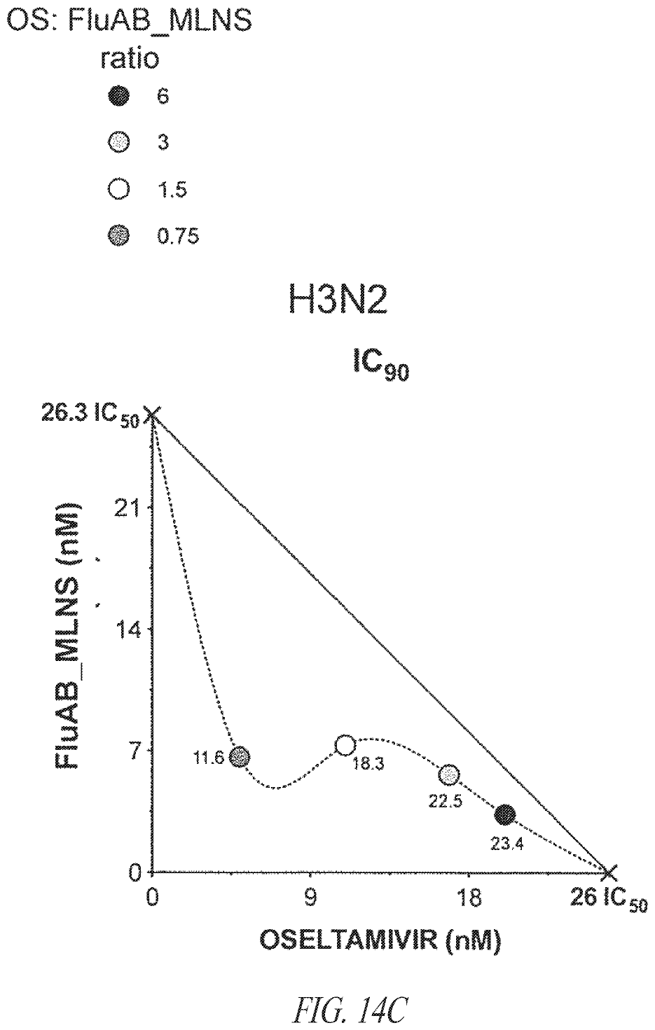

The same data can be alternatively described with isobolograms plots, which compare the equipotent concentrations of both the single and combined drugs. As shown in FIGS. 13 and 14, the distribution of the IC₅₀, IC₇₅, and IC₉₀ values for the three different combination ratios is by far below the isobole lines connecting the respective IC₅₀, IC₇₅, and IC₉₀ of the single drugs tested, both for H1 (FIGS. 13A-13C) and H3 (FIGS. 14A-14C), indicating consistent synergy (while an additive and antagonism would generate equipotency points localized either onto or over the single-drug isobole, respectively).

Combination of FluAB_MLNS and Zanamivir

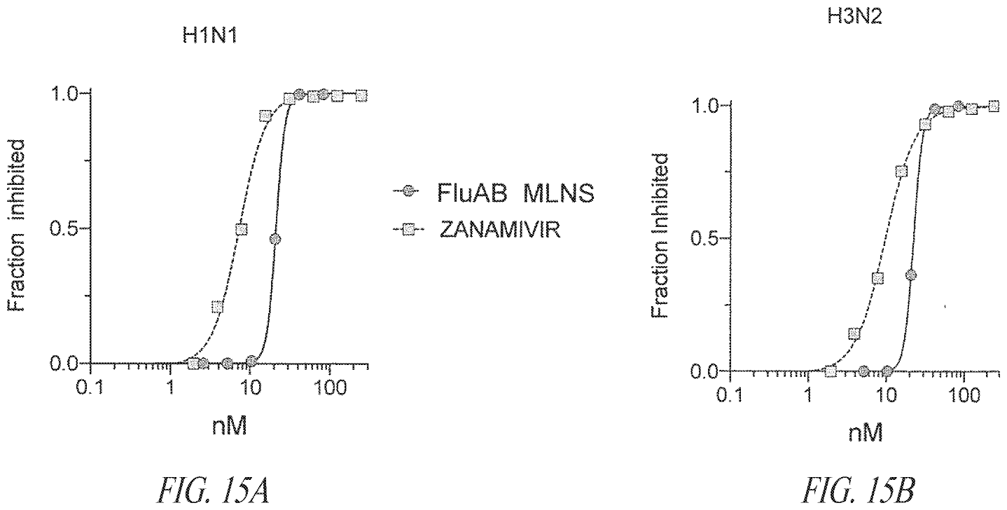

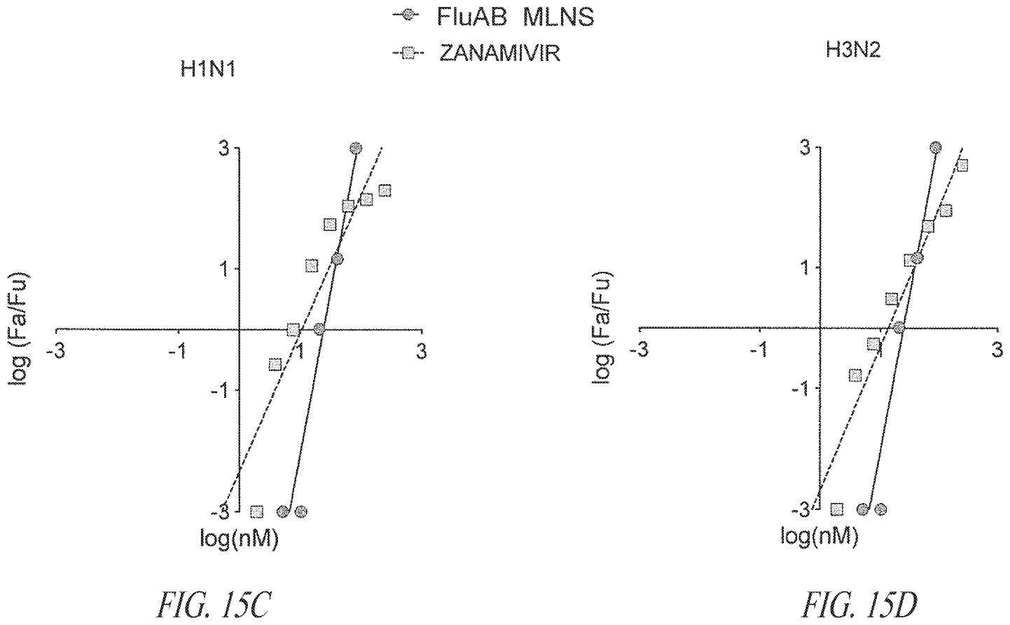

The relative efficacy of FluAB_MLNS and zanamivir to neutralize influenza A viruses was also compared in vitro on two viral serotype representatives for both H3N2 and H1N1 strains. As shown in FIGS. 15A-15D, both compounds, tested separately, were dose-dependently capable to fully inhibit cell infection, when independently exposed together with H3N3 and H1N1 virus. The relative calculated IC₅₀ values were 23.1-24.4 nM for FluAB_MLNS and 10.7-13.7 nM for zanamivir.

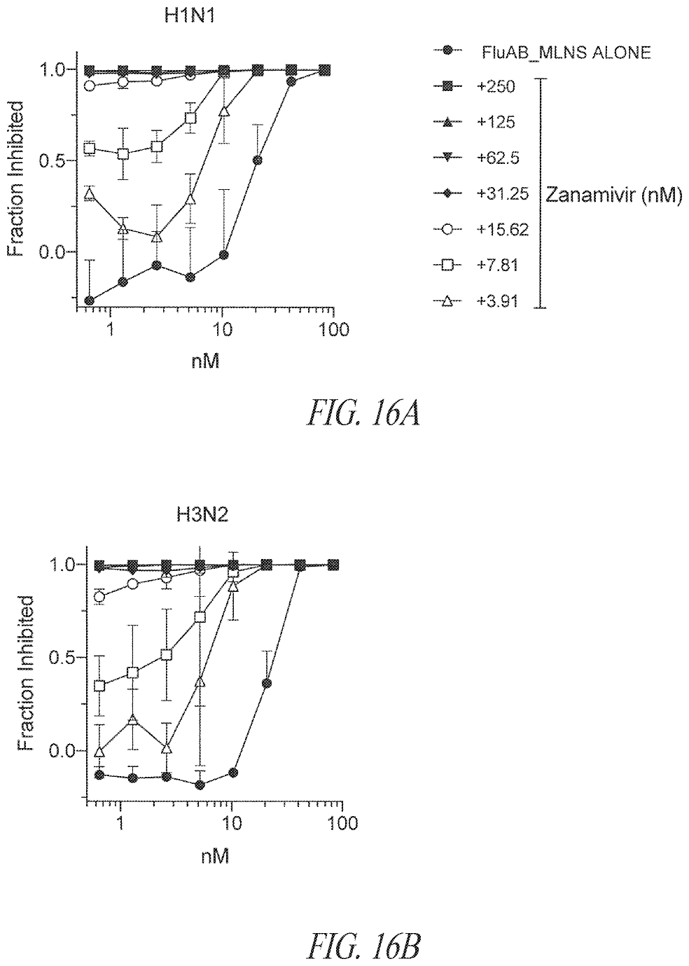

For the combined effect of FluAB_MLNS and zanamivir FIGS. 16A-16B show that, similarly to oseltamivir, zanamivir greatly enhances the inhibitory capacity of FluAB_MLNS both against H1 and H3 viruses.

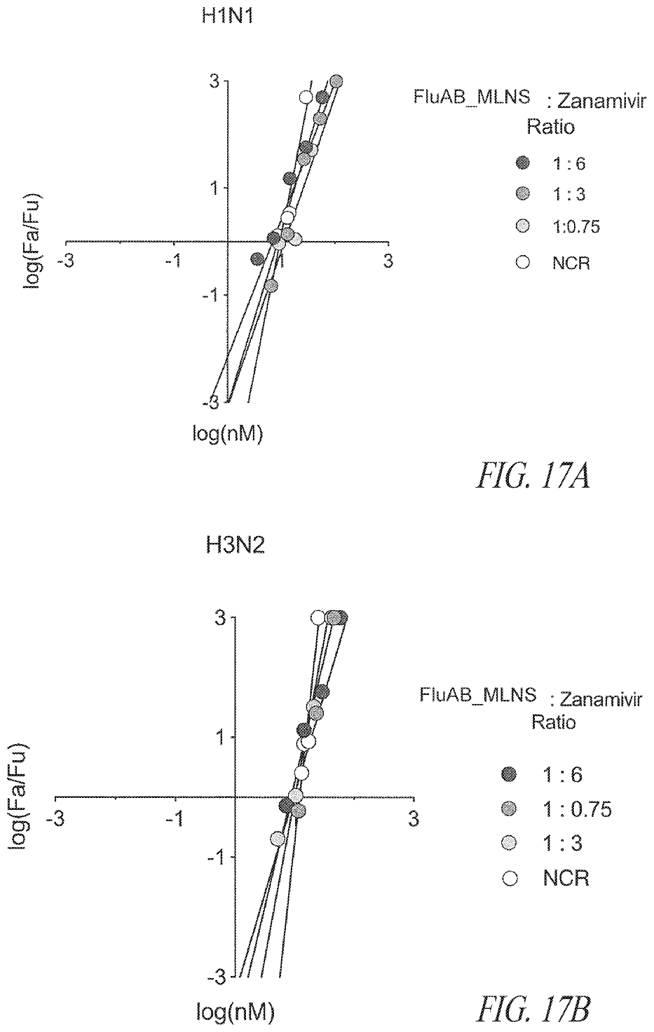

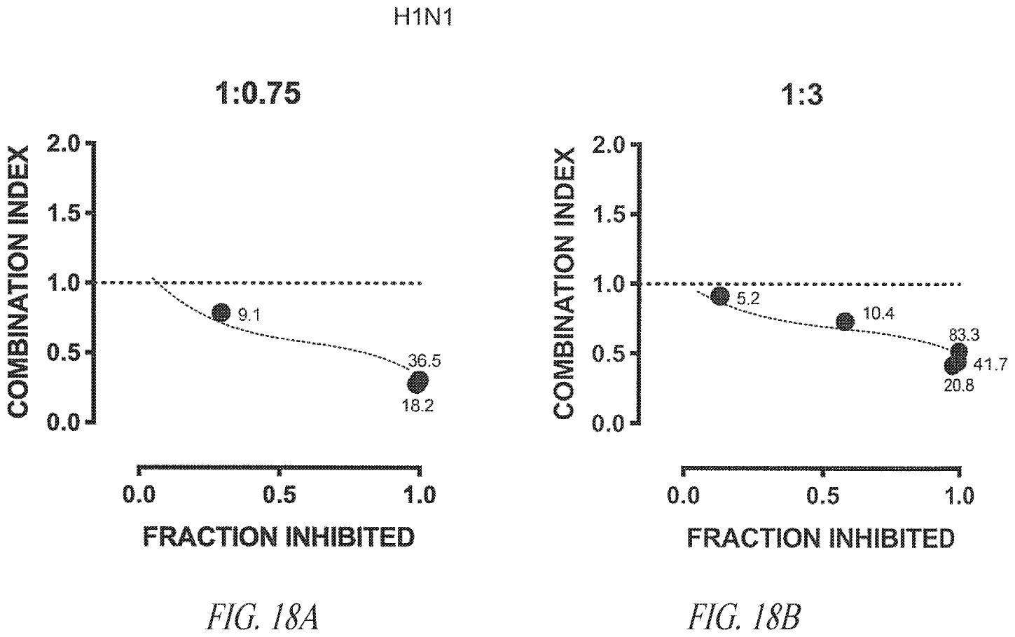

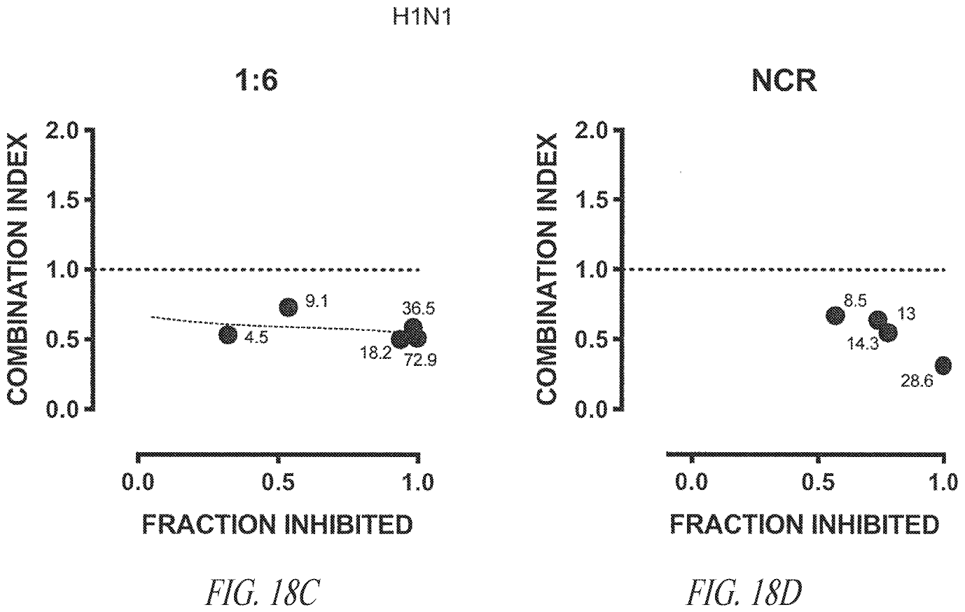

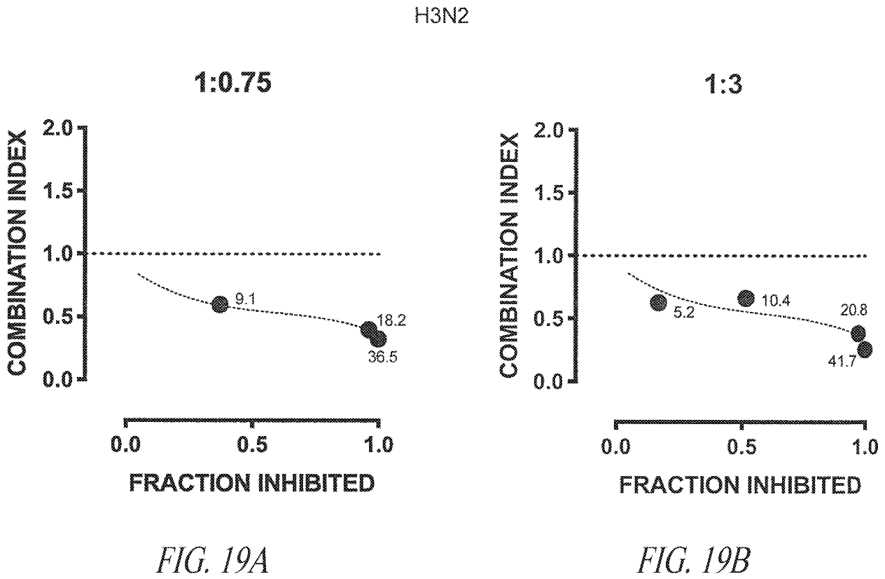

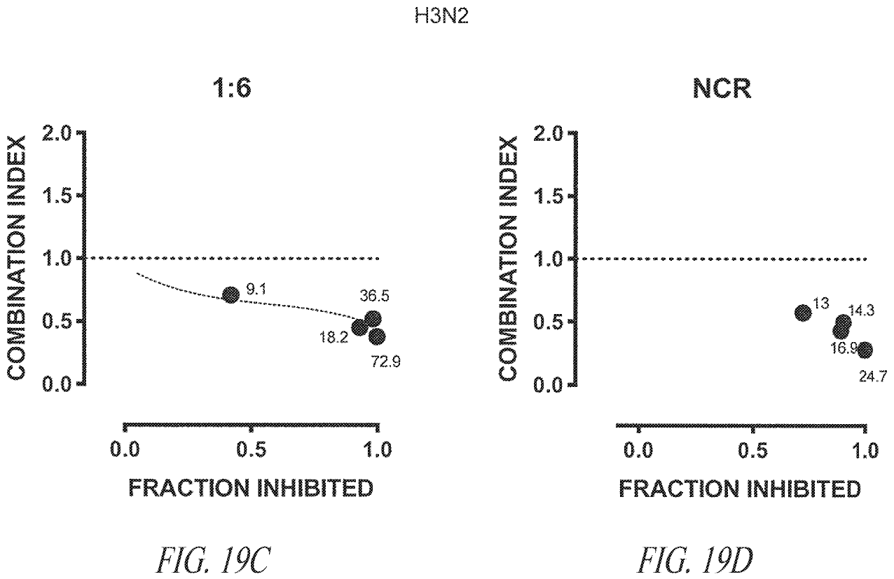

The quantification of the synergic effect was similarly computed with CompuSyn and the median effect principle as described above. The median effect plots for the combined effects of FluAB_MLNS and zanamivir are shown in FIGS. 17A-17B. The calculated CI for FluAB_MLNS and zanamivir is shown in FIGS. 18A-18D and 19A-19D and clearly indicates a synergistic effect between the two drugs, both with H1 (FIGS. 18A-18D) and H3 (FIGS. 19A-19D) viruses, as indicated by the values lower than 1 for all the experimental point tested. Consistently, with both viral strains, the isobolograms denote a strong synergistic effect across the IC₅₀, IC₇₅, and IC₉₀ values (shown in FIGS. 20A-2C and 21A-21C), which are all significantly below the IC values with the single drug.

Combination of FluAB_MLNS and Bloxavir Marboxil

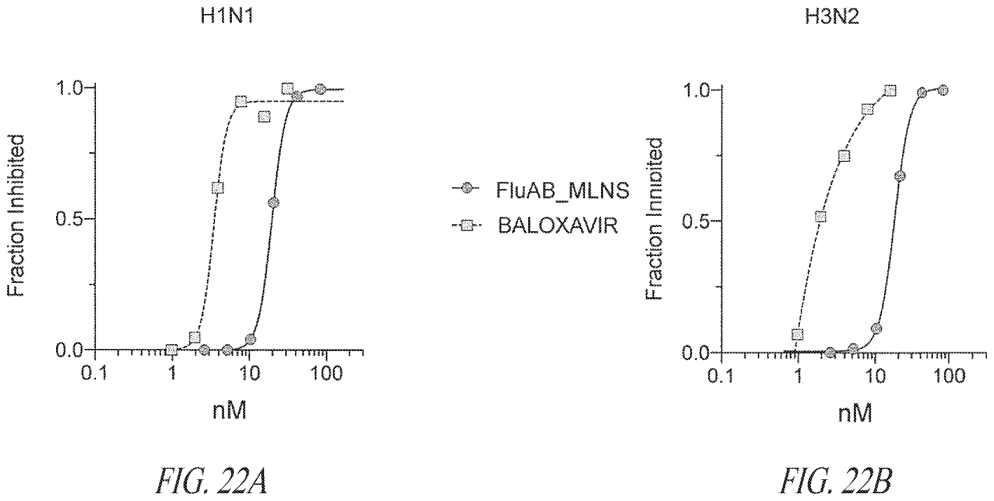

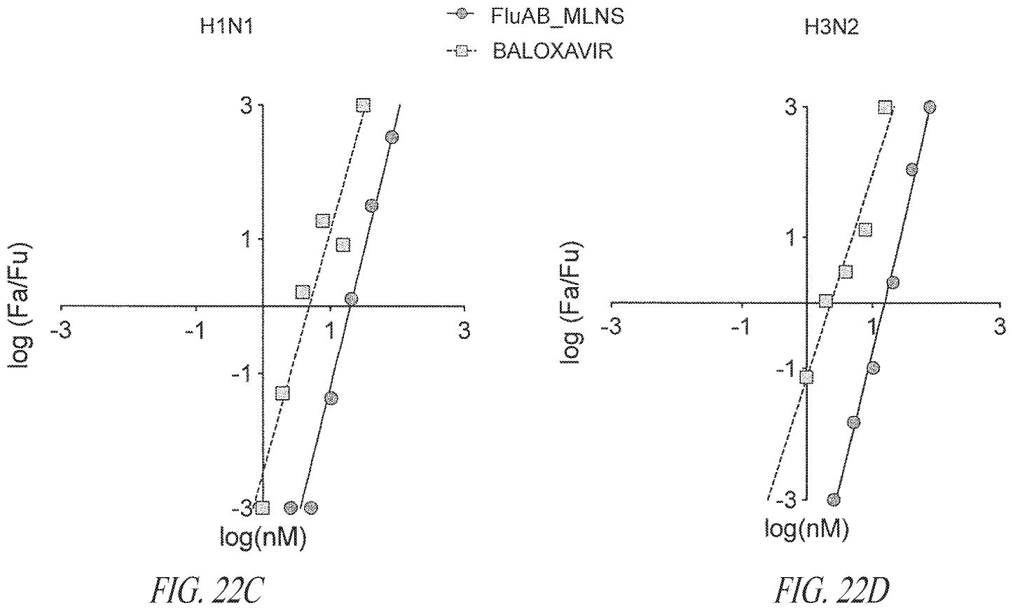

The recently approved endonuclease inhibitor baloxavir marboxil was initially compared with FluAB_MLNS alone on both H1 and H3 strains, similarly as described above for oseltamivir and zanamivir. Results are shown in FIGS. 22A-22D. The relative calculated IC₅₀ values were 20.1-15.4 nM for FluAB_MLNS and 4.9-2.3 nM for baloxavir marboxil.

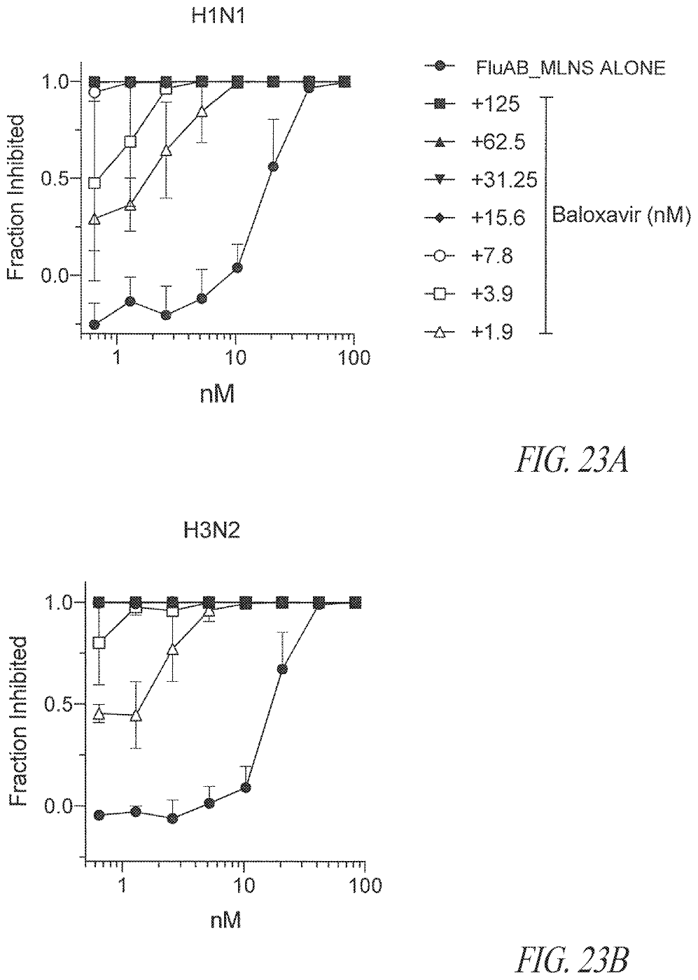

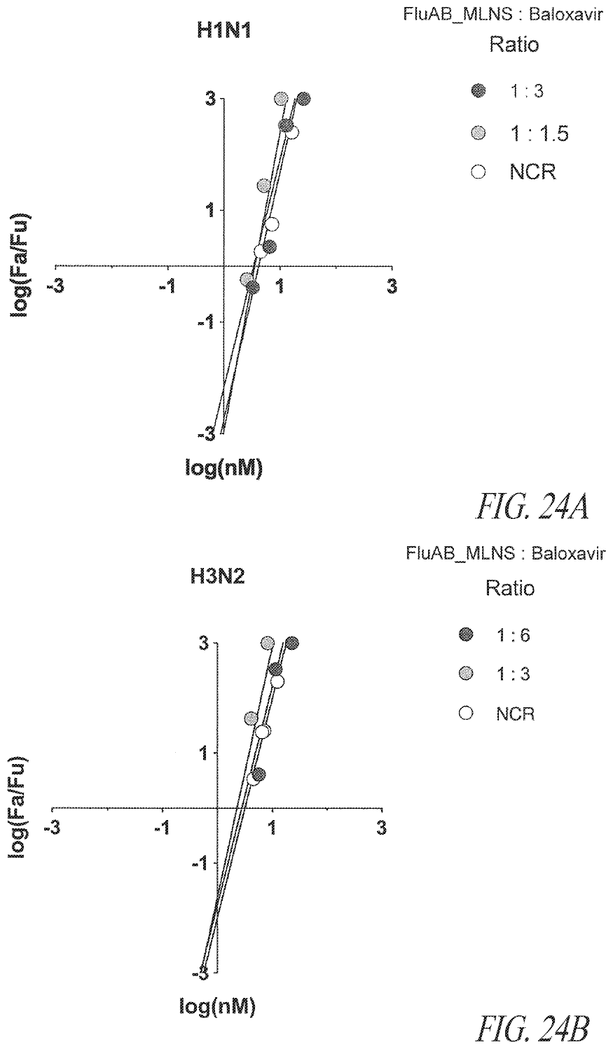

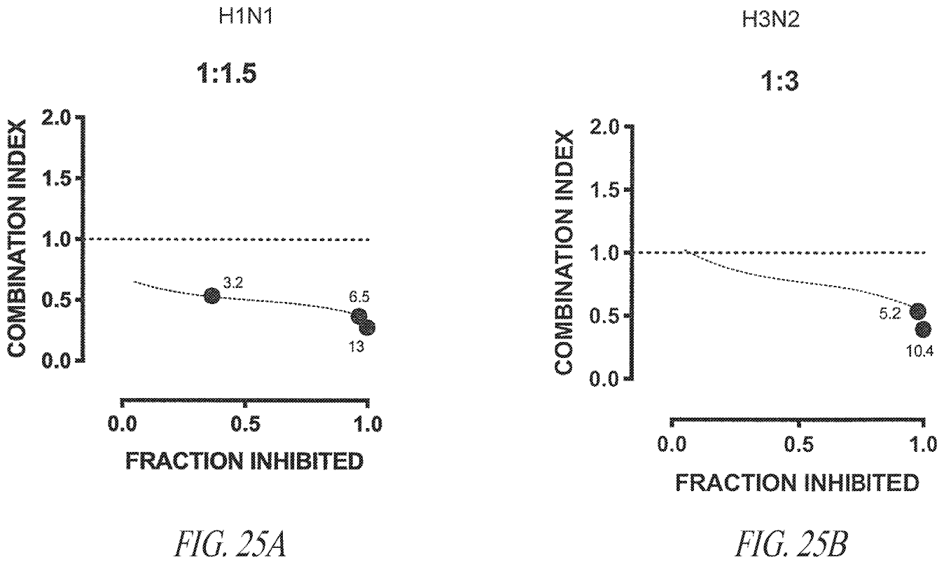

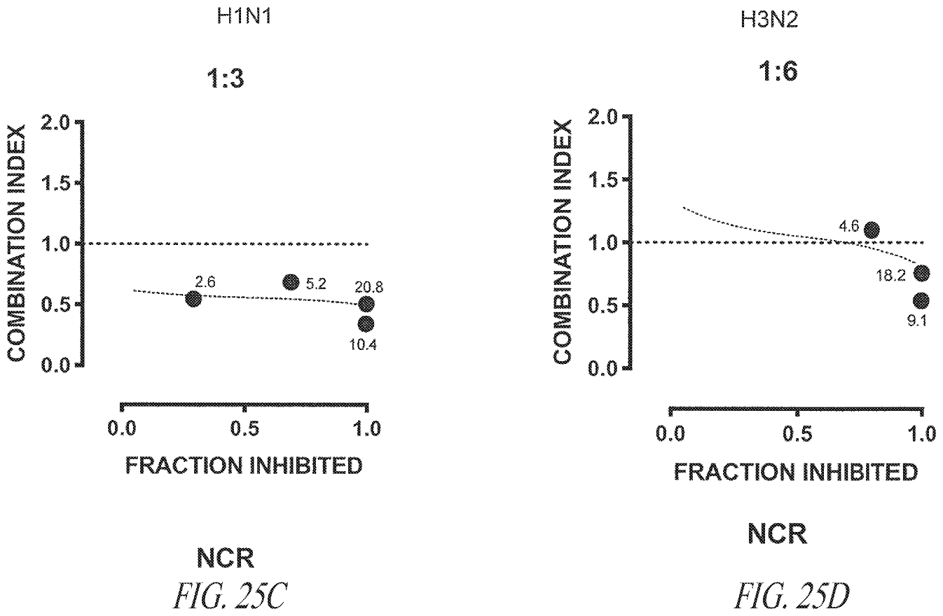

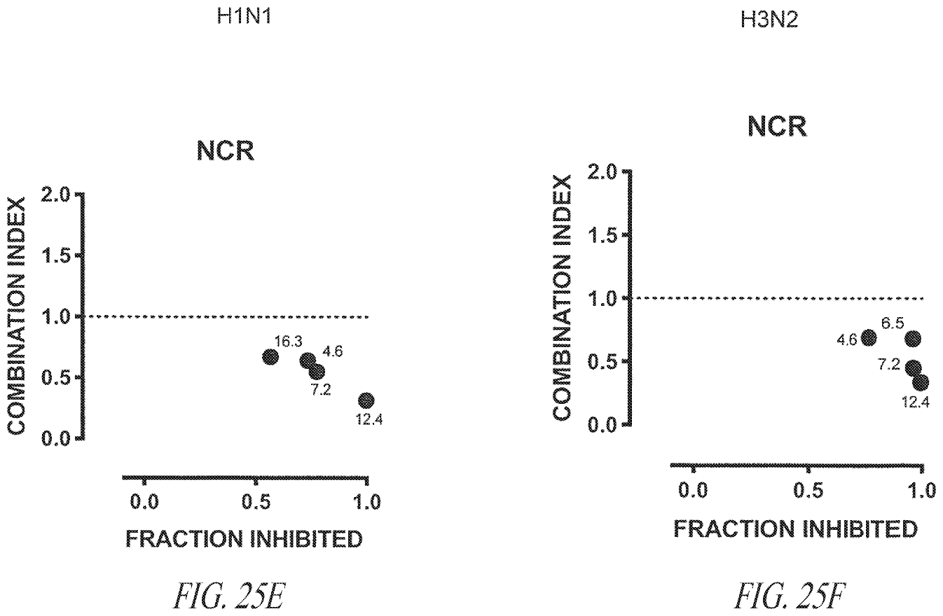

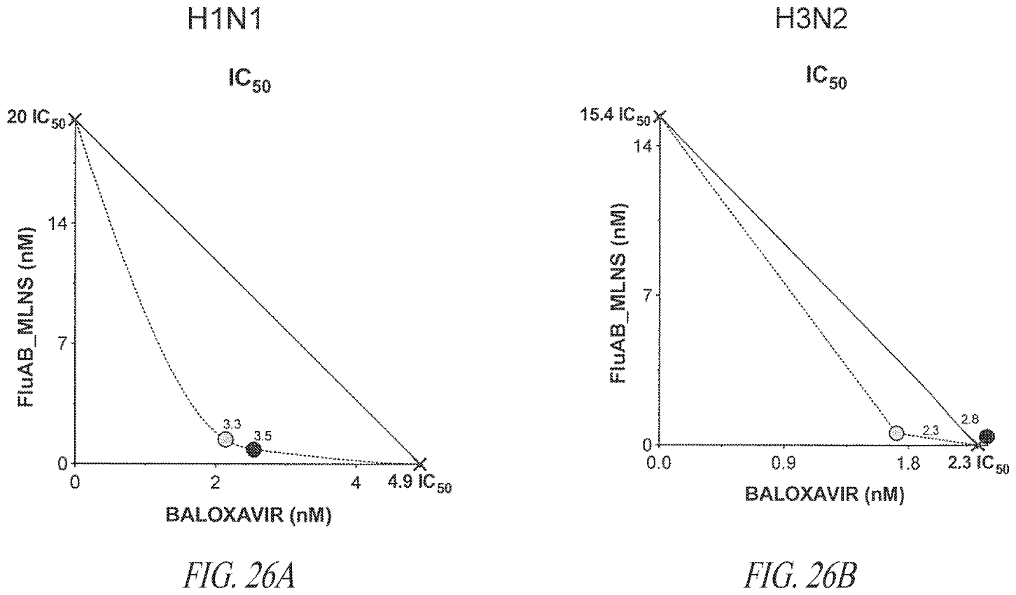

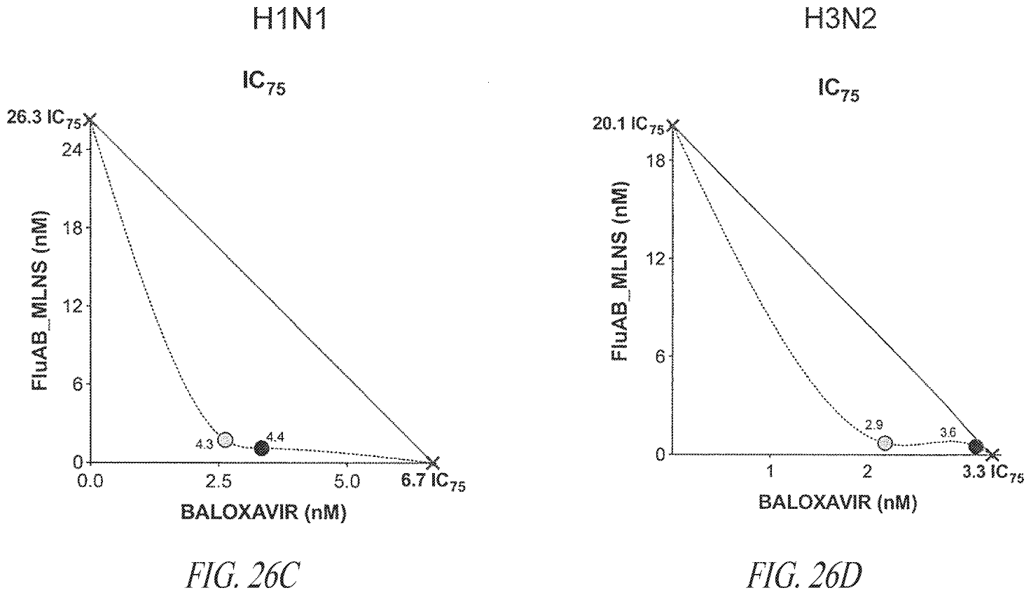

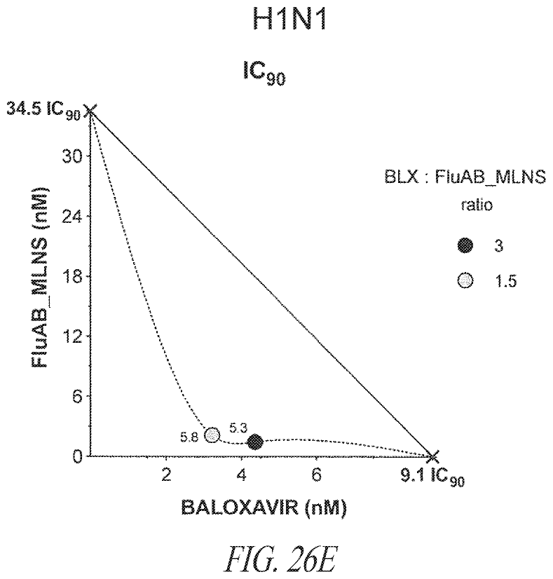

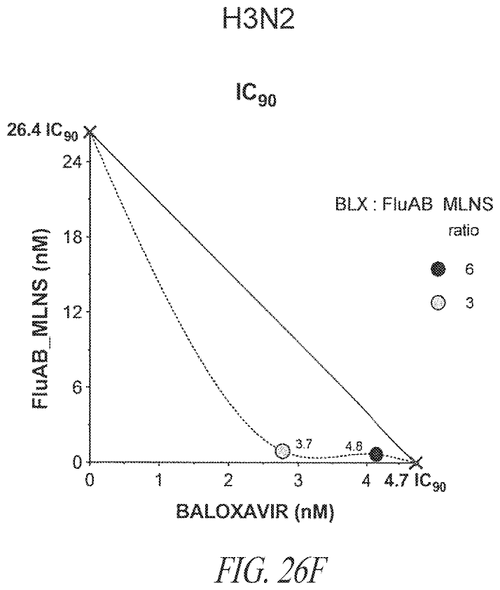

Although Baloxavir has a different mechanism of action in inhibiting viral replication compared to the NA inhibitors, the drug is still able to strongly enhance the inhibitory capacity of FluAB_MLNS, clearly indicating a synergistic effect (FIG. 23A-23B). The inhibition data obtained with the different combination ratios were used to compute and plot the median effect with CompuSyn software and calculate the type of drug-drug interaction as described above (FIGS. 24A-24B). The calculated CI for FluAB_MLNS and baloxavir marboxil (FIGS. 25A-25F) clearly indicate a synergistic effect between the two drugs, both with H1 and H3 viruses, as indicated by the values lower than 1 for the majority of the experimental points tested. The isobolograms denote a robust and complete synergistic effect across the IC₅₀, IC₇₅, and IC₉₀ values (FIG. 26A-26F).

In summary, neutralization capacity of FluAB_MLNS against both H1 and H3 strains is synergistically enhanced by different antivirals, namely, the NA inhibitors oseltamivir and zanamivir as well as the endonuclease inhibitor baloxavir-marboxil.

### Example 7

### Clinical Study of FluAB_MLNS

A phase 1/2, randomized, placebo-controlled study is conducted to evaluate the safety, tolerability, pharmacokinetics, immunogenicity, and efficacy of FluAB_MLNS for the treatment and prevention of Influenza A illness. FluAB_MLNS comprises a light chain amino acid sequence according to SEQ ID NO:10 and a heavy chain amino acid sequence according to SEQ ID NO:9. The study consists of 3 parts:

Part A: A double-blind study to evaluate the safety, tolerability, pharmacokinetics, and immunogenicity of a single ascending dose of FluAB_MLNS in 100 healthy adult subjects.

Part B: An open-label study to evaluate the safety, pharmacokinetics, and immunogenicity of FluAB_MLNS following a second dose given approximately 1 year after the initial dose (i.e., in Part A) in approximately 30-80 subjects from Part A.

Part C: A randomized, double-blinded placebo-controlled study to evaluate the safety, tolerability, efficacy, pharmacokinetics, and immunogenicity of FluAB_MLNS in comparison to placebo for the treatment and prevention of Influenza A illness. The number of subjects is up to 2,760. The study evaluates the effect of FluAB_MLNS compared to placebo on Influenza A-related parameters in subjects with confirmed Influenza-A illness. These parameters include severity of illness, duration of illness, magnitude of viral load in nasopharyngeal secretions at time of presentation with Influenza-A illness. In addition, the study also provides for:
evaluating the effect of FluAB_MLNS on potential biomarkers for host response after Influenza A illness;
monitoring emergence of viral resistance to FluAB_MLNS in subjects presenting with Influenza A illness;
evaluating potential relationships between subject genetic polymorphisms and FluAB_MLNS mechanisms of action and/or pharmacokinetics;
measuring the incidence of healthcare visits due to Influenza A illness;
measuring the impact of FluAB_MLNS on time away from work and productivity due to Influenza A illness.

FluAB_MLNS is evaluated for the prevention of illness due to influenza A in healthy adults at low risk of developing serious influenza-related complications. FluAB_MLNS has high in vitro potency against a wide variety of seasonal and pandemic strains of influenza A virus and is effective in the prevention of otherwise lethal influenza A infection in animal models. In addition, the epitope recognized by FluAB_MLNS is highly conserved among influenza A viruses and has a high barrier to the development of resistance in vitro.

This randomized, placebo-controlled, first-in-human, combined Phase 1/2 study is designed to evaluate the safety, tolerability, pharmacokinetics (PK), and efficacy of FluAB_MLNS for the prevention of influenza A illness in healthy adults without risk factors for serious complications from influenza infection. Parts A and B of the study are designed to collect information on the safety and tolerability of FluAB_MLNS as well as relevant data on the PK profile and the generation of anti-drug antibodies (ADAs). Part C of the study is designed to assess the efficacy of FluAB_MLNS in the prevention of influenza A illness and to collect additional safety, tolerability, and PK data. Potential risks are based on common safety risks observed with mAbs: anaphylaxis and other serious allergic reactions, immune complex disease, and injection site-related reactions.

In the presence of influenza A infection, there is a theoretical risk of antibody-mediated enhancement (ADE) of disease. Subjects are monitored for important potential risks, including assessment of safety in the presence of influenza A infection and routine pharmacovigilance and risk minimization activities is performed.

This study includes healthy adults aged 18 to 64 years of age, without risk factors for serious complications from influenza infection who have not received the upcoming seasonal influenza vaccination.

Placebo is the Control for this Study

To adequately assess the ability of FluAB_MLNS to prevent influenza A illness, study drug administration precedes the influenza season. In this context, data collected during Part A of the study are anticipated to provide sufficient observation of safety and PK to expand into a larger population of healthy adults.

A single fixed dose-escalation range of 60 to 1800 mg (60, 300, 1200, 1800 mg) of FluAB_MLNS administered IM is selected for the study. The recommended highest starting dose in humans is approximately 5 mg/kg or 300 mg fixed dose.

Study Design

This is a randomized, placebo-controlled, Phase 1/2 study of FluAB_MLNS administered intramuscularly (IM) to healthy adult volunteers aged 18 to 64 years of age, who have not received an influenza vaccination for the upcoming season. The study is designed to evaluate the safety, tolerability, PK, immunogenicity, and efficacy of FluAB_MLNS in preventing influenza A illness.

The study is conducted in 3 parts:
Part A: Phase 1, double-blind, single ascending dose of FluAB_MLNS;
Part B: Phase 1, open label, safety and PK following a second dose of FluAB_MLNS;
Part C: Phase 2, double-blinded study to evaluate the efficacy of FluAB_MLNS in prevention of community-acquired influenza A illness.

Data collected during Part A of the study provide observation of safety and PK to expand into a larger population of healthy adults to establish proof of concept. The safety management plan, including SRC oversight in Part A and an independent DMC in Part C, is designed to ensure thorough evaluation of safety data and early detection of potential safety signals.

An interim analysis of safety, efficacy, and futility is conducted when data for approximately half of the first influenza season become available.

In the event subjects experience severe influenza illness, antiviral therapy is offered if deemed appropriate by the Investigator.

Duration of Study Participation

Part A: The estimated total time on study for each subject, inclusive of screening and follow-up, is approximately 13 months.

Part B: The estimated total time on study for each subject is approximately 6 months for Part B and up to 19 months total including Parts A and B together.

Part C: The estimated total time on study for each subject, inclusive of screening and follow-up, is approximately 8 months.

Part A

4 cohorts of 25 subjects each received a single dose of FluAB_MLNS placebo consisting of one (or more) intramuscular (IM) injection(s) to evaluate the safety and tolerability of FluAB_MLNS compared to placebo. The dosing regimens for the 4 cohorts are shown in Table A.

TABLE A
Summary of Part A cohorts

| | Subjects | | FluAB_MLNS Dose or |
|---|---|---|---|
| Cohort | FLUAB_MLNS | Placebo | Matched Placebo |
| 1 | 20 | 5 | 60 mg |
| | | | (1 injection × 0.4 mL) |
| 2 | 20 | 5 | 300 mg |
| | | | (1 injection × 2 mL) |
| 3 | 20 | 5 | Up to 1200 mg |
| | | | (2 injections × 4 mL) |
| 4 | 20 | 5 | Up to 1800 mg |
| | | | (3 injections × 4 mL) |

Eligible subjects were enrolled into 3 sequential cohorts randomized in a 4:1 ratio to receive either FluAB_MLNS or placebo. The first two subjects in each cohort were randomized 1:1 to receive either FluAB_MLNS or placebo. Following 24 hours of sentinel subject observation, all remaining subjects within each cohort were dosed on the same day and remain at the clinical investigative site for 48 hours post-dose.

Local tolerability symptoms that were not resolved on Day 3 are followed until resolution or Day 14 (whichever occurs first). Local tolerability parameters include pain/tenderness, swelling, redness, bruising, and pruritus at the injection site.

Subjects are actively monitored for Influenza-like illness (ILI) throughout the study. Starting on Day 3, subjects complete ILI symptom surveillance questions on an electronic device twice a week. Subjects remain in the study approximately one year to complete assessments for safety, PK, and ADA.

Results:

One hundred (100) subjects received a single dose of FluAB_MLNS (N=80) or placebo (N=20). Preliminary blinded safety data for all cohorts and PK data for the 300 and 1200 mg cohorts were collected.

Serum PK samples were collected at specified visits for 52 weeks. FluAB_MLNS serum concentrations were determined using an electrochemiluminescent method validated on the Meso Scale Discovery (MSD) platform. PK parameters were estimated using standard noncompartmental methods in WinNonlin® 8.2 (Certara L.P., Princeton, N.J.). FluAB_MLNS PK parameters were summarized using descriptive statistics.

Adverse events monitoring, clinical laboratory examination, physical examination and ECG evaluations were performed throughout the study. Injection site tolerability assessment was performed approximately 30 minutes, 2 hrs, 12 hrs, 24 hrs, 48 hrs, and 1 week post-dose.

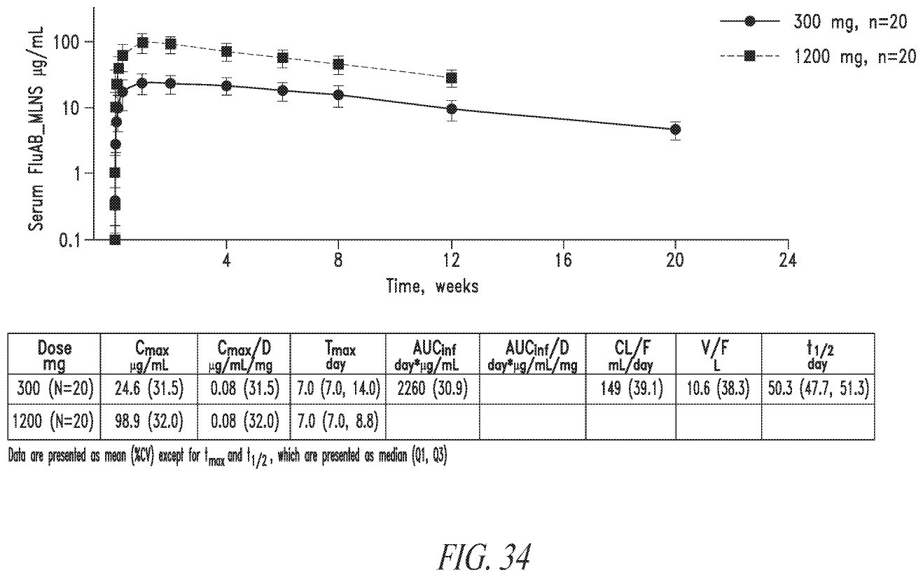

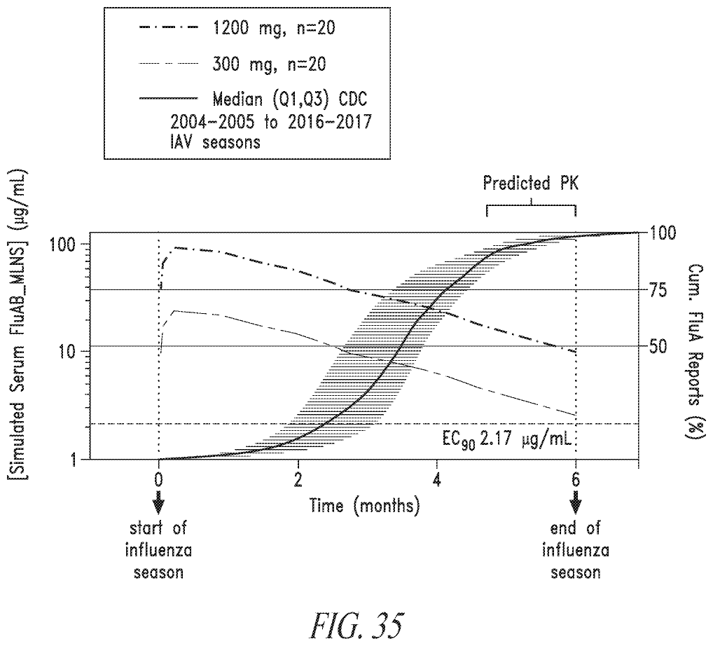

Dosing was well tolerated; 6% ( 6/100) of subjects experienced mild injection site reactions, which generally resolved within 48 hrs. Through 12 weeks post-dosing, the majority (110/112124/126; 98.24%) of adverse events (AEs) were mild to moderate in nature, no serious AEs were reported, and no subjects discontinued due to an AE. Based on available data, exposure (Cmax and AUC) between 300 and 1200 mg of FluAB_MLNS increased in a dose-proportional manner. A PK profile of FluAB_MLNS is shown in FIG. 34, and is consistent with a half-life extended IgG. PK collection is continued through 52 weeks post-dose (e.g., FIG. 35).

FluAB_MLNS was well-tolerated following single IM doses of up to 1800 mg in healthy subjects. The preliminary PK profile of FluAB_MLNS enables once per season dosing.

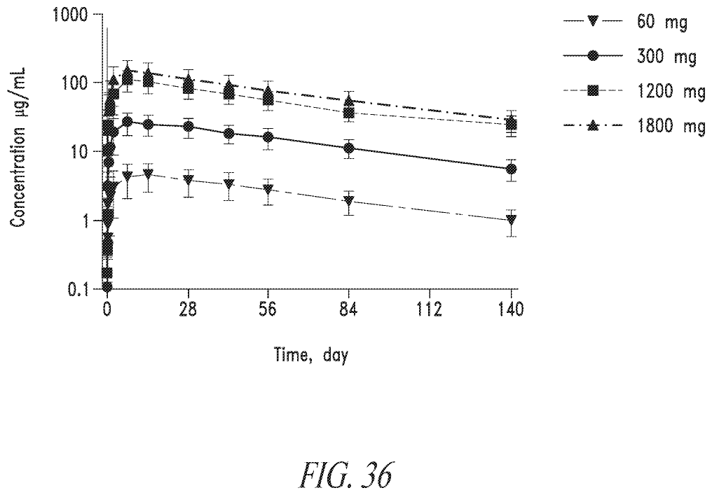

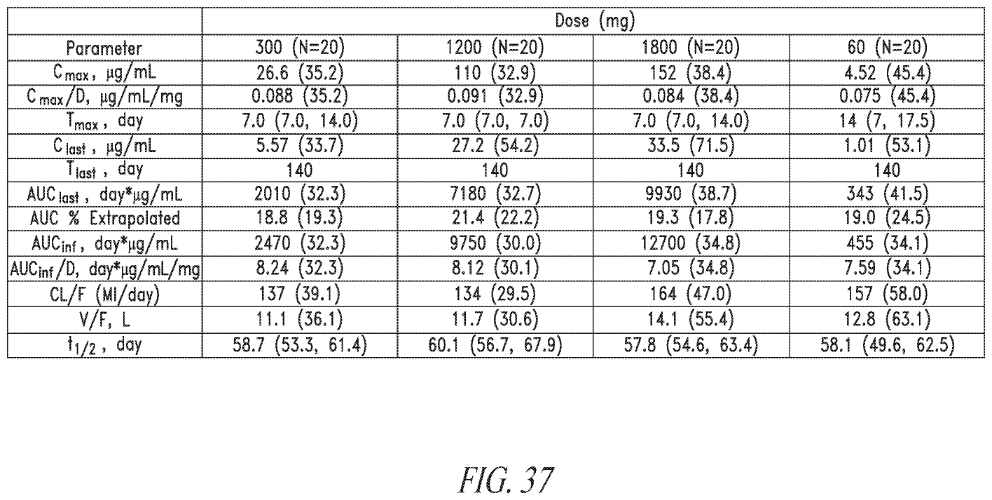

PK data for all dosing groups through Week 20 is shown in FIGS. 36 and 37. An approximate dose-proportional increase in Cₘₐₓ and AUC_{D0-140} was observed, and t_{1/2} is estimated (preliminary) at approximately 58 days.

Part B

Subjects who complete Part A are unblinded at the End of Study visit. Subjects who received FluAB_MLNS have opportunity to consent to participate in Part B. Eligible subjects (≥30) receive a second dose (IM) of FluAB_MLNS approximately 12 months after the initial dose to assess the immunogenicity, safety, and PK of FluAB_MLNS following repeat dosing.

Subjects remain at the clinical investigative site for a minimum of 2 hours post-dose to assess safety and local tolerability of FluAB_MLNS and remain in the study for 24 weeks to complete assessments of safety, PK, and ADA.

Decisions to suspend dosing or discontinue individual subjects from study drug in Part B are made according to predetermined stopping rules. When a stopping rule is met, dosing is paused. Subjects continue to complete symptom surveillance questions on an electronic device twice a week until the end of study to monitor for influenza-like illness (ILI).

Part C

Part C is designed to assess the safety and efficacy of FluAB_MLNS in the prevention of community acquired influenza A illness in healthy adults. Dose selection and enrollment in Part C is initiated after available PK and safety data collected from Part A are reviewed including available safety data through a minimum of 45 days from Cohort 1 and a minimum of 21 days from Cohort 2. Available safety data from Cohorts 3 and 4 are also reviewed.

The PK data from Part A are reviewed to determine dose selection for Part C. Up to two dose levels are evaluated in Part C to allow for exposure-response analyses and dose selection for further Phase 3 studies. Doses selected for evaluation in Part C consider available PK data from Part A in order to maintain a minimum FluAB_MLNS serum concentration of 8 μg/mL for at least 6 months post-dose. Up to 1380 subjects are randomized and enrolled to receive FluAB_MLNS or placebo.

One or two dose levels may be evaluated in Part C. If two dose levels are evaluated, eligible subjects are randomized to receive either FluAB_MLNS or placebo in a 2(n=460):2(n=460):1(n=230):1(n=230) ratio on Day 1. If one dose level is evaluated, subjects are randomized in a 1(n=460):1(n=460) ratio. Each dose level has a matched placebo for number of injections and volume. Subjects remain at the clinical investigative site for a minimum of 2 hours post-dose to assess safety and local tolerability of FluAB_MLNS at injection site(s) and complete assessments.

Subjects are actively monitored for ILI throughout the study. Subjects complete ILI symptom surveillance questions twice a week on an electronic device. Subjects experiencing ILI complete in-clinic evaluations, self-reported influenza symptom severity and WPAI questionnaires, and are followed. Subjects experiencing symptoms consistent with ILI, defined as:
≥1 respiratory symptom (cough, sore throat, rhinorrhea, congestion) AND
≥1 systemic symptom (fever [oral temperature >38° C. (100.4° F.)], chills, myalgias, headache, malaise, fatigue)

For all parts of the study, blood samples are collected to determine presence/absence and titers of anti-drug antibody (ADA. Samples may also be characterized for neutralizing potential of anti-FluAB_MLNS antibody, as appropriate.

All subjects have a follow-up visit approximately 4 and 12 weeks after receiving study drug and approximately 2 weeks after the end of the influenza season for assessment of safety, PK, and ADA. If a dose level requires a second injection of FluAB_MLNS, the second injection is administered at the Week 12 visit. Subjects remain in clinic for a minimum of 2 hours post-dose to assess safety and local tolerability of FluAB_MLNS at injection site(s).

An interim analysis is conducted when data for approximately half of the first influenza season become available for Part C and form the basis for a decision to conclude the study at the end of the season or continue enrollment in a second influenza season.

For Part C, End of Study visit is defined based on the approximate end of flu season in each hemisphere. For participating countries in the Southern Hemisphere, end of flu season is defined as September 30, with End of Study visits completed by approximately mid-October. For participating countries in the Northern Hemisphere, end of flu season is defined as April 30, with End of Study visits completed by approximately mid-May.

For Part C only, blood sample for Fc Receptors for IgG (FcγR) and IgG1 allele genotyping is collected to evaluate potential relationships with FluAB_MLNS mechanism of action or PK. Subjects are provided with an informed consent specifically for genotype assessment.

Subject Population

Subjects are aged 18 to 64 years of age at the time of randomization. Subjects are healthy males and females without acute or chronic medical conditions for Parts A and B. For Part C, subjects must be in good health, determined from a medical history (e.g. any chronic conditions such as hypertension, hyperlipidemia, gastroesophageal reflux disease, anxiety, or depression must be on a stable dose of medication), and no clinically significant finding from physical examination, 12-lead ECG, vital signs, and laboratory values. Body mass index of 18 kg/m² to 32 kg/m² for Parts A and B and 18 kg/m² to 35 kg/m² for Part C. Females must have negative pregnancy test or confirmation of post-menopausal status. Male subjects with female partners of child-bearing potential must agree to use of contraception until the last follow-up visit, or vasectomy with documentation of azoospermia. Patients in Parts A and B are non-smokers. Patients in Part A must agree to abstain from alcohol for 72 hours and caffeine for 24 hours prior to study drug administration. Patients in Part B receive FluAB_MLNS approximately 12 months prior and completed Part A.

Study Procedures

Part A

Screening:

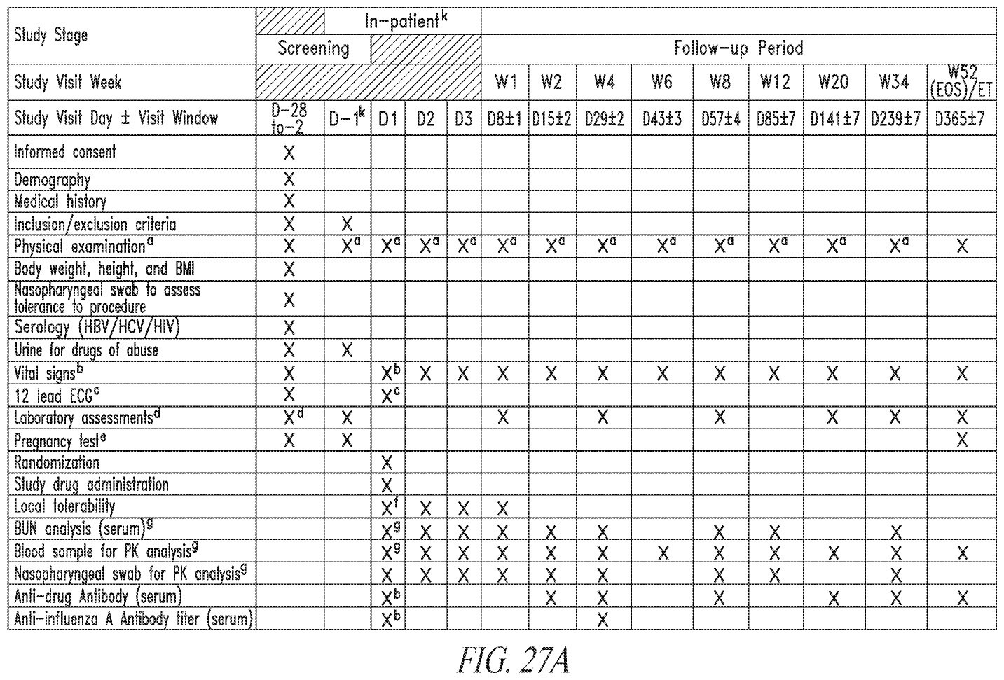

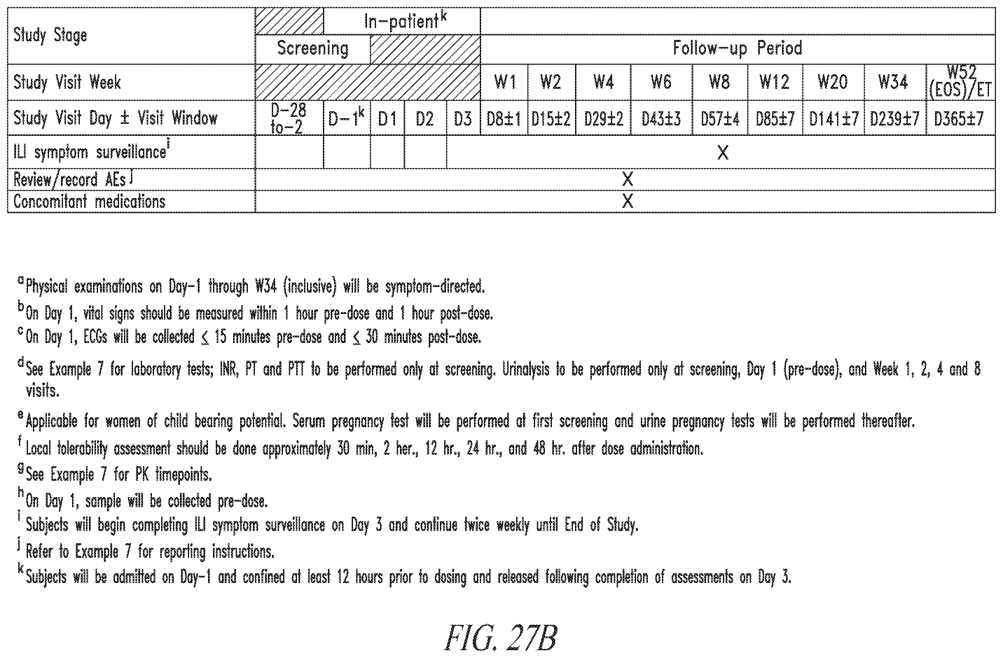

Screening was performed no more than 4 weeks prior to the Day 1 visit and included written informed consent, determination of eligibility, collection of demographics and medical history as well as physical examination (including vitals), laboratory tests, 12-lead electrocardiogram (ECG) and other assessments per the Part A Schedule of Assessments (FIGS. 27A-27B). Inpatient Period (Day —1 to Day 3):

All subjects were admitted to the clinical investigative site on Day −1 and confined for at least 12 hours prior to dosing and for 48 hours following dosing for observation, laboratory evaluation, and PK sampling. Eligible subjects were randomized to receive FluAB_MLNS or placebo on Day 1. Serum and nasopharyngeal PK samples were taken per the Schedule of Pharmacokinetic Timepoints (FIG. 31). Subjects were discharged after all study assessments were performed on Day 3.

Follow-Up Period:

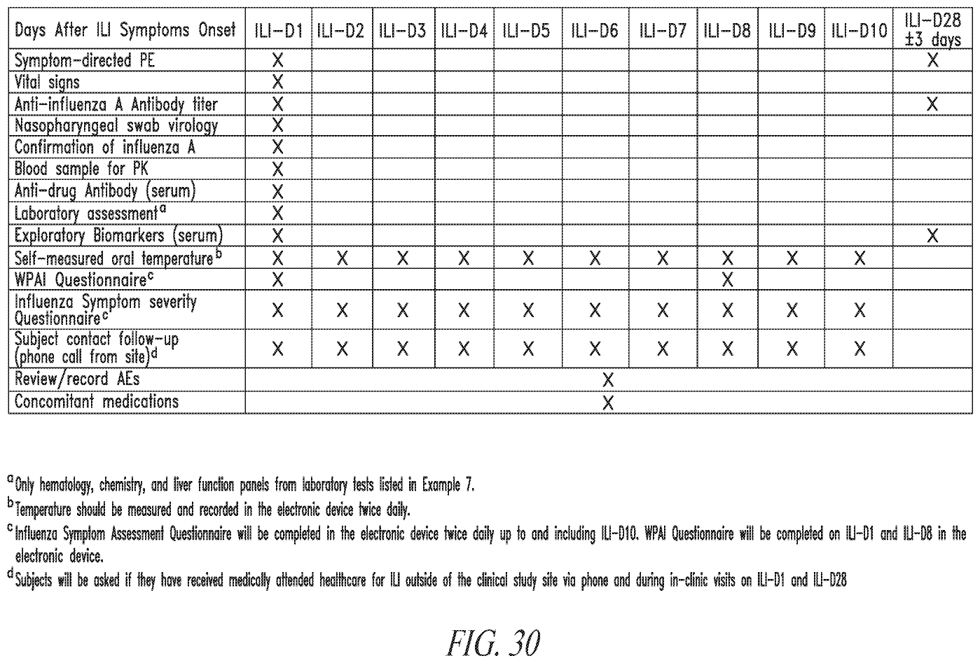

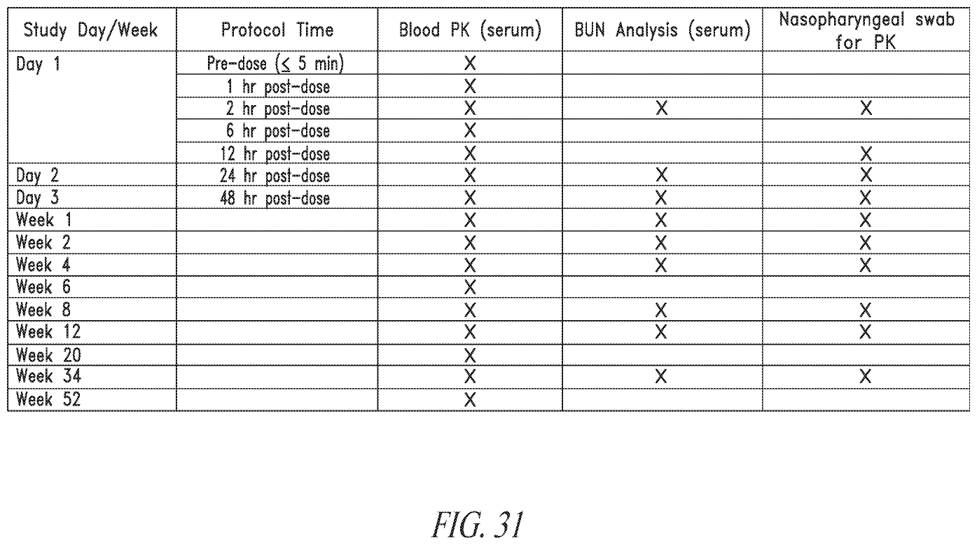

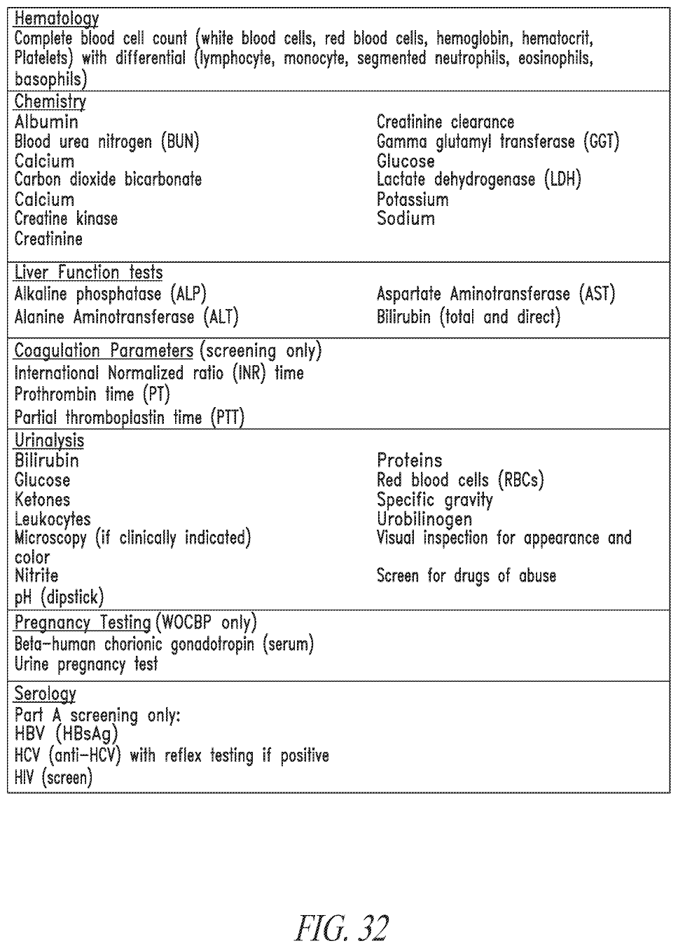

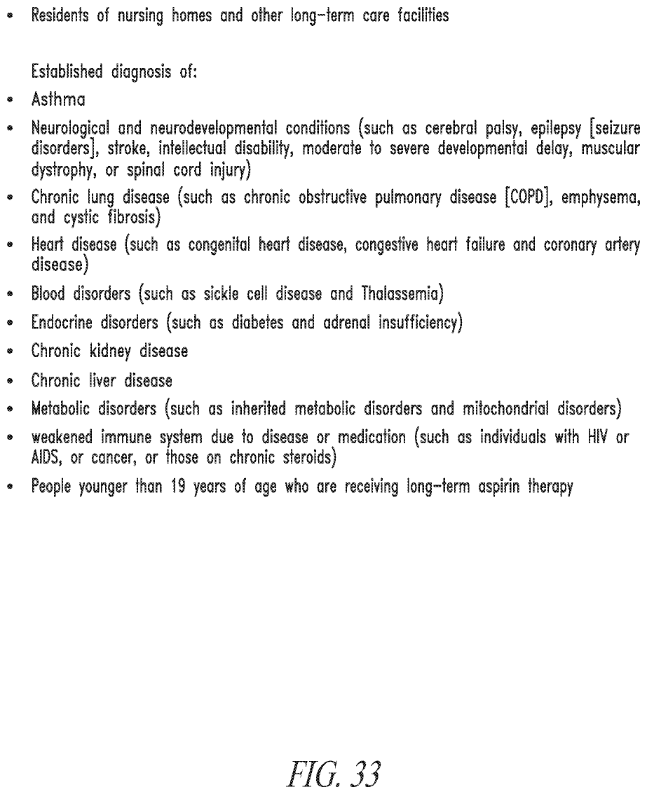

Subjects returned to the clinical investigative site for in-person assessments per the Part A Schedule of Assessments (FIGS. 27AA-27B) including, but not limited to, physical examination (including vitals), 12-lead ECG, laboratory testing (including safety, PK, and ADA), review of AEs and concomitant medications, as appropriate. Serum and nasopharyngeal PK samples were taken per the Schedule of Pharmacokinetic Timepoints (FIG. 31). Subjects completed ILI symptom surveillance questions on an electronic device twice a week through the End of Study. If subjects experienced ILI, they were monitored under the same provisions as Part C, per the Influenza-like Illness Monitoring Schedule (FIG. 30).

Part B

Screening:

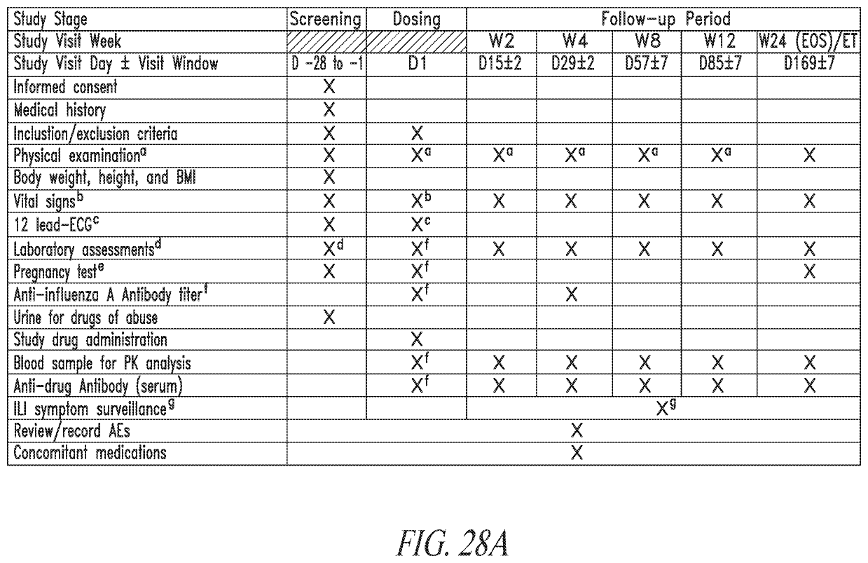

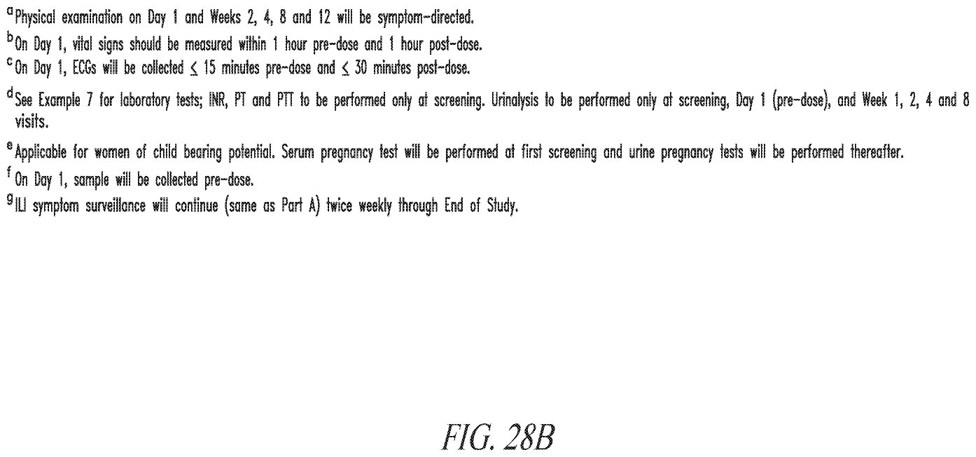

Provided evaluations of safety, PK and efficacy data from Parts A and C of the study support further product development, subjects who received FluAB_MLNS may be able to progress to Part B. Subject randomization is unblinded at the Part A End of Study visit and subjects who received FluAB_MLNS may consent to be evaluated for eligibility to receive a second dose of FluAB_MLNS approximately 12 months after the initial dose. Screening assessments per the Part B Schedule of Assessments (FIGS. 28A-28B) are performed no more than 4 weeks prior to the Day 1 visit.

Dosing (Day 1):

Eligible subjects receive a dose FluAB_MLNS on Day 1. Subjects remain in the clinic for a minimum of 2 hours post-dose to assess safety and local tolerability of FluAB_MLNS and complete assessments per the Part B Schedule of Assessments (FIGS. 28A-28B).

Follow-Up Period: Subjects return to the clinical investigative site for in-person assessments per the Part B Schedule of Assessments (FIGS. 28A-28B) including, but not limited to, physical examination (including vitals), laboratory testing (including safety, PK, and ADA), review of AEs and concomitant medications as appropriate. Subjects complete ILI symptom surveillance questions on an electronic device twice a week through End of Study. Subjects experiencing ILI are monitored under the same provisions as Part C, per the Influenza-like Illness Monitoring Schedule (FIG. 30).

Part C

Screening:

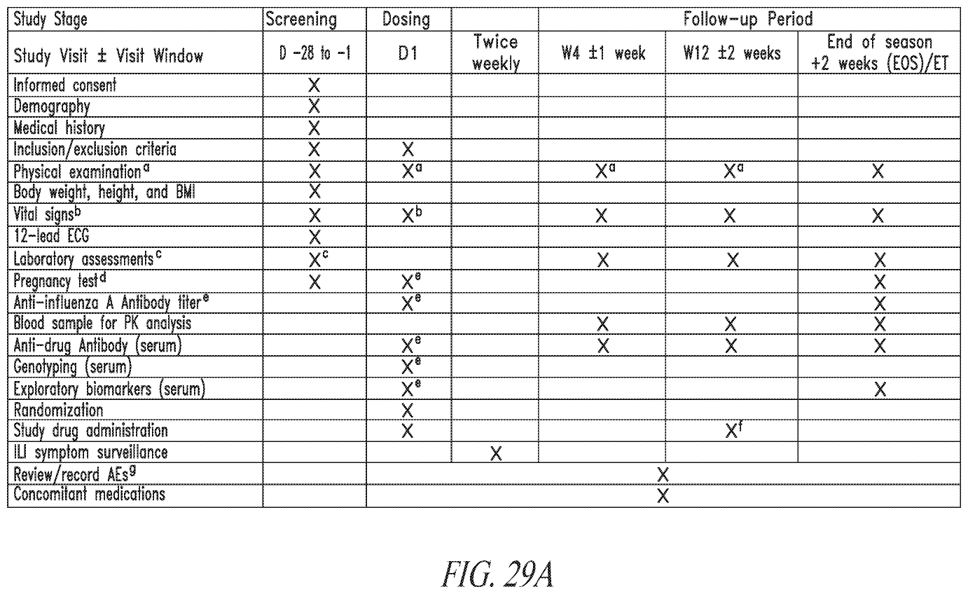

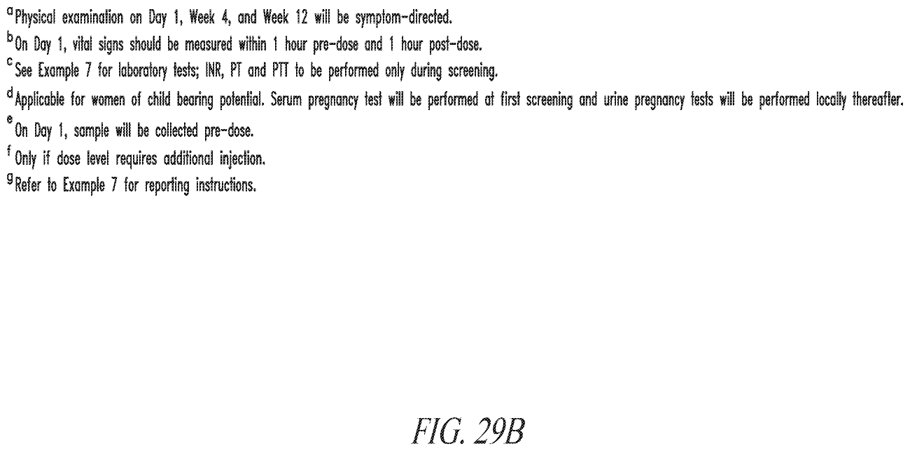

Screening is performed no more than 4 weeks prior to the Day 1 visit and includes written informed consent, determination of eligibility, collection of demographics and medical history as well as physical examination (including vitals), laboratory tests, 12-lead electrocardiogram (ECG) and other assessments per the Part C Schedule of Assessments (FIGS. 29A-29B).

Dosing (Day 1):

If two dose levels are evaluated, eligible subjects are randomized to receive either FluAB_MLNS or placebo in a 2(n=460):2(n=460):1(n=230):1(n=230) ratio on Day 1. If one dose level is evaluated, subjects are randomized in a 1(n=460):1(n=460) ratio. If the chosen dose level requires a second injection of FluAB_MLNS, the second injection is administered at the 12-week visit. Each dose level will have a matched placebo for number of injections and volume. Subjects remain at the clinical investigative site for a minimum of 2 hours post-dose to assess safety and local tolerability of injection site(s) and complete assessments per the Part C Schedule of Assessments (FIGS. 29A-29B).

Follow-Up Period:

Subjects return to the clinical investigative site for in-clinic assessments per the Part C Schedule of Assessments (Appendix 4). Subjects complete ILI symptom surveillance questions on an electronic device twice a week through the End of Study. Subjects experiencing symptoms consistent with ILI, defined as ≥1 respiratory symptom (cough, sore throat, rhinorrhea, congestion) AND ≥1 systemic symptom (fever, chills, myalgias, headache, malaise, fatigue) during follow-up should report symptoms on same day as onset and arrange for an in-clinic evaluation per the Influenza-like Illness Monitoring Schedule (FIG. 30).

In-clinic evaluation includes, but is not limited to, physical examination (including vital signs), laboratory testing (including safety), nasopharyngeal swab for virology, blood samples for PK and ADA, and review of AEs and concomitant medications as appropriate. Subjects also self report influenza symptom severity and complete the WPAI questionnaire per the Influenza-like Illness Monitoring Schedule (FIG. 30). The WPAI is a validated, patient-reported, quantitative assessment of absenteeism (work time missed), presenteeism (reduced on-the-job effectiveness), work productivity loss and activity impairment due to a specific health problem. Subjects who present with ILI complete the 6-item WPAI questionnaire on ILI-D1 and ILID8 (see FIG. 30).

Influenza Symptom Severity Questionnaire

Subjects perform a self-assessment of systemic and respiratory symptoms associated with influenza (i.e., cough, sore throat, headache, nasal congestion, feverishness or chills, muscle or joint pain, and fatigue). They score the severity of their symptoms using a 4-point rating scale (0, None; 1, Mild; 2, Moderate; 3, Severe). This information is captured in an electronic device twice daily from ILI-D1 to ILI-D10 (10 days inclusive) during the Influenza-like Illness Monitoring period (FIG. 30).

Anti-influenza A Antibody Titer

In all parts of the study, blood samples are collected to determine anti-influenza A antibody titers using standard methods according to Part A, B and C Schedule of Assessments (FIGS. 27A/27B, 28, and 29, respectively) and the Influenza-like Illness

Monitoring Schedule in FIG. 30. Details regarding the processing of the samples are provided in the Laboratory Manual.

Resistance Surveillance

In all parts of the study, resistance surveillance for potential emergence of resistance to FluAB_MLNS are conducted for all subjects who received study drug and present with laboratory confirmed influenza A virus infection, as per the Influenza-like Illness Monitoring Schedule (FIG. 30). Nasopharyngeal swab samples from subjects with confirmed influenza A virus infection are subjected to deep sequencing analysis of the HA gene to determine amino acid variants. To evaluate the emergence of antiviral resistance, virus culture is attempted on subjects with laboratory confirmed influenza A virus infection and in vitro phenotypic analysis of the antiviral activity of FluAB_MLNS on virus cultured from subjects with confirmed IAV is attempted. For subjects where IAV is unable to be cultured, recombinant virus containing the HA gene from influenza A virus-positive subjects is generated using established reverse genetic procedures, and recombinant viruses are subjected to phenotypic analysis.

Exploratory Biomarkers

For Part C and during ILI Monitoring, samples will be collected per the Schedule of Assessments (FIGS. 29A-29B and 30, respectively) to explore potential biomarkers of infection or host response. Details for sample collection and processing are included in the lab manual.

Genotyping

Subjects' signed and dated informed consent specifically for genotyping assessments are obtained before conducting any sample collections. For part C only, blood sample for Fc Receptors for IgG (FcγR) and IgG1 allele genotyping is collected per the Schedule of Assessments in FIG. 29 to evaluate potential relationships with FluAB_MLNS mechanism of action or PK.

Product

FluAB_MLNS is provided as 300 mg lyophilized solid in an air-tight stoppered glass vial. Upon reconstitution to 150 mg/mL with USP water for injection, the drug product, as administered, contains 20 mM Histidine, 5.3% sucrose, and 0.02% PS80 at pH 6. FluAB_MLNS is injected intramuscularly. The unit dose is based on volume (0.8 mL to 4 mL per injection). No special procedures for safe handling of FluAB_MLNS are required. The FluAB_MLNS is stored in a secure, temperature-controlled environment.

Placebo is a sterile, preservative-free normal saline 0.9% solution for IM injection.

Statistical Methods

Part A and Part B

Statistical analyses are primarily descriptive. All study data is presented in data listings. Summary tables will present results by cohort for each FluAB_MLNS dose and placebo, as appropriate. Descriptive statistics are presented for continuous variables, and frequencies and percentages are presented for categorical and ordinal variables. Percentages are based on the number of non-missing values in a dose group. The impact of ADA on PK, and association with AEs and SAEs may be assessed.

Part C

The primary analysis population for efficacy analysis is the Full Analysis Set (FAS), which includes all randomized subjects who receive any amount of study drug. The primary efficacy endpoint is the proportion of subjects with laboratory-confirmed (by RT-PCR) influenza A illness, defined as ≥1 respiratory and ≥1 systemic symptom. The primary analysis consists of superiority tests of FluAB_MLNS compared to placebo based on the reduction of protocol-defined influenza A illness rate. If two dose levels are evaluated, the following hypotheses is tested according to the sequential testing principle at the 2-sided 0.05 level. If a null hypothesis is not rejected, formal sequential testing is stopped, and only nominal significance is reported for the remaining hypotheses:
1. Superiority of FluAB_MLNS dose level 2 (high) compared to placebo based on the protocol defined influenza A illness rate
2. Superiority of FluAB_MLNS dose level 1 (low) compared to placebo based on the protocol defined influenza A illness rate

If two dose levels are evaluated in Part C, a sample size of 460 subjects in each FluAB_MLNS group a 230 subjects in each of the matching placebo group provides approximately 80% power to detect a 70% reduction in the protocol defined illness rate (from 4.5% to 1.35%) between the placebo group and the FluAB_MLNS group respectively, using a 2-sided 0.05-level test. A total of approximately 1380 subjects are enrolled.

If only one dose level is evaluated, a total of approximately 920 subjects are enrolled. A sample size of 460 subjects in FluAB_MLNS group and 460 subjects in the placebo group provides approximately 80% power to detect a 70% reduction in the protocol defined illness rate (from 4.5% to 1.35%) between the placebo group and the VIR-2482 group respectively, using a 2-sided 0.05-level test.

If the interim analysis triggers the enrollment of additional subjects, approximately 1380 additional subjects are enrolled, if two doses levels are evaluated. The total study sample size is approximately 2760 subjects. If only one dose level is evaluated, approximately 920 additional subjects are enrolled. The total study sample size will be approximately 1840. A sample size of 920 subjects in FluAB_MLNS group and 920 subjects in the placebo group provide approximately 80% power to detect a 70% reduction in the protocol defined illness rate (from 2.25% to 0.675%) between the placebo group and the FluAB_MLNS group respectively, using a 2-sided 0.05-level test.

Endpoints

Part A

Primary endpoint is:
The safety and tolerability of FluAB_MLNS as measured by the incidence of treatment-emergent adverse events (TEAEs) and clinical assessments

The secondary endpoints are:
Single-dose FluAB_MLNS serum PK parameters (e.g., Cₘₐₓ, Cₗₐₛₜ, Tₘₐₓ, Tₗₐₛₜ, AUC_{inf}, AUCₗₐₛₜ, % AUC_{exp, t1/2}, λ_{z}, V_{z}/F, CL/F)
Incidence and titers (if applicable) of serum ADA to FluAB_MLNS

The exploratory endpoints may include:
Single-dose FluAB_MLNS nasopharyngeal secretion PK parameters (e.g., C, Cₗₐₛₜ, Tₘₐₓ, Tₗₐₛₜ, AUC_{inf}, AUCₗₐₛₜ, % AUCₑₓₚ, t_{1/2}, λ_{z}, V_{z}/F, CL/F)

Part B

The primary endpoint is:
Incidence and titers (if applicable) of ADA to FluAB_MLNS

The secondary endpoints are:
The safety and tolerability of FluAB_MLNS as measured by the incidence of treatment-emergent adverse events (TEAEs) and clinical assessments
FluAB_MLNS serum PK parameters (e.g., Cₘₐₓ, Clast, Tₘₐₓ, Tₗₐₛₜ, AUC_{inf}, AUClast, % AUCₑₓₚ, t_{1/2}, λ_{z}, V_{z}/F, CL/F)

Part C

The primary endpoints are:
The safety and tolerability of FluAB_MLNS as measured by the incidence of treatment-emergent adverse events (TEAEs) and clinical assessments.
Efficacy: Proportion of subjects with laboratory-confirmed (by RT-PCR) influenza A illness, defined as ≥1 respiratory and ≥1 systemic symptom

The secondary endpoints are:
Proportion of subjects with culture-confirmed influenza A illness
Severity and duration of subject-reported signs and symptoms of ILI due to influenza A
Quantification of the viral load present in nasopharyngeal secretions at the time of initial symptomatic presentation by RT-qPCR and viral culture
PK of FluAB_MLNS in serum
Incidence and titers (if applicable) of ADA to FluAB_MLNS

The exploratory endpoints may include:
Effect of FluAB_MLNS on potential biomarkers for host response after ILI presentation
Emergence of viral resistance to FluAB_MLNS in subjects presenting with influenza A illness
FcR polymorphisms as determined by genotyping and potential relationships with FluAB_MLNS mechanisms of action and/or PK
IgG1 allotypes as determined by genotyping
Rate of medically attended healthcare visits during the ILI monitoring period
Measure of Work Productivity and Activity Impairment (WPAI) due to influenza A illness

### LIST OF ABBREVIATIONS AND DEFINITIONS OF TERMS IN THIS EXAMPLE

ADA anti-drug antibody

ADE antibody-dependent enhancement

AE adverse event

ALT alanine aminotransferase

ALP alkaline phosphatase

AST aspartate aminotransferase

AUC area under the curve

BLQ below the limit of quantitation

BMI body mass index

BUN blood urea nitrogen

CLcr creatinine clearance

CMC Chemistry, Manufacturing, and Controls

CTCAE Common Terminology Criteria for Adverse Events

DMC Data Monitoring Committee

EC Ethics Committee

ECG electrocardiogram

eCRF electronic case report form

EOS End of Study

ET Early Termination

FcR Fc receptors for IgG

FDA Food and Drug Administration

GCP Good Clinical Practice

GGT gamma glutamyl transferase

GLP Good Laboratory Practice

GMP Good Manufacturing Practices

HA hemagglutinin

HED human equivalent dose

Hgb hemoglobin

ICF informed consent form

ICH International Conference on Harmonisation

IgG immunoglobulin G

ILI influenza-like illness

IM intramuscular

IND Investigational New Drug

INR International Normalized Ratio

IP investigational product

IRB Institutional Review Board

IV intravenous

IWRS interactive web response system

LDH lactate dehydrogenase

LLN lower limit of normal

LLOQ lower limit of quantitation

LLT Lower-Level Term

mAb monoclonal antibody

MedDRA Medical Dictionary for Regulatory Activities

NOAEL no observed adverse effect level

OTC over-the-counter

PK pharmacokinetics

POC Proof of concept

RBC red blood cell

SAD single ascending dose

SAE serious adverse event

SD standard deviation

SOC System Organ Class

SRC Safety Review Committee

SUSAR suspected unexpected serious adverse reaction

ULN upper limit of normal

WBC white blood cell

WHO World Health Organization

WOCBP women of child-bearing potential

WPAI Work Productivity and Activity Impairment

### Example 8

### Binding to Human FcRn at Different pHs

FluAB_wt and FluAB_MLNS were compared side by side for their ability to bind to neonatal Fc receptor (FcRn) using biolayer interferometry (BLI). To this end, binding of FluAB_wt and FluAB_MLNS to human FcRn was measured on an Octet RED96 instrument (biolayer interferometry, BLI, ForteBio). Biosensors coated with anti-human Fab-CHI were pre-hydrated in kinetic buffer for 10 min at RT. Then, human mAb (FluAB_wt or FluAB_MLNS) was loaded at 1 μg/ml in kinetics buffer at pH 7.4 for 30 minutes onto the Biosensors. The baseline was measured in kinetics buffer (Sterile filtered 0.01% endotoxin-free bovine serum albumin, 0.002% Tween-20 (Polysorbate 20), 0.005% NaN3 in PBS) at pH=7.4 or pH=6.0 for 4 minutes. Human mAb-loaded sensors were then exposed for 7 minutes to a solution of human FcRn at 1 μg/ml in kinetics buffer at pH=7.4 or pH=6.0 to measure association of FcRn-mAb in different milieus (on rate). Dissociation was then measured in kinetics buffer at the same pH for additional 5 minutes (off rate). All steps were performed while stirring at 1000 rpm at 30° C. Association and dissociation profiles were measured in real time as change in the interference patterns.

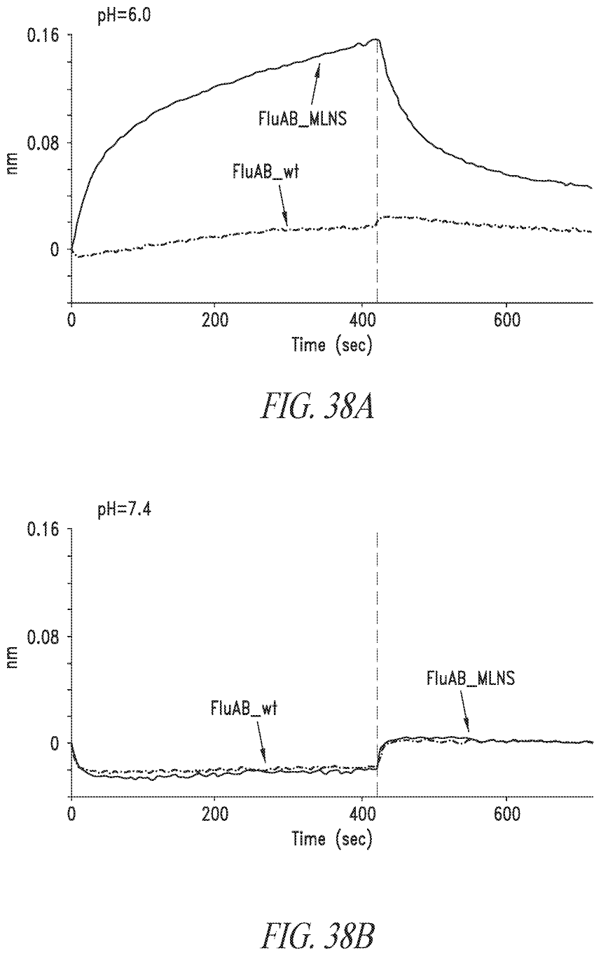

As shown in FIGS. 38A and 38B, FluAB_MLNS bound human FcRn with higher affinity compared to FluAB_wt at acidic pH (pH 6.0), while neither FluAB_MLNS nor FluAB_wt binds FcRn at neutral pH (pH 7.4).

### Example 9

### Characterization of Polymorphisms Identified in the Antibody's Extended Epitope

Historical polymorphisms in the extended epitope were evaluated for their impact on neutralization activity of FluAB_MLNS using viruses generated by reverse genetics with H1 HA or H3 HA on a A/Puerto Rico/8/34 (PR8) background.

Single nucleotide polymorphisms were introduced into PR8 H1 HA or A/Aichi/2/68 (Aichi) h-IA pHW2000 plasmids using site-directed mutagenesis. Recombinant influenza A virus were rescued with associated H1 or H3 HA on a PR8 backbone using standard methods (e.g., as described in Erich Hoffmann, Gabriele Neumann, Yoshihiro Kawaoka, Gerd Hobom, Robert G. Webster, 2000, A DNA transfection system for generation of influenza A virus from eight plasmids. Proceedings of the National Academy of Sciences May 2000, 97 (11): 61 08-6113; doi: -10.1073/pnas.t 00-133697).

Neutralization activity was evaluated in MDCK cells using standard methods. For example, neutralization activity may be evaluated in MDCK cells, e.g. in 96 well plates. To this end, MCDK cells may be seeded at 30,000 cells/well 24 hours prior to infection. Antibody FluAB_MLNS maybe incubated with virus for 1 hour at 37° C. prior to addition to MDCK cells.

To this end, 1:2.5 9-point serial dilutions of FluAB_MLNS may be created in infection media and each dilution may be tested in triplicate (e.g., 50 μg/mL−0.03 μg/mL final concentration) and may be incubated with 120 TCID₅₀ of virus for 1 hour at 37° C. MDCK cells may be washed twice with PBS, 100 μl/well of virus:antibody solution may be added, and cells may be incubated for 4 hours at 37° C. After 4 hours, an additional 100 μL/well of infection media may be added to cells. After 72 hours of incubation at 37° C., viral RNA may be extracted and measured by qRT-PCR, e.g. using WHO primers (World Health Organization. CDC protocol of real-time RT-PCR for influenza A H1N1. Apr. 28, 2009). The IC₅₀ is expressed as the antibody concentration in μg/mL that reduces 50% of virus replication and may be calculated using a non-linear 4-parameter logistic fit curve of data normalized to control wells (no virus and virus alone).

The neutralization activity of FluAB_MLNS to HI and H3 HA polymorphisms in the extended epitope is shown in Table 4 below.

TABLE 4

| | | FluAB_MLNS | |
|---|---|---|---|
| | Amino Acid | Geomean | Fold change |
| | Changes | Neutralization | relative |
| Virus | in HA | IC₅₀ (μg/mL) | to WT virus |
| PR8: Aichi HA wt | wild type | 5.6 | NA |
| PR8: Aichi HA P11S | P11S | 9.5 | 1.7 |
| PR8: Aichi HA D46N | D46N | 3.3 | 0.6 |
| PR8: Aichi HA N49T | N49T | 5.0 | 0.9 |
| PR8 wt | wild type | 4.7 | NA |
| PR8 HA N146D | N146D | 5.5 | 1.2 |

Aichi = A/Aichi/2/68;
Geomean = geometric mean;
HA = hemagglutinin;
NA = not applicable;
PR8 = A/Puerto Rico/8/34 H1N1;
wt = wild type

For viruses encoding H3 HA, FluAB_MLNS neutralized viruses with mutations HA1 P11S, HA2 D46N, or HA2 N49T with IC₅₀ values similar to wild type virus (<2-fold change in IC₅₀ relative to wild type virus). For viruses encoding H1 HA, FluAB_MLNS neutralized viruses encoding HA2 N146D with IC₅₀ values similar to wild type virus (<2-fold change in IC₅₀ relative to wild type virus). Additionally, the PR8 wild type strain used encoded the HA2 polymorphism L38Q and D46N and was neutralized with an IC₅₀ value of 4.7 ng/mL by FluAB_MLNS. Overall, all polymorphisms evaluated resulted in IC₅₀ fold changes of <2 relative to the wild type virus for FluAB_MLNS. In summary, FluAB_MLNS effectively neutralized all evaluated historical polymorphisms in the extended epitope (H3 HA: HA1 P11S, HA2 D46N, or HA2 N49T; H1 HA: N146D).

### Example 10

### Anti-Drug Antibody Response in Tg32 Mice

With regard to the M428L/N434S mutation, recently concerns were raised that said mutation increases immunogenicity of antibodies comprising this mutation (Brian C. Mackness, Julie A. Jaworski, Ekaterina Boudanova, Anna Park, Delphine Valente, Christine Mauriac, Olivier Pasquier, Thorsten Schmidt, Mostafa Kabiri, Abdullah Kandira, Katarina Radošević & Huawei Qiu (2019) Antibody Fc engineering for enhanced neonatal Fc receptor binding and prolonged circulation half-life, mAbs, 11:7, 1276-1288; Maeda A, Iwayanagi Y, Haraya K, et al. Identification of human IgG1 variant with enhanced FcRn binding and without increased binding to rheumatoid factor autoantibody. MAbs. 2017; 9(5):844-853).

To assess immunogenicity, in particular the anti-drug response (anti-drug antibodies; ADA), of antibody FluAB_MLNS in comparison to its parental antibody FluAB_wt, two separate groups (n=5) of TG32 mice (transgenic for the human FcRn) were injected i.v. with 5 mg/kg of either FluAB-MLNS or FluAB_wt monoclonal antibodies. To evaluate the circulating levels of the injected mAbs, blood samples were then obtained at different time points. Samples taken at day 14 and 21 post injection were used to evaluate, by specific ELISA, the anti-drug antibody (ADA) response against the injected human monoclonals.

Briefly, purified FluAB_wt and FluAB_MLNS monoclonal antibodies were coated on 96-well plates at 2 μg/ml. After blocking, the sera from treated animals obtained 14 and 21 days post injection, diluted 1: 180, were incubated 1.5 h at room temperature (RT). After washings, peroxidase-labeled goat anti-mouse IgG F(ab′)2 fragment (0.16 μg/ml) was added to plates and incubated 1.5 h at RT. ADA IgG (murine antibodies against the injected antibodies FluAB_wt and FluAB_MLNS) were then revealed with the appropriate substrate and read with a spectrophotometer. Data shown are the OD values (450 nm) obtained in each individual serum (n=5/group) collected 14 and 21 days after the antibody i.v. administration. Sera from naïve Tg32 mice (ctrl) were used as negative control.

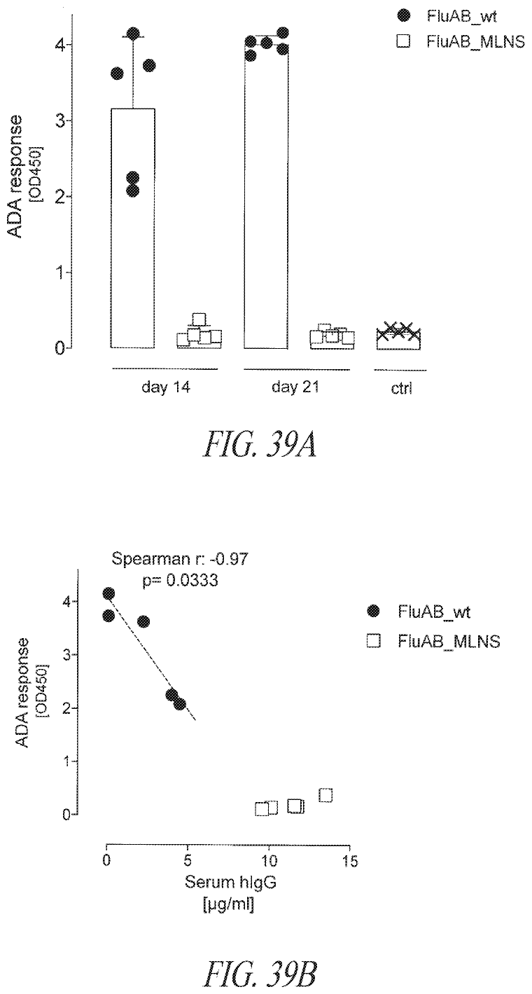

Results are shown in FIGS. 39A and 39B. Surprisingly, the signal of murine serum IgG reacting against the FluAB-MLNS antibody was very low and corresponding to the signal detected in control, non-injected animals, while the ADA response measured in the serum of mice injected with FluAB_wt was, instead, significantly high, both 14 and 21 day after i.v. injection (FIG. 39A). In addition, the levels of ADA measured at day 14 post injection significantly and inversely correlated with the levels of circulating FluAB_wt (serum FluAB_wt levels decreased due to murine antibodies against FluAB_wt), while the levels of circulating FluAB-MLNS measured at the same time were indeed much higher and homogeneous (FIG. 39B).

In summary, these data indicate that surprisingly the anti-drug response (anti-drug antibodies; ADA), and, thus, the immunogenicity of FluAB_MLNS was decreased compared to FluAB_wt.

### Example 11

### Anti-Drug Antibody Response and Immunogenicity After S.C. Administration

To further confirm this surprising finding in more immunogenic settings, separate groups of TG32 mice (n=5) were injected with either FluAB-MLNS or FluAB_wt (5 mg/kg) subcutaneously (s.c.), which is generally considered a more immunogenic route of administration. Three weeks after s.c. administration, the levels of anti-drug antibodies were measured in the serum by mouse anti-drug specific ELISA (as described above in Example 10) in the serum of mice injected s.c. with either FluAB_wt or FLuAB_MLNS. As negative control, a pool of 10 sera from naïve, untreated animals was used.

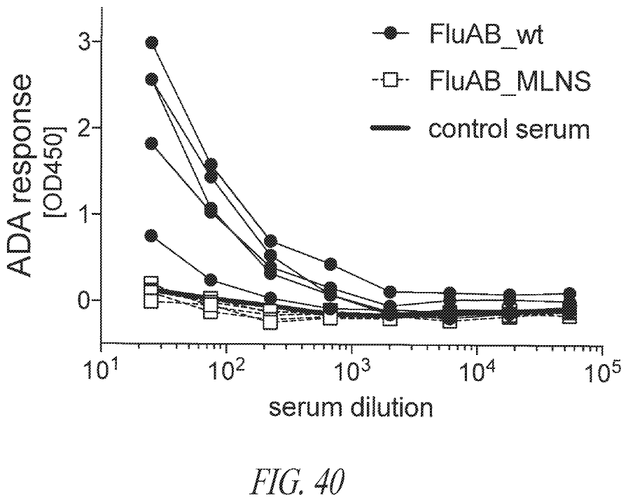

Results are shown in FIG. 40. Despite the more immunogenic settings, animals treated s.c. with FluAB-MLNS still did not mount a humoral immunogenic response, as confirmed by the serum titer of anti-hIgG antibodies that was overlapping to the one detected in the serum of non-injected control animals. Conversely, the ADA titer in animals treated with FluAB_wt was clearly positive and measurable in all treated animals. An inverse correlation between the circulating levels of injected antibody and anti hIgG endogenous response was detected in the sera of mice injected with FluAB_wt only (not shown).

These data surprisingly show that antibody FluAB_MLNS exhibits less immunogenicity as compared to its parental antibody FluAB_wt.

### Example 12

### In Vitro Neutralizing Activity

The ability of FluAB_WT to broadly neutralize influenza A viruses was evaluated in vitro in two separate studies using a microneutralization assay. A panel of 52 influenza A isolates collected from 1933-2014 was studied, representing 24 group 1 (subtypes H1, H2, H5, H6, and H9) and 28 group 2 viruses (subtypes H3 and H7). In one study, FluAB_WT neutralized all 37 viruses with a median half-maximal inhibitory concentration IC₅₀ of 0.78 μg/mL (ranging from 0.12 to 3.07 μg/mL). In the other study, FluAB_WT neutralized 15 additional virus strains isolated from 2010 to 2014 with a median IC₅₀ of 0.199 μg/mL (ranging from 0.067 to 2.69 μg/mL). The median IC₅₀ value for all group 1 and group 2 viruses in the two studies, excluding two 7:1 recombinant PR8 viruses, was 0.1 μg/mL for group 1 and 0.80 μg/mL for group 2 viruses (n=24 and n=26, respectively). In total, 17 H1N1 viruses were tested with a median IC₅₀ of 0.28 μg/mL and a IC₉₀ of 2.17 μg/mL. Accordingly, FluAB_WT can provide consistent neutralization activity despite naturally occurring antigenic drift. These data also support consistent neutralization activity for FluAB_MLNS.

### Table of Sequences and Seq Id Numbers (Sequence Listing)

| | | |
|---|---|---|
| SEQ ID NO | Sequence | Remarks |

FluAB_MLNS

| | | |
|---|---|---|
| SEQ ID NO: 1 | SYNAVWN | CDRH1 |
| SEQ ID NO: 2 | RTYYRSGWYNDYAESVKS | CDRH2 |
| SEQ ID NO: 3 | SGHITVFGVNVDAFDM | CDRH3 |
| SEQ ID NO: 4 | RTSQSLSSYTH | CDRL1 |
| SEQ ID NO: 5 | AASSRGS | CDRL2 |
| SEQ ID NO: 6 | QQSRT | CDRL3 |
| SEQ ID NO: 7 | QVQLQQSGPGLVKPSQTLSLTCAISGDSVSSY | VH |
| | NAVWNWIRQSPSRGLEWLGRTYYRSGWYND | |
| | YAESVKSRITINPDTSKNQFSLQLNSVTPEDTA | |
| | VYYCARSGHITVFGVNVDAFDMWGQGTMVT | |
| | VSS | |
| SEQ ID NO: 8 | DIQMTQSPSSLSASVGDRVTITCRTSQSLSSYT | VL |
| | HWYQQKPGKAPKLLIYAASSRGSGVPSRFSGS | |
| | GSGTDFTLTISSLQPEDFATYYCQQSRTFGQGT | |
| | KVEIK | |
| SEQ ID NO: 9 | QVQLQQSGPGLVKPSQTLSLTCAISGDSVSSY | Heavy chain |
| | NAVWNWIRQSPSRGLEWLGRTYYRSGWYND | |
| | YAESVKSRITINPDTSKNQFSLQLNSVTPEDTA | |
| | VYYCARSGHITVFGVNVDAFDMWGQGTMVT | |
| | VSSASTKGPSVFPLAPSSKSTSGGTAALGCLVK | |
| | DYFPEPVTVSWNSGALTSGVHTFPAVLQSSGL | |
| | YSLSSVVTVPSSSLGTQTYICNVNHKPSNTKV | |
| | DKRVEPKSCDKTHTCPPCPAPELLGGPSVFLFP | |
| | PKPKDTLMISRTPEVTCVVVDVSHEDPEVKFN | |
| | WYVDGVEVHNAKTKPREEQYNSTYRVVSVL | |
| | TVLHQDWLNGKEYKCKVSNKALPAPIEKTISK | |
| | AKGQPREPQVYTLPPSREEMTKNQVSLTCLVK | |
| | GFYPSDIAVEWESNGQPENNYKTTPPVLDSDG | |
| | SFFLYSKLTVDKSRWQQGNVFSCSVLHEALHS | |
| | HYTQKSLSLSPGK | |
| SEQ ID NO: 10 | DIQMTQSPSSLSASVGDRVTITCRTSQSLSSYT | Light chain |
| | HWYQQKPGKAPKLLIYAASSRGSGVPSRFSGS | |
| | GSGTDFTLTISSLQPEDFATYYCQQSRTFGQGT | |
| | KVEIKRTVAAPSVFIFPPSDEQLKSGTASVVCL | |
| | LNNFYPREAKVQWKVDNALQSGNSQESVTEQ | |
| | DSKDSTYSLSSTLTLSKADYEKHKVYACEVTH | |
| | QGLSSPVTKSFNRGEC | |

FluAB_wt

| | | |
|---|---|---|
| SEQ ID NO: 11 | QVQLQQSGPGLVKPSQTLSLTCAISGDSVSSY | Heavy chain |
| | NAVWNWIRQSPSRGLEWLGRTYYRSGWYND | |
| | YAESVKSRITINPDTSKNQFSLQLNSVTPEDTA | |
| | VYYCARSGHITVFGVNVDAFDMWGQGTMVT | |
| | VSSASTKGPSVFPLAPSSKSTSGGTAALGCLV | |
| | KDYFPEPVTVSWNSGALTSGVHTFPAVLQSSG | |
| | LYSLSSVVTVPSSSLGTQTYICNVNHKPSNTK | |
| | VDKRVEPKSCDKTHTCPPCPAPELLGGPSVFL | |
| | FPPKPKDTLMISRTPEVTCVVVDVSHEDPEVK | |
| | FNWYVDGVEVHNAKTKPREEQYNSTYRVVS | |
| | VLTVLHQDWLNGKEYKCKVSNKALPAPIEKT | |
| | ISKAKGQPREPQVYTLPPSREEMTKNQVSLTC | |
| | LVKGFYPSDIAVEWESNGQPENNYKTTPPVLD | |
| | SDGSFFLYSKLTVDKSRWQQGNVFSCSVMHE | |
| | ALHNHYTQKSLSLSPGK | |

All of the U.S. patents, U.S. patent application publications, U.S. patent applications, foreign patents, foreign patent applications and non-patent publications referred to in this specification or the attached Application Data Sheet are incorporated herein by reference, in their entirety to the extent not inconsistent with the present description. U.S. Provisional Application 62/893,747, filed Aug. 29, 2019, U.S. Provisional Application 62/993,519, filed Mar. 23, 2020, and U.S. Provisional Application 63/040,966 filed Jun. 18, 2020, are incorporated herein by reference, in their entirety. From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating or preventing an Influenza A infection in a subject, the method comprising administering to the subject a single dose of a pharmaceutical composition comprising an antibody, wherein the antibody comprises a light chain amino acid sequence according to SEQ ID NO:10 and a heavy chain amino acid sequence according to SEQ ID NO:9.

2. The method of claim 1, wherein the pharmaceutical composition comprises the antibody at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

3. The method of claim 1 or 2, wherein the single dose comprises 3, 4, 5, 6, or 7, preferably 5, mg of the antibody per kg of the subject's body weight.

4. The method of any one of claims 1-3, wherein the single dose comprises up to 60 mg, up to 300 mg, up to 1200 mg, up to 1800 mg, or up to 3000 mg of the antibody.

5. The method of any one of claims 1-4, wherein the single dose comprises up to 60 mg, up to 70 mg, up to 80 mg, up to 90 mg, up to 100 mg, up to 200 mg, up to 300 mg, up to 400 mg, up to 500 mg, up to 600 mg, up to 700 mg, up to 800 mg, up to 900 mg, up to 1000 mg, up to 1100 mg, up to 1200 mg, up to 1300 mg, up to 1400 mg, up to 1500 mg, up to 1600 mg, up to 1700 mg, up to 1800 mg, up to 2,000 mg, up to 2,500 mg, or up to 3000 mg, of the antibody.

6. The method of any one of claims 1-5, wherein the antibody is administered at a dose of 60 mg, 300 mg, 1200 mg, or 1800 mg.

7. The method of any one of claims 1-5, wherein the antibody is administered at a dose of 300 mg, 400 mg, 500 mg, 600 mg, 700 mg, 800 mg, 900 mg, 1100 mg, or 1200 mg.

8. The method of any one of claims 1-7, wherein:
(i) the single dose comprises 300 mg of the antibody, wherein the pharmaceutical composition comprises the antibody at 150 mg/mL, and the dose is administered by a single injection comprising 2 mL of the pharmaceutical composition;
(ii) the single dose comprises 1200 mg of the antibody, wherein the pharmaceutical composition comprises the antibody at 150 mg/mL, and the dose is administered by two injections each comprising 4 mL of the pharmaceutical composition;
(iii) the single dose comprises 1800 mg of the antibody, wherein the pharmaceutical composition comprises the antibody at 150 mg/mL, and the dose is administered by three injections each comprising 4 mL of the pharmaceutical composition; or
(iv) the single dose comprises 60 mg of the antibody, wherein the pharmaceutical composition comprises antibody at 150 mg/mL, and the dose is administered by one injection comprising 0.4 mL of the pharmaceutical composition.

9. The method of any one of claims 1-8, wherein the subject is human.

10. The method of any one of claims 1-9, wherein the method comprises intramuscular (IM) injection.

11. The method of any one of claims 1-10, wherein the pharmaceutical composition further comprises water (e.g., USP water for injection, or US sterile water for injection).

12. The method of any one of claims 1-11, wherein the pharmaceutical composition further comprises histidine, optionally at a concentration in a range from 10 mM to 40 mM, preferably 20 mM, in the pharmaceutical composition.

13. The method of any one of claims 1-12, wherein the pharmaceutical composition further comprises a sugar, such as a disaccharide, such as sucrose, optionally in a range from 3.0% to 9.0% (w/v), preferably in a range from 3.6% to 8.6%, more preferably in a range from 4% to 6%.

14. The method of any one of claims 1-13, wherein the pharmaceutical composition further comprises a surfactant or a triblock copolymer, optionally a polysorbate or poloxamer 188, preferably polysorbate 80 (PS80), optionally in a range from 0.01% to 0.05% (w/v), preferably 0.02% (w/v).

15. The method of any one of claims 1-14, wherein the pharmaceutical composition has a pH in a range from 5.5 to 6.5, or in a range from 5.8 to 6.2, or a pH of 5.5, 5.6, 5.7, 5.8, 5.9, 6.0, 6.1, 6.2, 6.3, 6.4, or 6.5, preferably of 6.0.

16. The method of any one of claims 1-15, wherein the single dose comprises from 0.8 mL to 4 mL per injection.

17. The method of claim 16, wherein the single dose comprises or consists of 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.1 mL, 3.2 mL, 3.3 mL, 3.4 mL, 3.5 mL, 3.6 mL, 3.7 mL, 3.8 mL, 3.9 mL, or 4.0 mL of the composition per injection.

18. The method of any one of claims 1-17, wherein at about 4 weeks, at about 12 weeks, and/or about 20 weeks following administering the pharmaceutical composition to the subject, the subject:
(i) has a reduced number and/or severity of a respiratory symptom selected from: cough, sore throat;, rhinorrhea; congestion; or any combination thereof, and/or
(ii) has a reduced number and/or severity of a systemic symptom selected from: fever [oral temperature >38° C. (100.4° F.)]; chills; myalgia; headache; malaise; fatigue; or any combination thereof,
as compared to a reference subject over a same time period who received a placebo or did not receive a therapy or vaccine for influenza A.

19. The method of any one of claims 1-18, wherein the subject is from 18 years to 65 years of age and has a body mass index in a range from 18 kg/m² to 32 kg/m² or in a range from 18 kg/m² to 35 kg/m².

20. The method of any one of claims 1-19, comprising administering the single dose comprising the pharmaceutical composition once to the subject during a six-month period.

21. The method of any one of claims 1-20, comprising administering a single dose comprising the pharmaceutical composition once to the subject during a twelve-month period.

22. The method of any one of claims 1-20, comprising administering a single dose comprising the pharmaceutical composition twice to the subject during a six-month period, such as once every three months.

23. The method of any one of claims 1-22, comprising administering the single dose comprising the pharmaceutical composition within 1-2 months (i.e., within 30 days to within 60 days) prior to the beginning of an influenza season, or within the first 1-2 months of the influenza season.

24. The method of any one of claims 1-23, wherein the antibody, or the pharmaceutical composition comprising the antibody, has an in vitro influenza inhibition of infection IC₉₀ of about 2.17 μg/mL.

25. The method of any one of claims 1-24, wherein:
(i) the administered pharmaceutical composition comprises 60 mg of the antibody, and the antibody is present in serum from the subject at a concentration from about 1 μg/mL to about 7 μg/mL for up to 120 days following administration;
(ii) the administered pharmaceutical composition comprises 300 mg of the antibody, and the antibody is present in serum from the subject at a concentration from about 8 μg/mL to about 20 μg/mL for up to 120 days following administration;
(iii) the administered pharmaceutical composition comprises 1200 mg of the antibody, and the antibody is present in serum from the subject at a concentration from about 50 to μg/mL to about 100 μg/mL for up to 120 days following administration;
(iv) the administered pharmaceutical composition comprises 1800 mg of the antibody, and the antibody is present in serum from the subject at a concentration from about 70 to μg/mL to about 110 μg/mL for up to 120 days following administration; and/or
(v) the antibody has an in vivo t_{1/2} in the subject of from 49 to 68 days.

26. The method of any one of claims 1-25, wherein the antibody of the pharmaceutical composition has an in vivo t_{1/2} in a human subject of from 49 to 68 days, such as 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, or 68 days.

27. The method of any one of claims 1-26, wherein the subject does not experience an adverse event (AE), according to the Common Terminology Criteria for Adverse Events (CTCAE), optionally for up to 140 days after the single dose of the pharmaceutical composition is administered.

28. The method of any one of claims 1-27, wherein the subject does not experience a moderate adverse event (AE), according to the Common Terminology Criteria for Adverse Events (CTCAE), optionally for up to 140 days, after the single dose of the pharmaceutical composition is administered.

29. The method of any one of claims 1-28, wherein the subject does not experience a serious adverse event (AE), according to the Common Terminology Criteria for Adverse Events (CTCAE), optionally for up to 140 days, after the single dose of the pharmaceutical composition is administered.

30. The method of any one of claims 1-29, wherein:
(i) the single dose comprises 300 mg of the antibody, wherein the pharmaceutical composition comprises the antibody at 150 mg/mL, and the single dose comprises a single injection comprising 2 mL of the pharmaceutical composition;
(ii) the single dose comprises 1200 mg of the antibody, wherein the pharmaceutical composition comprises the antibody at 150 mg/mL, and the single dose comprises two injections each comprising 4 mL of the pharmaceutical composition;
(iii) the single dose comprises 1800 mg of the antibody, wherein the pharmaceutical composition comprises the antibody at 150 mg/mL, and the single dose comprises three injections each comprising 4 mL of the pharmaceutical composition; or
(iv) the single dose comprises 60 mg of the antibody, wherein the pharmaceutical composition comprises antibody at 150 mg/mL, and the single dose comprises 0.4 mL of the pharmaceutical composition.

31. A pharmaceutical composition comprising an antibody that comprises a light chain amino acid sequence according to SEQ ID NO:10 and a heavy chain amino acid sequence according to SEQ ID NO:9, wherein the antibody is present in the composition at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

32. The pharmaceutical composition of claim 31, wherein the pharmaceutical composition further comprises water (e.g., USP water for injection, or US sterile water for injection).

33. The pharmaceutical composition of claim 31 or 32, further the pharmaceutical composition further comprises histidine, optionally at a concentration in a range from 10 mM to 40 mM, preferably 20 mM, in the composition.

34. The pharmaceutical composition of any one of claims 31-33, the pharmaceutical composition further comprises a sugar, such as a disaccharide, such as sucrose, optionally in a range from 3.0% to 9.0% (w/v), preferably from 3.6% to 8.6%, more preferably in a range from 4% to 6%.

35. The pharmaceutical composition of any one of claims 31-34, the pharmaceutical composition further comprises a surfactant or a triblock copolymer, optionally a polysorbate or poloxamer 188, preferably polysorbate 80 (PS80), optionally in a range from 0.01% to 0.05% (w/v), preferably 0.02%.

36. The pharmaceutical composition of any one of claims 31-35, wherein the composition has a pH in a range from 5.5 to 6.5, or in a range from 5.8 to 6.2, or a pH of 5.5, 5.6, 5.7, 5.8, 5.9, 6.0, 6.1, 6.2, 6.3, 6.4, or 6.5, preferably of 6.0.

37. A, preferably glass, vial comprising the pharmaceutical composition of any one of claims 31-36.

38. A syringe comprising the pharmaceutical composition of any one of claims 31-36.


SEQUENCE LISTING (11 sequences)

SEQ ID NO: 1 - protein, 7 aa, Artificial Sequence
  Synthetic seqeunce CDRH1
SYNAVWN

SEQ ID NO: 2 - protein, 18 aa, Artificial Sequence
  Synthetic seqeunce CDRH2
RTYYRSGWYNDYAESVKS

SEQ ID NO: 3 - protein, 16 aa, Artificial Sequence
  Synthetic seqeunce CDRH3
SGHITVFGVNVDAFDM

SEQ ID NO: 4 - protein, 11 aa, Artificial Sequence
  Synthetic seqeunce CDRL1
RTSQSLSSYTH

SEQ ID NO: 5 - protein, 7 aa, Artificial Sequence
  Synthetic seqeunce CDRL2
AASSRGS

SEQ ID NO: 6 - protein, 5 aa, Artificial Sequence
  Synthetic seqeunce CDRL3
QQSRT

SEQ ID NO: 7 - protein, 128 aa, Artificial Sequence
  Synthetic seqeunce VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 8 - protein, 103 aa, Artificial Sequence
  Synthetic seqeunce VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 9 - protein, 458 aa, Artificial Sequence
  Synthetic seqeunce Heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 10 - protein, 210 aa, Artificial Sequence
  Synthetic seqeunce Light chain
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 458 aa, Artificial Sequence
  Synthetic seqeunce Heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK